(19) United States
(12) Patent Application Publication
(10) Pub. No.: US 2020/0152294 A1
(43) Pub. Date: May 14, 2020

(54) METHOD FOR CALIBRATING A DATA SET OF A TARGET ANALYTE USING AN ANALYTE-INSUSCEPTIBLE SIGNAL VALUE

(71) Applicant: SEEGENE, INC., Seoul (KR)

(72) Inventors: Jong Yoon CHUN, Seoul (KR); Young Jo LEE, Seoul (KR); Han Bit LEE, Seoul (KR)

(21) Appl. No.: 16/613,597

(22) PCT Filed: May 19, 2017
(86) PCT No.: PCT/KR2017/005230

§ 371 Date: Nov. 14, 2019

(51) Int. Cl. G16B 35/00; G16B 40/00; G16B 45/00

(52) U.S. Cl. CPC G16B 35/00; G16B 45/00; G16B 40/00

(57) ABSTRACT

The present invention relates to a method for calibrating a data set of a target analyte in a sample using an analyte-in-susceptible signal value, wherein the analyte-insusceptible signal value is provided by a background-representing signal value of the data set or by a total signal change value of a standard data set. The present method is very convenient and effective in removing the inter- and intra-instrument signal variations of data sets. Furthermore, since the present method can be configured in software, the instant method is capable of being applied universally to various analytical instruments (e.g., a real-time PCR instrument) regardless of manufacturer. Accordingly, the method by the present invention would be very useful in diagnostic data analysis.

### FIELD OF THE INVENTION

The present invention relates to a method for calibrating a data set of a target analyte in a sample using an analyte-insusceptible signal value.

### BACKGROUND OF THE INVENTION

A polymerase chain reaction (hereinafter referred to as “PCR”) which is most widely used for the nucleic acid amplification includes repeated cycles of denaturation of double-stranded DNA, followed by oligonucleotide primer annealing to the DNA template, and primer extention by a DNA polymerase (Mullis et al., U.S. Pat. Nos. 4,683,195, 4,683,202 and 4,800,159; Saiki et al., (1985) Science 230, 1350-1354).

A real-time polymerase chain reaction is one of PCR-based technologies for detecting a target nucleic acid molecule in a sample in a real-time manner. For detecting a specific target analyte, the real-time PCR uses a signal-generating means for generating a fluorescence signal being detectable in a proportional manner with the amount of the target molecule. The generation of fluorescence signals may be accomplished by using either intercalators generating signals when intercalated between double-stranded DNA or oligonucleotides carrying fluorescent reporter and quencher molecules. The fluorescence signals whose intensities are proportional with the amount of the target molecule are detected at each amplification cycle and plotted against amplification cycles, thereby obtaining an amplification curve or amplification profile curve.

A sample analysis using fluorescence signals is performed as follow. When a luminant is supplied with energy through a light source such as LED, electron of the luminant is excited to a higher quantum state, then the luminant emits a light of specific wavelength by relaxation of the orbital electron to its ground state. Analytical instrument converts the light of specific wavelength to an electric signal using photodiode or CCD and provides information needed for sample analysis. Although the same amount of a luminant in a sample is analyzed, each analytical instrument provides different signal values because of the uneven illuminations of the light source (e.g., LED) and the performance variations of the light-electricity conversion device in the respective instruments. Such a signal difference between instruments is called as an inter-instrument variation. In addition to the inter-instrument variation, the analysis results of a plurality of reactions performed for the same kind and the same amount of the target analyte by a single identical analytical instrument may have variations in signal level because of the difference in reaction environments such as the position of reaction well where the reaction is performed on the instrument or delicate differences in composition or concentration of the reaction mixture. Such a signal difference among the reactions in a single instrument is known as an intra-instrument variation. Furthermore, an electrical noise signal is generated by an analytical instrument itself even when a blank (matrix without analyte) is analyzed and it may be identified as a normal signal. However, such an electrical noise signal also creates a signal variation and is referred to as an instrument blank signal. The instrument blank signal is generated in a manner that a specific amount of signal value is added to or subtracted from the original signal value for each cycle.

For the precise and reliable analysis, such problems have to be solved and several methods are used to solve the problems. As a most basic solution, a hardware adjustment method is used. For instance, when the analytical instrument is manufactured, the property of some parts of each analytical instrument such as intensity of LED light source is calibrated or adjusted such that the level of an inter-instrument variation for the same sample is reduced and maintained within a proper range.

However, these prior art may have some limitations or shortcomings. The hardware adjustment method shows limited accuracy in calibration and an additional calibration is needed to remove a variation occurred by deterioration of the analytical instrument.

Furthermore, the hardware adjustment method can reduce only the inter-instrument variation but cannot reduce the intra-instrument variation.

Accordingly, there are strong needs in the art to develop novel approaches for calibrating the data set and reducing the inter- and intra-instrument variations without direct adjusting of hardware.

Throughout this application, various patents and publications are referenced and citations are provided in parentheses. The disclosure of these patents and publications in their entirety are hereby incorporated by references into this application in order to more fully describe this invention and the state of the art to which this invention pertains.

### SUMMARY OF THE INVENTION

The present inventors have made intensive researches to develop novel approaches for accurately and conveniently reducing a signal variation in a data set of a signal-generating process for a target analyte. As a result, we have found that the data set can be calibrated by applying an analyte-insusceptible signal value to signal values of the data set to provide a calibrated data set with better accuracy and convenience. Furthermore, we have found two types of values which can be used as the analyte-insusceptible signal value. Accordingly, it is an object of this invention to provide a method for calibrating a data set of a target analyte in a sample.

It is another object of this invention to provide a computer readable storage medium containing instructions to configure a processor to perform a method for calibrating a data set of a target analyte in a sample.

It is still another object of this invention to provide a device for analyzing a method for calibrating a data set of a target analyte in a sample.

It is further object of this invention to provide a computer program to be stored on a computer readable storage medium to configure a processor to perform a method for calibrating a data set of a target analyte in a sample.

Other objects and advantages of the present invention will become apparent from the detailed description to follow taken in conjugation with the appended claims and drawings.

### BRIEF DESCRIPTION OF THE DRAWINGS

FIG. 1 represents a flow diagram illustrating an embodiment of the present method for calibrating a data set of a target analyte in a sample.

FIG. 2a represents amplification curves of three groups of raw data sets obtained respectively from three instruments without a hardware adjustment to show the inter-instrument and the intra-instrument variation of background signals.

FIG. 2b represents baseline subtracted amplification curves of three groups of raw data sets obtained respectively from three instruments without a hardware adjustment and analytical results of the inter- and the intra-instrument coefficient of variation of the data sets.

FIG. 3a represents amplification curves of three groups of raw data sets obtained respectively from three instruments with a hardware adjustment to show the inter-instrument and the intra-instrument variation of background signals.

FIG. 3b represents baseline subtracted amplification curves of three groups of raw data sets obtained respectively from three instruments with a hardware adjustment and analytical results of the inter- and the intra-instrument coefficient of variation of the data sets.

FIG. 4a represents baseline subtracted amplification curves of three standard data sets obtained respectively from three instruments without a hardware adjustment and total signal change values of each standard data set.

FIG. 4b represents amplification curves of calibrated data sets obtained by calibration of three groups of raw data sets by the Signal Variation-based Normalization method (SVN) of present invention using a total signal change value (TSC), wherein the raw data sets are obtained respectively from three instruments without a hardware adjustment.

FIG. 4c represents baseline subtracted amplification curves of calibrated data sets obtained by calibration of three groups of raw data sets by the SVN using a TSC, wherein the raw data sets are obtained respectively from three instruments without a hardware adjustment and analytical results of the inter- and the intra-instrument coefficient of variation of the calibrated data sets.

FIG. 5a represents baseline subtracted amplification curves of three standard data sets obtained respectively from three instruments with a hardware adjustment and total signal change values of each standard data set.

FIG. 5b represents amplification curves of calibrated data sets obtained by calibration of three groups of raw data sets by the SVN of present invention using a TSC, wherein the raw data sets are obtained respectively from three instruments with a hardware adjustment.

FIG. 5c represents baseline subtracted amplification curves of calibrated data sets obtained by calibration of three groups of raw data sets by the SVN using a TSC, wherein the raw data sets are obtained respectively from three instruments with a hardware adjustment and analytical results of the inter- and the intra-instrument coefficient of variation of the calibrated data sets.

FIG. 6 represents baseline subtracted amplification curves of calibrated data sets obtained by calibration of three groups of raw data sets by the SVN using the TSC, followed by further calibration using a R-TSC, wherein the raw data sets are obtained respectively from three instruments without a hardware adjustment and analytical results of the inter- and the intra-instrument coefficient of variation of the calibrated data sets.

FIG. 7 represents baseline subtracted amplification curves of calibrated data sets obtained by calibration of three groups of raw data sets by the SVN using the TSC, followed by further calibration using a R-TSC, wherein the raw data sets are obtained respectively from three instruments with a hardware adjustment and analytical results of the inter- and the intra-instrument coefficient of variation of the calibrated data sets.

FIG. 8a represents melting curves of three groups of raw melting data sets obtained respectively from three instruments without a hardware adjustment.

FIG. 8b represents melting peaks obtained by plotting the derivatives of the raw melting data sets obtained respectively from three instruments without a hardware adjustment and analytical results of the inter- and the intra-instrument coefficient of variation of maximum derivatives of the data sets.

FIG. 9a represents melting curves of three groups of raw melting data sets obtained respectively from three instruments with a hardware adjustment.

FIG. 9b represents melting peaks obtained by plotting the derivatives of the raw melting data sets obtained respectively from three instruments with a hardware adjustment and analytical results of the inter- and the intra-instrument coefficient of variation of maximum derivatives of the data sets.

FIG. 10a represents melting curves of three standard melting data sets obtained respectively from three instruments without a hardware adjustment and total signal change values of each standard data set.

FIG. 10b represents melting curves of calibrated melting data sets obtained by calibration of three groups of raw melting data sets by the Signal Variation-based Normalization method (SVN) of present invention using a total signal change value (TSC), wherein the raw melting data sets are obtained respectively from three instruments without a hardware adjustment.

FIG. 10c represents melting peaks of calibrated melting data sets obtained by plotting the derivatives of the calibrated melting data sets obtained by calibration of three groups of raw melting data sets by the SVN using a TSC, wherein the raw melting data sets are obtained respectively from three instruments without a hardware adjustment and analytical results of the inter- and the intra-instrument coefficient of variation of the calibrated malting data sets.

FIG. 11a represents melting curves of three standard melting data sets obtained respectively from three instruments with a hardware adjustment and total signal change values of each standard data set.

FIG. 11b represents melting curves of calibrated melting data sets obtained by calibration of three groups of raw melting data sets by the Signal Variation-based Normalization method (SVN) of present invention using a total signal change value (TSC), wherein the raw melting data sets are obtained respectively from three instruments with a hardware adjustment.

FIG. 11c represents melting peaks of calibrated melting data sets obtained by plotting the derivatives of the calibrated melting data sets obtained by calibration of three groups of raw melting data sets by the SVN using a TSC, wherein the raw melting data sets are obtained respectively from three instruments with a hardware adjustment and analytical results of the inter- and the intra-instrument coefficient of variation of the calibrated malting data sets.

FIG. 12 represents melting peaks of calibrated melting data sets obtained by plotting the derivatives of the calibrated melting data sets obtained by calibration of three groups of raw melting data sets by the SVN of the present invention using TSC, followed by further calibration using a R-TSC, wherein the raw melting data sets are obtained respectively from three instruments without a hardware adjustment and analytical results of the inter- and the intra-instrument coefficient of variation of the calibrated melting data sets.

FIG. 13 represents melting peaks of calibrated melting data sets obtained by plotting the derivatives of the calibrated melting data sets obtained by calibration of three groups of raw melting data sets by the SVN of the present invention using TSC, followed by further calibration using a R-TSC, wherein the raw melting data sets are obtained respectively from three instruments with a hardware adjustment and analytical results of the inter- and the intra-instrument coefficient of variation of the calibrated melting data sets.

FIG. 14a represents baseline subtracted amplification curves of three standard data sets obtained respectively from three instruments without a hardware adjustment and calibration coefficients of each standard data set provided by TSC and R-TSC.

FIG. 14b represents baseline subtracted amplification curves of calibrated data sets obtained by calibration of three groups of raw data sets by the Signal Variation-based Normalization method (SVN) using a calibration coefficient, wherein the raw data sets are obtained respectively from three instruments without a hardware adjustment and analytical results of the inter- and the intra-instrument coefficient of variation of the calibrated data sets.

FIG. 15a represents baseline subtracted amplification curves of three standard data sets obtained respectively from three instruments with a hardware adjustment and calibration coefficients of each standard data set provided by TSC and R-TSC.

FIG. 15b represents baseline subtracted amplification curves of calibrated data sets obtained by calibration of three groups of raw data sets by the Signal Variation-based Normalization method (SVN) using a calibration coefficient, wherein the raw data sets are obtained respectively from three instruments with a hardware adjustment and analytical results of the inter- and the intra-instrument coefficient of variation of the calibrated data sets.

FIG. 16a represents melting curves of three standard melting data sets obtained respectively from three instruments without a hardware adjustment and calibration coefficients of each standard data set provided by TSC and R-TSC.

FIG. 16b represents melting peaks of calibrated melting data sets obtained by plotting the derivatives of the calibrated melting data sets obtained by calibration of three groups of raw melting data sets by the SVN using a calibration coefficient, wherein the raw melting data sets are obtained respectively from three instruments without a hardware adjustment and analytical results of the inter- and the intra-instrument coefficient of variation of the calibrated malting data sets.

FIG. 17a represents melting curves of three standard melting data sets obtained respectively from three instruments with a hardware adjustment and calibration coefficients of each standard data set provided by TSC and R-TSC.

FIG. 17b represents melting peaks of calibrated melting data sets obtained by plotting the derivatives of the calibrated melting data sets obtained by calibration of three groups of raw melting data sets by the SVN using a calibration coefficient, wherein the raw melting data sets are obtained respectively from three instruments with a hardware adjustment and analytical results of the inter- and the intra-instrument coefficient of variation of the calibrated malting data sets.

FIG. 18a represents amplification curves of calibrated data sets obtained by calibration of three groups of raw data sets by the Reference Signal-based Normalization method (RSN) of present invention, wherein the raw data sets are obtained respectively from three instruments without a hardware adjustment.

FIG. 18b represents baseline subtracted amplification curves of calibrated data sets obtained by calibration of three groups of raw data sets by the Reference Signal-based Normalization method (RSN), wherein the raw data sets are obtained respectively from three instruments without a hardware adjustment and analytical results of the inter- and the intra-instrument coefficient of variation of the calibrated data sets.

FIG. 18c represents amplification curves of calibrated data sets obtained by calibration of three groups of raw data sets by the RSN, wherein the raw data sets are obtained respectively from three instruments with a hardware adjustment.

FIG. 18d represents baseline subtracted amplification curves of calibrated data sets obtained by calibration of three groups of raw data sets by the RSN, wherein the raw data sets are obtained respectively from three instruments with a hardware adjustment and analytical results of the inter- and the intra-instrument coefficient of variation of the calibrated data sets.

FIG. 19a represents amplification curve of calibrated data sets obtained by calibration of three groups of raw data sets by Instrument Blank signal Subtraction and Reference Signal-based Normalization method (IBS-RSN) using a single reference cycle, wherein the raw data sets are obtained respectively from three instruments without a hardware adjustment.

FIG. 19b represents baseline subtracted amplification curves of calibrated data sets obtained by calibration of three groups of raw data sets by IBS-RSN using a single reference cycle, wherein the raw data sets are obtained respectively from three instruments without a hardware adjustment and analytical results of the inter- and the intra-instrument coefficient of variation of the calibrated data sets.

FIG. 19c represents amplification curve of calibrated data sets obtained by calibration of three groups of raw data sets by IBS-RSN using three reference cycles, wherein the raw data sets are obtained respectively from three instruments without a hardware adjustment.

FIG. 19d represents baseline subtracted amplification curves of calibrated data sets obtained by calibration of three groups of raw data sets by IBS-RSN using three reference cycles, wherein the raw data sets are obtained respectively from three instruments without a hardware adjustment and analytical results of the inter- and the intra-instrument coefficient of variation of the calibrated data sets.

FIG. 20a represents amplification curve of calibrated data sets obtained by calibration of three groups of raw data sets by Instrument Blank signal Subtraction and Reference Signal-based Normalization method (IBS-RSN) using a single reference cycle, wherein the raw data sets are obtained respectively from three instruments with a hardware adjustment.

FIG. 20b represents baseline subtracted amplification curves of calibrated data sets obtained by calibration of three groups of raw data sets by IBS-RSN using a single reference cycle, wherein the raw data sets are obtained respectively from three instruments with a hardware adjustment and analytical results of the inter- and the intra-instrument coefficient of variation of the calibrated data sets.

FIG. 20c represents amplification curve of calibrated data sets obtained by calibration of three groups of raw data sets by IBS-RSN using three reference cycles, wherein the raw data sets are obtained respectively from three instruments with a hardware adjustment.

FIG. 20d represents baseline subtracted amplification curves of calibrated data sets obtained by calibration of three groups of raw data sets by IBS-RSN using three reference cycles, wherein the raw data sets are obtained respectively from three instruments with a hardware adjustment and analytical results of the inter- and the intra-instrument coefficient of variation of the calibrated data sets.

### DETAILED DESCRIPTION OF THIS INVENTION

### I. Method for Calibrating a Data Set of a Target Analyte Using Analyte-Insusceptible Signal Value

In one aspect of this invention, there is provided a method for calibrating a data set of a target analyte in a sample comprising:

(a) providing an analyte-insusceptible signal value for calibrating the data set; wherein the data set is obtained from a signal-generating process for the target analyte using a signal-generating means; wherein the data set comprises a plurality of data points comprising cycles and signal values of the signal-generating process;

wherein the analyte-insusceptible signal value is provided (i) by a background-representing signal value of the data set; wherein the background-representing signal value is provided by a signal value at a reference cycle of the data set and the reference cycle is selected within a background region of the data set where signal generation is insusceptible to the presence or absence of the target analyte in the sample; or (ii) by a total signal change value of a standard data set; wherein the standard data set is obtained by a signal-generating process for a standard material of the target analyte; and

(b) providing a calibrated data set by obtaining calibrated signal values by applying the analyte-insusceptible signal value to the signal values of the data set.

The present inventors have made intensive researches to develop novel approaches for the calibration of a data set which enables us to more efficiently and accurately reduce the inter- and intra-instrument signal variations in a data set representing the presence or absence of a target analyte (e.g., target nucleic acid molecules). As a result, we have found that a calibrated data set suitable for a sample analysis can be obtained by providing an analyte-insusceptible signal value and applying the analyte-insusceptible signal value to signal values of a plurality of data points of the data set.

The term used herein “calibration” or “adjustment” refers to a correction of a data set, particularly a correction of a signal value of a data set which becomes suitable for the aim of analysis.

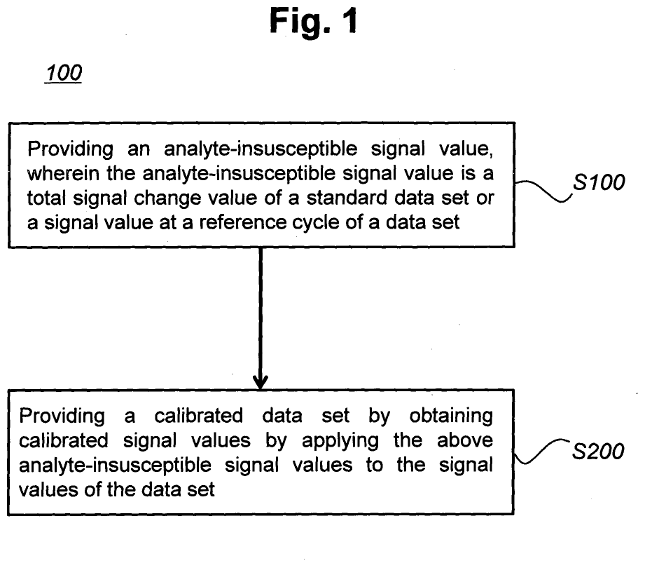

FIG. 1 represents a flow diagram illustrating an embodiment of the present method for calibrating a data set of a target analyte in a sample.

The present invention will be described in more detail as follows:

### Step (a): Providing an Analyte-Insusceptible Signal Value for Calibrating a Data Set (S110)

According to the present method, an analyte-insusceptible signal value for calibrating a data set is provided. The data set is obtained from a signal-generating process for a target analyte using a signal-generating means, and the data set comprises a plurality of data points comprising cycles and signal values of the signal-generating process.

The terms used herein a target analyte may include various materials (e.g., biological materials and non-biological materials such as chemicals). Particularly, the target analyte may include biological materials such as nucleic acid molecules (e.g., DNA and RNA), proteins, peptides, carbohydrates, lipids, amino acids, biological chemicals, hormones, antibodies, antigens, metabolites and cells. More particularly, the target analyte may include nucleic acid molecules. According to an embodiment, the target analyte may be a target nucleic acid molecule.

The term used herein “sample” may include biological samples (e.g., cell, tissue and fluid from a biological source) and non-biological samples (e.g., food, water and soil). The biological samples may include virus, bacteria, tissue, cell, blood (e.g., whole blood, plasma and serum), lymph, bone marrow aspirate, saliva, sputum, swab, aspiration, milk, urine, stool, vitreous humour, sperm, brain fluid, cerebrospinal fluid, joint fluid, fluid of thymus gland, bronchoalveolar lavage, ascites and amnion fluid. When a target analyte is a target nucleic acid molecule, the sample is subjected to a nucleic acid extraction process. When the extracted nucleic acid is RNA, reverse transcription process is performed additionally to synthesize cDNA from the extracted RNA (Joseph Sambrook, et al., Molecular Cloning, A Laboratory Manual, Cold Spring Harbor Laboratory Press, Cold Spring Harbor, N.Y. (2001)).

The term used herein “signal-generating process” refers to any process capable of generating signals in a dependent manner on a property of a target analyte in a sample, wherein the property may be, for instances, activity, amount or presence (or absence) of the target analyte, in particular the presence of (or the absence of) an analyte in a sample. According to an embodiment, the signal-generating process generates signals in a dependent manner on the presence of the target analyte in the sample.

Such signal-generating process may include biological and chemical processes. The biological processes may include genetic analysis processes such as PCR, real-time PCR, microarray and invader assay, immunoassay processes and bacteria growth analysis. According to an embodiment, the signal-generating process includes genetic analysis processes. The chemical processes may include a chemical analysis comprising production, change or decomposition of chemical materials. According to an embodiment, the signal-generating process may be a PCR or a real-time PCR.

The signal-generating process may be accompanied with a signal change. The term “signal” as used herein refers to a measurable output. The signal change may serve as an indicator indicating qualitatively or quantitatively the property, in particular the presence or to absence of a target analyte. Examples of useful indicators include fluorescence intensity, luminescence intensity, chemiluminescence intensity, bioluminescence intensity, phosphorescence intensity, charge transfer, voltage, current, power, energy, temperature, viscosity, light scatter, radioactive intensity, reflectivity, transmittance and absorbance. The most widely used indicator is fluorescence intensity. The signal change may include a signal decrease as well as a signal increase. According to an embodiment, the signal-generating process is a process of amplifying the signal values.

The term used herein “signal-generating means” refers to any material used in the generation of a signal indicating a property, more specifically the presence or absence of the target analyte which is intended to be analyzed.

A wide variety of the signal-generating means have been known to one of skill in the art. Examples of the signal-generating means may include oligonucleotides, labels and enzymes. The signal-generating means include both labels per se and oligonucleotides with labels. The labels may include a fluorescent label, a luminescent label, a chemiluminescent label, an electrochemical label and a metal label. The label per se like an intercalating dye may serve as signal-generating means. Alternatively, a single label or an interactive dual label containing a donor molecule and an acceptor molecule may be used as signal-generating means in the form of linkage to at least one oligonucleotide. The signal-generating means may comprise additional components for generating signals such as nucleolytic enzymes (e.g., 5′-nucleases and 3′-nucleases).

Where the present method is applied to determination of the presence or absence of a target nucleic acid molecule, the signal-generating process may be performed in accordance with a multitude of methods known to one of skill in the art. The methods include TaqMan™ probe method (U.S. Pat. No. 5,210,015), Molecular Beacon method (Tyagi et al., Nature Biotechnology, 14 (3):303 (1996)), Scorpion method (Whitcombe et al., Nature Biotechnology 17:804-807 (1999)), Sunrise or Amplifluor method (Nazarenko et al., Nucleic Acids Research, 25(12):2516-2521 (1997), and U.S. Pat. No. 6,117,635), Lux method (U.S. Pat. No. 7,537,886), CPT (Duck P, et al., Biotechniques, 9:142-148 (1990)), LNA method (U.S. Pat. No. 6,977,295), Plexor method (Sherrill C B, et al., Journal of the American Chemical Society, 126:4550-4556 (2004)), Hybeacons™ (D. J. French, et al., Molecular and Cellular Probes (2001) 13, 363-374 and U.S. Pat. No. 7,348,141), Dual-labeled, self-quenched probe (U.S. Pat. No. 5,876,930), Hybridization probe (Bernard P S, et al., Clin Chem 2000, 46, 147-148), PTOCE (PTO cleavage and extension) method (WO 2012/096523), PCE-SH (PTO Cleavage and Extension-Dependent Signaling Oligonucleotide Hybridization) method (WO 2013/115442) and PCE-NH (PTO Cleavage and Extension-Dependent Non-Hybridization) method (PCT/KR2013/012312) and CER method (WO 2011/037306).

The term used herein “amplification” or “amplification reaction” refers to a reaction for increasing or decreasing signals. According to an embodiment of this invention, the amplification reaction refers to an increase (or amplification) of a signal generated depending on the presence of the target analyte by using the signal-generating means. The amplification reaction is accompanied with or without an amplification of the target analyte (e.g., nucleic acid molecule). Therefore, according to an embodiment of this invention, the signal-generating process is performed with or without an amplification of the target nucleic acid molecule. More particularly, the amplification reaction of present invention refers to a signal amplification reaction performed with an amplification of the target analyte.

The data set obtained from an amplification reaction comprises an amplification cycle.

The term used herein “cycle” refers to a unit of changes of conditions or a unit of a repetition of the changes of conditions in a plurality of measurements accompanied with changes of conditions. For example, the changes of conditions or the repetition of the changes of conditions include changes or repetition of changes in temperature, reaction time, reaction number, concentration, pH and/or replication number of a measured subject (e.g., target nucleic acid molecule). Therefore, the cycle may include a condition (e.g., temperature or concentration) change cycle, a time or a process cycle, a unit operation cycle and a reproductive cycle. A cycle number represents the number of repetition of the cycle. In this document, the terms “cycle” and “cycle number” are used interchangeably. For example, when enzyme kinetics is investigated, the reaction rate of an enzyme is measured several times as the concentration of a substrate is increased regularly. In this reaction, the increase in the substrate concentration may correspond to the changes of the conditions and the increasing unit of the substrate concentration may be corresponding to a cycle. For another example, when an isothermal amplification of nucleic acid is performed, the signals of a single sample are measured multiple times with a regular interval of times under isothermal conditions. In this reaction, the reaction time may correspond to the changes of conditions and a unit of the reaction time may correspond to a cycle. According to another embodiment, as one of methods for detecting a target analyte through a nucleic acid amplification reaction, a plurality of fluorescence signals generated from the probes hybridized to the target analyte are measured with a regular change of the temperature in the reaction. In this reaction, the change of the temperature may correspond to the changes of conditions and the temperature may correspond to a cycle.

Particularly, when repeating a series of reactions or repeating a reaction with a time interval, the term “cycle” refers to a unit of the repetition. For example, in a polymerase chain reaction (PCR), a cycle refers to a reaction unit comprising denaturation of a target nucleic acid molecule, annealing (hybridization) between the target nucleic acid molecule and primers and primer extension. The increases in the repetition of reactions may correspond to the changes of conditions and a unit of the repetition may correspond to a cycle.

According to an embodiment, where the target nucleic acid molecule is present in a sample, values (e.g., intensities) of signals measured are increased or decreased upon increasing cycles of an amplification reaction. According to an embodiment, the amplification reaction to amplify signals indicative of the presence of the target nucleic acid molecule may be performed in such a manner that signals are amplified simultaneously with the amplification of the target nucleic acid molecule (e.g., real-time PCR). Alternatively, the amplification reaction may be performed in such a manner that signals are amplified with no amplification of the target nucleic acid molecule [e.g., CPT method (Duck P, et al., Biotechniques, 9:142-148 (1990)), Invader assay (U.S. Pat. Nos. 6,358,691 and 6,194,149)]. The target analyte may be amplified by various methods. For example, a multitude of methods have been known for amplification of a target nucleic acid molecule, including, but not limited to, PCR (polymerase chain reaction), LCR (ligase chain reaction, see U.S. Pat. Nos. 4,683,195 and 4,683,202; A Guide to Methods and Applications (Innis et al., eds, 1990); Wiedmann M, et al., “Ligase chain reaction (LCR)-overview and applications.” PCR Methods and Applications 1994 February; 3(4):S51-64), GLCR (gap filling LCR, see WO 90/01069, EP 439182 and WO 93/00447), Q-beta (Q-beta replicase amplification, see Cahill P, et al., Clin Chem., 37(9):1482-5 (1991), U.S. Pat. No. 5,556,751), SDA (strand displacement amplification, see G T Walker et al., Nucleic Acids Res. 20(7):1691-1696 (1992), EP 497272), NASBA (nucleic acid sequence-based amplification, see Compton, J. Nature 350(6313):91-2 (1991)), TMA (Transcription-Mediated Amplification, see Hofmann W P et al., J Clin Virol. 32(4):289-93 (2005); U.S. Pat. No. 5,888,779) or RCA (Rolling Circle Amplification, see Hutchison C. A. et al., Proc. Natl Acad. Sci. USA. 102:17332-17336 (2005)).

According to an embodiment, the label used for the signal-generating means may comprise a fluorescence, more particularly, a fluorescent single label or an interactive dual label comprising donor molecule and acceptor molecule (e.g., an interactive dual label containing a fluorescent reporter molecule and a quencher molecule).

According to an embodiment, the amplification reaction used in the present invention may amplify signals simultaneously with amplification of the target analyte, particularly the target nucleic acid molecule. According to an embodiment, the amplification reaction is performed in accordance with a PCR or a real-time PCR.

The data set obtained from a signal-generating process comprises a plurality of data points comprising cycles of the signal-generating process and signal values at the cycles. The term used herein “values of signals” or “signal values” means either values of signals actually measured at the cycles of the signal-generating process (e.g., actual value of fluorescence intensity processed by amplification reaction) or their modifications. The modifications may include mathematically processed values of measured signal values (e.g., intensities). Examples of mathematically processed values of measured signal values may include logarithmic values and derivatives of measured signal values. The derivatives of measured signal values may include multi-derivatives.

The term used herein “data point” means a coordinate value comprising a cycle and a value of a signal at the cycle. The term used herein “data” means any information comprised in data set. For example, each of cycles and signal values of an amplification reaction may be data. The data points obtained from a signal-generating process, particularly from an amplification reaction may be plotted with coordinate values in a rectangular coordinate system. In the rectangular coordinate system, the X-axis represents cycles of the amplification reaction and the Y-axis represents signal values measured at each cycles or modifications of the signal values.

The term used herein “data set” refers to a set of data points. The data set may include a raw data set which is a set of data points obtained directly from the signal-generating process (e.g., an amplification reaction) using a signal-generating means. Alternatively, the data set may be a modified data set which is obtained by a modification of the data set including a set of data points obtained directly from the signal-generating process. The data set may include an entire or a partial set of data points obtained from the signal-generating process or modified data points thereof.

The term used herein “analyte-insusceptible signal value” means a signal value which is not correlated with the presence/absence or the amount of a target analyte in a sample. The variations of the analyte-insusceptible signal values between a plurality of data sets are caused by difference in various factors of reactions not by the presence/absence or the amount of a target analyte in a sample. Interestingly, where the data sets obtained from reactions are calibrated by using the analyte-insusceptible signal value, the variations between the plurality of data sets caused by various factors other than a target analyte can be reduced, thereby giving more reliable calibrated data sets.

According to an embodiment, the analyte-insusceptible signal value may be a signal value detected in a background region of a data set obtained from a real-time PCR. The signal value detected in a background region has little correlation with the presence/to absence or the amount of a target analyte in a sample.

According to another embodiment, the analyte-insusceptible signal value may be a total signal change value which is calculated from a data set obtained from a separate material not from the sample to be analyzed.

An example of the separate material may be a standard material. The term “standard material” used herein means any material that generates the substantially same level of signal values whenever it is applied to signal-generating processes performed under the same reaction conditions. Particularly, the standard material may generate a signal value reflecting differences in reaction conditions under which signal-generating processes are performed. The standard material is applied to each signal-generating process of a plurality of data sets to calibrate variations between the plurality of data sets.

The term “total signal change value” used herein means an overall signal change (increase or decrease) amount of a data-set. A total signal-change value may be calculated from a standard data set that is obtained through a signal-generating process for a standard material. The signal-generating process for a standard material may be performed in an instrument or in a well identical to that in which a data set of a sample to be analyzed is obtained. An inter-instrument or inter-well variation of a data set can be effectively reduced by calibrating the data set by using a total signal change value of a standard data set.

The analyte-insusceptible signal value can be provided by (i) a data set obtained from a signal-generating process using a sample to be analyzed, or (ii) a data set obtained from a signal-generating process using a separate material. These two embodiments will be described in more detail in the following sections.

According to an embodiment, the analyte-insusceptible signal value is (i) the background-representing signal value of the data set or (ii) the total signal change value of the standard data set obtained by the signal-generating process for the standard data set.

According to an embodiment, the data set of the step (a) may be a modified data set. According to an embodiment, the data set may be a modified data set of a raw data set. The modification may be mathematical modification. According to an embodiment, the data set may be a mathematically processed data set of the raw data set. In particular, the data set may be a baseline subtracted data set for removing a background signal value from the raw data set. The baseline subtracted data set may be obtained by methods well known in the art (e.g., U.S. Pat. No. 8,560,247).

The term “raw data set” as used herein refers to a set of data points (including cycle numbers and signal values) obtained directly from an amplification reaction. The raw data set means a set of non-processed data points which are initially received from a device for performing a real-time PCR (e.g., thermocycler, PCR machine or DNA amplifier). In an embodiment of the present invention, the raw data set may include a raw data set understood conventionally by one skilled in the art. In an embodiment of the present invention, the raw data set may include a dataset prior to processing. In an embodiment of the present invention, the raw data set may include a dataset which is the basis for the mathematically processed data sets as described herein. In an embodiment of the present invention, the raw data set may include a data set not subtracted by a baseline (no baseline subtraction data set).

According to an embodiment, the data set of step (a) may be a data set of which a blank signal value is removed.

The term “1^{st} calibrated data set” may be used herein in order to refer to a modified data set in which a blank signal value is removed from a raw data set. The 1^{st} calibrated data set may be interpreted as a modified data set and distinguished from a finally calibrated data set or a 2^{nd} calibrated data set.

According to an embodiment, the blank signal value may be a signal value obtained by no use of the signal-generating means. Particularly, the blank signal value is a signal value detected from a reaction performed without signal-generating means such as labels per se, or labeled oligonucleotides which generate a signal by the presence of the target analyte. Because such blank signal value is measured in the absence of the signal-generating means, a signal variation due to an instrument-to-instrument difference in ratios of signals generated per unit concentration of target analytes is not applied to the blank signal value.

The blank signal may be determined and applied in various approaches. For example, the separate blank signal values may be determined for applying to their corresponding instruments. A single blank signal may be applied to data sets obtained by a single instrument and different blank signals each may be applied to the data sets obtained each of their corresponding instruments. Alternatively, different blank signals each may be determined for applying to each of wells within a single instrument. Each well within a single instrument may have its own blank signal and different blank signals each may be applied to data sets obtained by each of their corresponding wells within a single instrument.

The data set which is removed of a blank signal may be the data set in which a blank signal is removed in whole or in part. The term of “removal” means subtracting or adding a value of signal from/to a data set. Particularly, the term “removal” refers to subtraction of a value of signal from a data set. When a blank signal has a negative value, it may be removed by adding a value of signal.

According to an embodiment, the blank signal value may be obtained by no use of the signal-generating means. Particularly, the blank signal value may be measured using an empty well, an empty tube, a tube containing water or a tube containing a real-time PCR reaction mixture without signal-generating means such as fluorescence molecule conjugated oligonucleotide. A measurement of a blank signal value may be performed together with a signal-generating process or may be performed separately from a signal-generating process. According to an embodiment, a blank signal value may be removed in whole in such a manner that measurement of the blank signal is performed together with a signal-generating process and the measured blank signal value is subtracted from signal values of a data set obtained by the signal-generating process.

Alternatively, a blank signal may be removed in part in such a manner that a certain value of a signal is subtracted from signal values of a data set obtained by a signal-generating process. The certain value of a signal may be any value so long as a signal corresponding to a blank signal in a data set is reduced by the subtraction of the certain value of signal. For instance, the certain value of a signal may be determined based on a plurality of blank signals measured from one instrument or a plurality of instruments. When it is troublesome to measure a blank signal for each target analyte analysis experiment, a blank signal may be removed from data sets in such a manner that a certain value of signal corresponding to a portion of the blank signal value is determined based on a plurality of blank signal values measured from one instrument or from a plurality of instruments and then the determined certain value of signal is subtracted from each of data sets.

Alternatively, the certain value of signal may be determined in such a range that a signal variation of a data set is reduced when the certain value of signal is subtracted from the data set and the signal values of the subtracted data set are calibrated with ratio according to the present method. As such, the data set reduced of a blank signal may be provided by subtracting the certain value of signal which is a portion of the blank signal without measurement of a blank signal value for each reaction.

The method of the present invention may be a method for calibrating a single data set of a target analyte in a sample. Alternatively, the method for present invention may be a method for calibrating a plurality of data sets. According to an embodiment, the signal-generating process is a plurality of signal-generating processes for the same-typed target analyte performed in different reaction vessels and the data set is a plurality of data sets obtained from the plurality of signal-generating processes.

According to an embodiment, the signal-generating process may be a plurality of signal-generating processes for the detection of the same type of target analytes, the data set may be a plurality of data sets.

The plurality of signal-generating processes may be a plurality of signal-generating processes for the detection of the same type of target analytes (i.e, the same-typed target analytes). The same type of target analytes may be a plurality of target analytes isolated from the same kind of samples. Alternatively, the same type of target analytes may be a plurality of target analytes which is isolated from the different kinds of samples but detected by the same signal-generating means (e.g., the same probes or same primers).

According to an embodiment, the plurality of signal-generating processes may be a plurality of signal-generating processes for the same-typed target analyte performed in different reaction environments. Signal-generating processes in different reaction environments comprise various embodiments. Particularly, the signal-generating processes in different reaction environments may be a signal-generating processes performed on different instruments, performed in different wells or reaction tubes, performed for different samples, performed with target analytes of different concentrations, performed with different primers or probes, performed with different signal-generating dyes or performed by different signal-generating means. According to an embodiment, the plurality of the signal-generating-processes is performed on different instruments from each other.

The plurality of data sets may be obtained by using a plurality of samples. Samples in the plurality of samples may be different from each other, particularly, at least two samples in the plurality of samples may be different from each other. Alternatively, Samples in the plurality of samples may be identical to each other.

The inter-instrument variation may be a signal variation between the separate data sets which are obtained by the signal-generating processes for the identical target analyte performed on the respective different instruments. Alternatively, the inter-instrument variation may be a signal variation between the separate data sets which are obtained by independent operations of the signal-generating processes for the identical target analyte on the identical instrument. For example, the independent operations of the signal-generating processes for the identical target analyte may be performed on the identical instrument with an operation time interval. In this case, the independent operation of an instrument may be considered as an instrument.

According to an embodiment, the signal-generating process may comprise a plurality of signal-generating processes for the same-typed target analyte performed in different reaction vessels, and the data set may comprise a plurality of data sets obtained from the plurality of signal-generating processes. The plurality of signal-generating processes may be performed in different reaction vessels. The term used herein “reaction vessel” refers to a vessel or a portion of a device at which a reaction is processed by mixing a sample and signal-generating means (e.g., primers or probes). The expression used herein “the plurality of signal-generating processes may be performed in different reaction vessels” means that a signal-generating process is performed using a signal-generating means and a sample that are separated from another signal-generating means and sample for another signal-generating process. For example, the signal-generating processes performed in a plurality of tubes or in a plurality of wells of a plate may correspond to the plurality of signal-generating processes. The signal-generating processes which are performed in the same reaction vessel but in different times also may correspond-to-the-plurality of signal-generating processes.

According to an embodiment, the method of the present invention may further comprises the step of removing abnormal signals (e.g., spike signal or jump error) from a data set obtained by a signal-generating process before providing an analyte-insusceptible signal value used for calibrating the data set.

### Step (b): Providing a Calibrated Data Set by Obtaining Calibrated Signal Values by Applying the Analyte-Insusceptible Signal Value (S120)

A calibrated data set may be provided by obtaining calibrated signal values by applying the analyte-insusceptible signal value to the signal values of the data set. Particularly, a calibrated data set may be provided by obtaining calibrated signal values by applying the analyte-insusceptible signal value to the signal values of the plurality of data points of the data set.

The analyte-insusceptible signal value may be applied to the signal values of the data set by various approaches.

According to an embodiment, a calibrated data set in the step (b) may be provided by obtaining calibrated signal values by dividing the signal values of the data set by the analyte-insusceptible signal value.

According to an embodiment, the calibrated signal value is obtained by using the following mathematical equation 1:

Calibrated signal value=signal value/analyte-insusceptible signal value Equation 1

The signal value in Equation 1 is uncalibrated signal value. The uncalibrated signal value refers to a signal value of a data set before the data set is calibrated by the analyte-insusceptible signal value. Therefore, the uncalibrated signal value may be a measured signal value or a processed signal value of the measured signal value. The process may be a process performed independently from a calibration process using the analyte-insusceptible signal value. For example, the signal value processing may be performed by adding or subtracting a certain value to or from the signal value. Particularly, the process may be removing a blank signal in whole or in part from the measured signal value.

The calibrated signal value refers to a signal value calibrated by the analyte-insusceptible signal value. The calibrated data set may be provided by using the calibrated signal value for the signal value of the data set.

Alternatively, the calibrated data set may be provided by using the calibrated signal value and its further modification. For instance, the signal value calibrated by the analyte-insusceptible signal value may be further calibrated by adding or subtracting a certain value to or from the calibrated signal value. Particularly, the signal value calibrated by the analyte-insusceptible signal value may be further calibrated by subtracting baseline signal value.

According to an embodiment, the calibrated data set in the step (b) may be provided by using the calibrated signal values for the signal value of the data set with further modification.

According to an embodiment, the method further comprises the step of performing the signal-generating process to obtain a data set of the target analyte in the sample before the step (a).

According to an embodiment, the data set of the target analyte may have information indicating the presence or absence of the target analyte in the sample. In this case, the method provided by the present invention is described as “a method for calibrating data set representing the presence or absence of a target analyte in a sample”. The calibration of a data set representing the presence or absence of a target analyte in a sample is performed eventually for determining the presence or absence of a target analyte in a sample. The term used “determining the presence or absence of an analyte in a sample” means determining qualitatively or quantitatively the presence or absence of an analyte in a sample.

According to an embodiment, the calibrated data set is used for qualitative or quantitative detection of the target analyte in the sample. Qualitative detection refers to analyses in which substances are identified or classified on the basis of their chemical or physical properties, such as chemical reactivity, solubility, molecular weight, melting point, radiative properties (emission, absorption), mass spectra, nuclear half-life, etc. Quantitative detection refers to analyses in which the amount or concentration of an analyte may be determined (estimated) and expressed as a numerical value in appropriate units.

II. Method for Calibrating a Data Set of a Target Analyte Using Analyte-Insusceptible Signal Value Provided by a Data Set Obtained from a Sample to be Analyzed

The analyte-insusceptible signal value may be provided by a data set which is obtained from a signal-generating process for a sample to be analyzed. In this embodiment, the data set may be calibrated much more accurately because a signal value used for the calibration of the data set is directly obtained from the reaction performed for the sample to be analyzed.

Signal values of data sets from signal-generating processes may be varied depending on the amount of a target analyte in a sample. In an example of a real-time PCR, if a target nucleic acid is absent in a sample, the data set having almost identical signal values over the whole cycles may be obtained. Contrarily, if the target nucleic acid is present in the sample, the data set having signal values reflecting the presence or amount of the target nucleic acid may be obtained.

Meanwhile, even when a target analyte is present in a sample, signal values in a region of a data set are likely to be little or no affected by the presence or the amount of the target analyte in the sample. For example, signal value levels of a nucleic acid amplification reaction are very similar in a background region of a data set between the positive sample and the negative sample. The signal values in the background region of the data set are little or no affected by the presence/absence or the amount of the target nucleic acid. Accordingly, the signal value difference in background regions between a plurality of data sets may reflect the inter-instrument variations or inter-well variations within an instrument.

In one aspect of this invention, there is provided a method for calibrating a data set of a target analyte in a sample, which comprises:

(a) providing a background-representing signal value of the data set for calibrating the data set; wherein the data set is obtained from a signal-generating process for the target analyte using a signal-generating means; wherein the data set comprises a plurality of data points comprising cycles of the signal-generating process and signal values at the cycles;

wherein the background-representing signal value is provided by a signal value at a reference cycle of the data set and the reference cycle is selected within a background region of the data set where a signal generation is insusceptible to the presence or absence of the target analyte in the sample; and

(b) providing a calibrated data set by obtaining calibrated signal values by applying the background-representing signal value to the signal values of the data set.

### Step (a): Providing an Background-Representing Signal Value for Calibrating a Data Set

According to an embodiment, the analyte-insusceptible signal value may be provided by using a background-representing signal value of the data set. According to an embodiment, the background-representing signal value is provided by a signal value at a reference cycle of the data set and the reference cycle is selected within a background region of the data set where a signal is generated independently from the presence or absence of the target analyte in the sample.

The term “background-representing signal value” used herein means a representative signal value indicating a basal signal level of a data set. A high level of a background-representing signal value comes from a data set having an overall high basal signal level and a low level of a background-representing signal value results from an overall low basal signal level. Accordingly, where a plurality of data sets is calibrated by using their background-representing signal values, the variations in the signal values between the data sets become reduced.

The background-representing signal value may be calculated by various methods. The background-representing signal value is a signal value determined independently from the presence or absence of the target analyte.

According to an embodiment, the background-representing signal value may be determined by a signal value at a specific cycle. The specific cycle may be referred to as a reference cycle. The specific cycle may be a cycle that represents a basal signal level of the data sets. Particularly, it may be selected from cycles in a background region of an amplification reaction.

For applying to a plurality of data sets, the background-representing signal value is particularly determined based on an identical reference. The signal variations between the data sets can be reduced by the calibration using the background-representing signal value which is determined based on an identical reference.

The term “reference” means a benchmark for determining a background-representing signal value of a data set. The reference may be determined in such a manner that the background-representing signal value determined based on the reference indicates a basal signal level of the data set. The reference may be a cycle of a specific cycle number (e.g., 3 cycle or 5 cycle). Particularly, the reference may be a cycle or a region of cycles that has insignificant difference in signal values between its preceding and following cycles.

According to an embodiment, the background-representing signal value may be provided by a signal value at a reference cycle of a data set.

The reference cycle is selected from the cycles of the data set. The reference cycle is a cycle selected for determining a representative signal value used for calibrating signal values of the plurality of data points.

The reference cycle may include a reference temperature, a reference concentration or a reference time depending on meaning of a cycle. For instance, a reference temperature may be a reference cycle of a melting curve data set where the unit of cycle is temperature. In an embodiment of a melting data set, the terms “reference cycle” and “reference temperature” may be used interchangeably.

According to an embodiment, the reference cycle may be selected from the background region in which the generation of the signal is insusceptible to the presence or absence of the target analyte in a sample.

According to an embodiment, when the data set comprises a plurality of data sets from signal-generating processes, the reference cycle may be selected from a reference cycle group of each data set wherein the reference cycle group of each data set is provided in the same manner to each other.

According to an embodiment, when the data set comprises a plurality of data sets from signal-generating processes, the reference cycle may be selected from a reference cycle group of each data set wherein the reference cycle group is generated based on an identical rule. The identical rule may be applied equally to the determination of the reference cycle in all data sets.

The signal-generating process may comprise a plurality of signal-generating processes for the same-typed target analyte performed in different reaction vessels, and the data set may comprise a plurality of data sets obtained from the plurality of signal-generating processes. When the data set is a plurality of data sets, the reference cycle group of each data set is provided in the same manner to each other. The reference cycle group may be determined by various approaches. For example, the reference cycle group may comprise the cycles at which a similar level of signal values is measured. The reference cycle group may comprise the cycles at which a substantially identical level of signal values is measured before an amplification region. The reference cycle group may comprise the cycles where the coefficient of variation of signal values is within 5%, 6%, 7%, 8%, 9%, 10%.

According to an embodiment, the reference cycle may be selected from a reference cycle group of each data set, wherein the reference cycle group of each data set is provided in the same manner to each other. The plurality of data sets may be calibrated by using a reference cycle or cycles selected from a reference cycle group which is provided in the same manner (a common rule or a pre-determined criterion) to each other. The reference cycle selected for each of the plurality of data sets may be same to each other or different from each other.

The reference cycle group may comprise a single cycle wherein the number of the single cycle of each data set is identical to one another. According to an embodiment, an identical reference cycle may be provided for calibrating each data set of the plurality of data sets. When the data set comprises the plurality of data sets, an identical reference cycle is applied to a plurality of data sets to be analyzed with regard to an identical criterion. According to an embodiment, the plurality of data sets is calibrated by using an identical reference cycle.

The signal variation of data sets used for intra or inter-comparison analysis or analyzed by the identical criterion such as the same threshold need to be minimized. A range of data sets to be analyzed with regard to an identical criterion may be determined by a purpose of analysis, such as, but not limited thereto, a plurality of data sets obtained from a target analyte, obtained from the same type of sample, or obtained by the same reaction mixture (e.g, the same fluorescent molecules or same probe) may be analyzed with regard to an identical criterion.

However, according to an embodiment, when the data set comprises a plurality of data sets, at least two data sets of the plurality of data sets are applied with different reference cycles from each other so long as the signal values of the different reference cycles are substantially identical.

The reference cycle may be a pre-determined cycle or may be determined by an experiment. The reference cycle may be selected from cycles of a data set. Specifically, the reference cycle is selected from cycles in a region of a data set where amplification of signal is not sufficiently detected.

For example, when the data set is obtained by a nucleic acid amplification, process, it is preferable that the reference cycle is selected within a background signal region. The background region refers to an early stage of a signal-generating process before amplification of signal is sufficiently detected.

The background region may be determined by various approaches. For instance, the end-point cycle of the background region may be determined with a cycle of the first data point having a slope more than a certain threshold in the first derivatives of the data set obtained by a nucleic acid amplification process. Alternatively, the start-point cycle of the background region may be determined with a starting cycle of the first peak in the first derivatives of the data set obtained by a nucleic acid amplification process. Otherwise, the end-point cycle of the background region may be determined with a cycle of a data point having a maximum curvature.

According to an embodiment, the amplification process of the signal value may be a process providing signal values of a background signal region and a signal amplification region and the reference cycle may be selected within the background signal region. More specifically, according to an embodiment, the signal-generating process may be a polymerase chain reaction (PCR) or a real-time polymerase chain reaction (real-time PCR) and the reference cycle may be selected within the background signal region before a signal amplification region of the polymerase chain reaction (PCR) or the real-time polymerase chain reaction (real-time PCR). The signal values of initial background region of data sets obtained by a plurality of PCRs or real-time PCRs using the same target analyte under the same reaction condition would have theoretically the same or at least similar value, because the signal values of initial background region may comprise very low level of the signal value generated by target analyte regardless of the concentration of the target analyte. Therefore, it is preferable that the reference cycle is selected within the background signal region.

Therefore, the number of the reference cycles may be not more than 50, 40, 30, 25, 20, 19, 18, 17, 16, 15, 14, 13, 12, 11, 10, 9 or 8. The reference cycle of the present invention may be selected with avoiding an initial noise signal. The number of the reference cycles may be not less than 0, 1, 2, 3, 4, 5, 6 or 7. Particularly, the reference cycle of the present invention may be determined from cycles 1-30, 2-30, 2-20, 2-15, 2-10, 2-8, 3-30, 3-20, 3-15, 3-10, 3-9, 3-8, 4-8, or 5-8 in the background region.

According to an embodiment, the reference cycle may be a single reference cycle. A single cycle may be used as a reference cycle, and a signal value at the reference cycle of a data set may be used for providing a background-representing signal value. Alternatively, according to an embodiment, the reference cycle may comprise at least two reference cycles.

The reference cycle may comprise at least two reference cycles and the signal values at the cycles of the data set corresponding to the reference cycles may comprise at least two signal values.

A background-representative signal value for calibration may be provided by using a signal value which is calculated from the respective signal values at the cycles of the data set corresponding to the at least two reference cycles. For example, 4^{th}, 5^{th} and 6^{th} cycles may be designated as reference cycles, and the average of the signal values of 4^{th}, 5^{th} and 6^{th} cycles of a data set may be used for providing a background-representative signal value.

According to an embodiment, when a reference cycle is selected within a range of cycles of a data set, the reference cycle is selected from the cycles at which the signal values of the data sets to be analyzed with regard to an identical criterion would have the same value or at least similar value at the reference cycle.

According to an embodiment, the background-representing signal values of a plurality of data sets are provided by using an identical reference cycle. The background-representing signal value applied to each data set may be independently determined using an identical reference cycle. When the respective background-representing signal values are calculated from a plurality of data sets by using an identical reference cycle, the variations in the background-representing signal values between a plurality of data sets reflect signal value variations between the plurality of data sets. Accordingly, the signal variations between the plurality of data sets become reduced if the data sets are calibrated by using the background-representing signal values.

According to an embodiment, the data set of the step (a) may be a modified data set. According to an embodiment, the data set may be a modified data set of a raw data set. The modification may be mathematical modification. The modification may be performed by various methods depending on the purpose of the analysis. For example, the modification may be a removal of a specific amount of signal value or a removal of a blank signal.

According to an embodiment, the data set of the step (a) is a data set of which a blank signal value is removed. According to an embodiment, the blank signal value is a signal value obtained by no use of the signal-generating means.

### Step (b): Providing a Calibrated Data Set by Obtaining Calibrated Signal Values by Applying the Background-Representing Signal Value to the Signal Values of the Data Set

A calibrated data set may be provided by obtaining calibrated signal values by applying the background-representing signal value to the signal values of the data set. Particularly, a calibrated data set may be provided by obtaining calibrated signal values by applying the background-representing signal value to the signal values of the plurality of data points of the data set.

According to an embodiment, the calibrated data set is provided by obtaining calibrated signal values by dividing the signal values of the data set by the background-representing signal value.

According to an embodiment, the calibrated signal value is obtained by using the following mathematical equation 2:

Calibrated signal value=signal value/background-representing signal value Equation 2

The signal value in Equation 2 is an uncalibrated signal value.

The uncalibrated signal value may be a measured signal value or a processed signal value of the measured signal value. The process may be a process performed independently from a calibration process using the background-representing signal value. For example, the signal value processing may be performed by adding or subtracting a certain amount of value to or from the signal value. Particularly, the process may be removing a blank signal in whole or in part from the measured signal value.

The calibrated data set may be provided by the calibrated signal value for the signal value of the data set.

Alternatively, the calibrated data set may be provided by the calibrated signal value for the signal value of the data set with further modification.

Alternatively, the calibrated signal value is obtained by applying the background-representing signal value with further modification.

Where the data set comprise a plurality of data sets, a further modification may be applied to the plurality of data sets in the same manner to each other.

III. Method for Calibrating a Data Set of a Target Analyte Using Analyte-Insusceptible Signal Value Provided by a Data Set Obtained from a Separate Material

The analyte-insusceptible signal value may be provided by a data set obtained from a separate material not from a sample to be analyzed. When the analyte-insusceptible signal value is obtained from a separate material not from a sample to be analyzed, it is possible to prevent the effect of the analyte on the analyte-insusceptible signal value because the signal-generating process for calculating the analyte-insusceptible signal value is performed on a separate reaction vessel different from the vessel on which the signal-generating process for the sample is performed.

The separate material may be any material as long as it can generate a signal that reflects the reaction environment of each reaction. The separate material may be the same material as a target analyte in a sample, or it may be a different material from a target analyte in a sample.

The respective data sets obtained from the different reaction vessels (e.g., different instruments) can be calibrated with a substantially identical standard material in the following ways: First, the data sets are obtained from the signal-generating processes performed in the respective reaction vessels, and data sets for providing analyte-insusceptible signal values are obtained from the signal-generating processes performed in the respective reaction vessels using a substantially identical standard material. Then, the data sets from the respective reaction vessels are calibrated by applying the analyte-insusceptible signal value provided by the afore-mentioned data sets obtained using an identical standard material.

In one aspect of this invention, there is provided a method for calibrating a data set of a target analyte in a sample comprising:

(a) providing an analyte-insusceptible signal value for calibrating the data set; wherein the data set is obtained from a signal-generating process for the target analyte using a signal-generating means; wherein the data set comprises a plurality of data points comprising cycles of the signal-generating process and signal values at the cycles; wherein the analyte-insusceptible signal value is provided by a total signal change value of a standard data set; wherein the standard data set is obtained by a signal-generating process for a standard material of a target analyte; and

(b) providing a calibrated data set by obtaining calibrated signal values by applying the analyte-insusceptible signal value to the signal values of the data set.

The analyte-insusceptible signal value may be provided by a total signal change value of a standard data set in various approaches. For instance, the total signal change value of a standard data set per se may be designated as an analyte-insusceptible signal value; alternatively, a modified value of the total signal change value may be designated as an analyte-insusceptible signal value. According to an embodiment, the analyte-insusceptible signal value may be the total signal change value of the standard data set obtained by the signal-generating process for a standard material of the target analyte.

Accordingly, in another aspect of this invention, there is provided a method for calibrating a data set of a target analyte in a sample comprising:

(a) providing a total signal change value of a standard data set for calibrating the data set; wherein the data set is obtained from a signal-generating process for the target analyte using a signal-generating means; wherein the data set comprises a plurality of data points comprising cycles and signal values of the signal-generating process; wherein the standard data set is obtained by a signal-generating process for a standard material of a target analyte; and

(b) providing a calibrated data set by obtaining calibrated signal values by applying the total signal change value of the standard data set to the signal values of the data set.

Step (a): Providing a total signal change value of a standard data set for calibrating a data set

According to the present disclosure, a total signal change value of a standard data set for calibrating the data set is provided. The standard data set is obtained by a signal-generating process for a standard material of a target analyte.

The term “standard material” used herein refers to a material capable of providing a standard data set. The standard data set refers to a data set for providing an analyte-insusceptible signal value obtained from the signal-generating process using the standard material.

The standard material may be any material that generates the substantially same level of signal values whenever it is applied to signal-generating processes performed under the same reaction conditions. The standard material may be the same-typed material as the target analyte. Particularly, the standard materials applied to the plurality of reaction vessels may have the same concentration. It is particular that the standard material is in known concentration; however the concentration of the standard material does not need to be known so long as the same concentration of a standard material may be applied to a plurality of reactions. According to an embodiment, the standard data set may be a data set obtained by performing a signal-generating process for a target analyte of known concentration.

The standard data set refers to a data set obtained by using the standard material of a target analyte. The standard data set is a data set that reflects the reaction environment in which the target analyte in a sample is analyzed. According to an embodiment, the standard data set is obtained by using a reaction site which is identical to that used for obtaining the data set from the signal-generating process for the target analyte.

The reaction site is a physical space in which a process or a reaction is performed. The reaction site may include an instrument (e.g., PCR instrument) and a portion of an instrument (e.g., reaction well configured in PCR instrument). The reaction site is configured for target analyte detection reaction e.g., PCR amplification.

The standard data set may be obtained using an identical well(s) or instrument to that for obtaining the data set from the signal-generating process for the target analyte.

According to an embodiment, the standard data set may be obtained separately from a data set of a target analyte to be calibrated.

Where a standard data set may be obtained under the same reaction conditions as those of a data set of a target analyte in a sample, a signal-generating process for the standard data set is not necessarily performed together with a signal-generating process for the data set of a target analyte in a sample.

According to another embodiment, the standard data set may be obtained together with the data set of the target analyte to be calibrated. The standard data set may be obtained by a reaction performed under the same reaction conditions as reaction conditions under which a data set of a target analyte in a sample is obtained. A fine calibration for compensating a signal variation found in each instrument run may be achieved.

The common factors may be those which theoretically have constant values when an identical signal-generating process is performed under an identical condition. The common factor may be, for instance, a total signal change value (TSC), a cycle number of a first data point having a signal value more than a threshold or a signal value at a cycle in baseline region.

According to an embodiment, the data set may be calibrated by using a total signal change value as one of the common factor. The term used herein “total signal change value” means a signal change amount (increased or decreased) of a data set. The total signal change value may be a signal change amount (increased or decreased) of an entire data set or may be a signal change amount (increased or decreased) of a partial region of a data set. For example, the total signal change value may be a signal change value at the cycle having the greatest signal change value. Particularly, the total signal change value may be a difference between a signal value of baseline and a maximum signal value of the data set or a difference between a signal value of baseline and a signal value of the last cycle of the data set.

Meanwhile, when the total signal change value is determined within a region of the data set, the total signal change value may be a difference between the first cycle and the last cycle of the region of a data set or a difference between the maximum signal value and the minimum signal value of the region of a data set. Constant total signal change values may be obtained theoretically from the same or different instruments when signal-generating processes are performed using a standard material of a target analyte under an identical condition. Therefore, the calibration based on the total signal change value may reduce a variation between a plurality of the data sets.

The total signal change value (TSC) is the difference between the signal value of the background region and that of the amplified product. When a plurality of signal-generating processes using the standard material is performed under the same reaction environment (e.g., the same amount of primer or probe, etc.), the TSC may have the same value in spite of the existence of a difference in the concentration of the standard material.

Therefore, when a plurality of data sets is calibrated by the TSC, the standard material need not be of exactly the same concentration.

According to an embodiment, the signal-generating process may comprise a plurality of signal-generating processes for the same-typed target analyte performed in different reaction vessels, and the data set may comprise a plurality of data sets obtained from the plurality of signal-generating processes.

The plurality of data sets may be obtained by using a plurality of samples. Samples in the plurality of samples may be different from each other, particularly, at least two samples in the plurality of samples may be different from each other. Alternatively, Samples in the plurality of samples may be identical to each other.

According to an embodiment, an individual standard data set is provided for each data set of the plurality of the data sets and an individual total signal change value is provided. The individual standard data set is obtained by an individual signal-generating process for, an identical standard material of a target analyte.

When the data set for the target analyte in a sample comprises a plurality of data sets, an individual standard data set may be provided to each data set of the plurality of the data sets for the calibration of the plurality of data sets. The individual standard data set may be obtained by using a standard material of a target analyte under the same reaction conditions as those for each data set of the plurality of data sets.

The standard material used to provide each standard data set for a plurality of data sets may be the identical material.

An individual standard data set may be obtained by using an identical standard material and each of the plurality of data sets may be calibrated by using an individual total signal change value of the individual standard data set for each of the plurality of the data sets and thereby the variation between a plurality of data sets can be reduced. Each of a plurality of data sets may be calibrated by a total signal change value of a standard data set; wherein the standard data set is obtained by a signal-generating process for a standard material of a target analyte.

The total signal change values (TSCs) for calibrating the plurality of data sets may have different values from each other depending on reaction conditions of each data set. According to an embodiment, when the data set comprises at least two data sets, the at least two TSCs for the at least two data sets may have different TSCs from each other.

However, among the plurality of data sets, the data sets obtained by signal-generating processes performed under an identical reaction condition may be calibrated by an identical total signal change value. Accordingly, the plurality of data sets may be classified into at least two groups of data sets and an individual total signal change value is provided for calibrating each group of the data sets.

According to an embodiment, the plurality of data sets is classified into at least two groups of data set(s) and an individual total signal change value is provided for calibrating each group of data set(s); wherein the each group of data set(s) comprises one or more data sets.

The plurality of data sets may be classified into the same group by various criteria. According to an embodiment, the data sets obtained by the same reaction environment may be classified into the same group. For example, the data sets obtained from the same reaction vessel or the same instrument, or the data sets obtained by signal-generating processes with the reagents of the same batch number may be classified into the same group. The data sets belonging to the same group may be calibrated by the same TSC, and the data sets belonging to different groups may calibrated by different TSCs.

According to an embodiment, a total signal change value of a representative standard data set is provided identically for calibrating each of the plurality of the data sets.

According to an embodiment, a representative standard data set may be provided for the plurality of the data sets. When the data set is a plurality of data sets, a single representative standard data set may be applied to the plurality of the data sets. According to an embodiment, the plurality of data sets may be calibrated by using a single representative standard data set.

A group of data sets obtained by signal-generating processes performed under the same reaction conditions may be calibrated by using an identical standard data set, i.e. a representative standard data set. The representative standard data set may be different from a standard data set that is obtained from other signal-generating process performed under different reaction conditions, and the different standard data set is applied to another group of data sets. Accordingly, the variation between different groups of data sets can be reduced by applying each standard data set to each corresponding group of data sets.

The time and efforts to provide each standard data set for data sets belonging to the same group can be avoided by applying a single representative standard data set to all of the data sets of the same group for calibrating the data sets.

According to an embodiment, the data set of the step (a) may be a modified data set. According to an embodiment, the data set may be a modified data set of a raw data set. The modification may be mathematical modification.

The modification may be performed by various methods depending on the purpose of the analysis. For example, the modification may be a removal of the specific amount of signal value or a removal of a blank signal.

According to an embodiment, the data set of the step (a) is a data set of which a blank signal value is removed. According to an embodiment, the blank signal value is a signal value obtained by no use of the signal-generating means.

### Step (b): Providing a Calibrated Data Set by Obtaining Calibrated Signal Values by Applying the Total Signal Change Value of the Standard Data Set to the Signal Values of the Data Set

A calibrated data set may be provided by obtaining calibrated signal values by applying the total signal change value of the standard data set to the signal values of the data set. Particularly, a calibrated data set may be provided by obtaining calibrated signal values by applying the total signal change value of the standard data set to the signal values of the plurality of data points of the data set.

According to an embodiment, the calibrated data set is provided by obtaining calibrated signal values by dividing the signal values of the data set by the total signal change value of the standard data set.

According to an embodiment, the calibrated signal value is obtained by using the following mathematical equation 3:

Calibrated signal value=signal value/total signal change value of the standard data set Equation 3

The signal value in Equation 3 is an uncalibrated signal value.

The uncalibrated signal value may be a measured signal value or a processed signal value of the measured signal value. The process may be a process performed independently from a calibration process using the total signal change value of the standard data set. For example, the signal value processing may be performed by adding or subtracting a certain amount of value to or from the signal value. Particularly, the process may be removing a blank signal in whole or in part from the measured signal value.

The calibrated data set may be provided by using the calibrated signal value for the signal value of the data set.

Alternatively, the calibrated data set may be provided by using the calibrated signal value for the signal value of the data set with further modification. For instance, the signal value calibrated by the total signal change value of the standard data set may be further calibrated by adding or subtracting a certain amount of value to or from the calibrated signal value.

Alternatively, the signal value calibrated by the total signal change value of the standard data set may be further calibrated by multiplying or dividing it by certain value. The certain value may be the reference total signal change value (R-TSC) which is described hereinunder in more detail. When the data set comprises a plurality of data sets, the certain value applied to each of the plurality of data sets may be an identical value.

According to an embodiment, the calibrated signal values may be provided by applying a calibration coefficient to the signal values of the data set; wherein the calibration coefficient is provided by defining a relationship between the total signal change value of the standard data set and a reference total signal change value; wherein the reference total signal change value is an arbitrarily determined value. According to an embodiment, the signal values of a data set may be calibrated by applying a calibration coefficient to the signal values of the data set.

The calibration coefficient is a value that is applied to a plurality of data points of the data set for calibration. The calibrated data set may be provided by applying the calibration coefficient to the signal values of the plurality of data points of the data set.

The calibration coefficient may be provided by defining a relationship between the total signal change value of the standard data set and a reference total signal change value. The relationship between the total signal change value of the standard data set and a reference total signal change value may be defined by various ways, for example, the relationship may be defined mathematically. Particularly, the relationship between the total signal change value of the standard data set and the reference total signal change value may be a difference between the total signal change value of the standard data set and the reference total signal change value. More particularly, the difference between the total signal change value of the standard data set and the reference total signal change value may be a ratio of the total signal change value of the standard data set to the reference total signal change value.

A reference total signal change value refers to a total signal change value used in determining a calibration coefficient in comparison with a total signal change value of a standard data set.

According to an embodiment, the reference total signal change value may be determined by one or more data sets comprising a data set obtained from a signal-generating process using the reaction site which is different from that used for obtaining the data set from the signal-generating process for the target analyte.

Specifically, the reference total signal change value may be determined by the data set obtained from a signal-generating process using the reaction site which is different from that used for obtaining the data set from the signal-generating process for the target analyte together with the data set obtained from a signal-generating process using the reaction site which is identical to that used for obtaining the data set from the signal-generating process for the target analyte.

More specifically, the reference total signal change value may be determined by the data set obtained from a signal-generating process using the reaction site which is different from that used for obtaining the data set from the signal-generating process for the target analyte.

According to an embodiment, the reference total signal change value may be a pre-determined total signal change value. The reference total signal change value may be obtained by using a reference (or standard) vessel(s) or instrument(s) with substantially identical standard material used for obtaining the total change value of the standard data set.

The reference total signal change value of the present invention may be determined by a data set obtained from a signal-generating process for a standard material of a target analyte. Alternatively, the reference total signal change value of the present invention may be calculated from total signal change values of a plurality of data sets obtained from a plurality of signal-generating processes for a standard material of a target analyte. In this case, the reference total signal change value is an average or median value of a plurality of data sets obtained from a plurality of signal-generating processes for a standard material of a target analyte or may be predetermined by an experimenter based on the results of a plurality of signal-generating processes for a standard material of a target analyte.

Alternatively, according to an embodiment, when the standard data set is a plurality of standard data sets, the reference total signal change value may be determined from the plurality of standard data sets. For instance, one of the total signal change values of the plurality of standard data sets may be determined as a reference total signal change value. Alternatively, an average or median value of the total signal change values of the plurality of standard data sets may be determined as a reference total signal change value.

Accordingly, in another aspect of this invention, there is provided a method for calibrating a data set of a target analyte in a sample, which comprises:

(a) providing a calibration coefficient for calibrating the data set; wherein the data set is obtained from a signal-generating process for the target analyte using a signal-generating means; wherein the data set comprises a plurality of data points comprising cycles and signal values of the signal-generating process; wherein the calibration coefficient is provided by a ratio of (i) a total signal change value of a standard data set obtained by a signal-generating process for a standard material of a target analyte to (ii) a pre-determined reference total signal change value; and

(b) providing a calibrated data set by obtaining calibrated signal values by applying the calibration coefficient to the signal values of the data set.

A data set may be calibrated by reducing a blank signal value, followed by calibrating the blank signal-reduced data set by applying the calibration coefficient. In another aspect of this invention, there is provided a method for calibrating a data set of a target analyte in a sample, which comprises:

(a) providing a 1^{st} calibrated data set of which a blank signal value is reduced by reducing the blank signal value from the signal value of the data set; wherein the data set is obtained from a signal-generating process for the target analyte using a signal-generating means; wherein the data set comprises a plurality of data points comprising cycles and signal values of the signal-generating process;

(b) providing a calibration coefficient for calibrating the data set; wherein the calibration coefficient is provided by a ratio of (i) a total signal change value of a standard data set obtained by a signal-generating process for a standard material of a target analyte to (ii) the pre-determined reference total signal change value; and

(c) providing a 2^{nd} calibrated data set by obtaining calibrated signal values by applying the calibration coefficient to the signal values of the 1st calibrated data set.

According to an embodiment, the method further comprises the step of performing the signal-generating process to obtain a data set of the target analyte in the sample before the step (a).

According to an embodiment, the data set of the target analyte may have information indicating the presence or absence of the target analyte in the sample.

According to an embodiment, the calibrated data set is used for qualitative or quantitative detection of the target analyte in the sample.

### IV. Storage Medium, Device and Computer Program

In another aspect of this invention, there is provided a computer readable storage medium containing instructions to configure a processor to perform a method for calibrating a data set of a target analyte in a sample, the method comprising:

(a) providing an analyte-insusceptible signal value for calibrating the data set; wherein the data set is obtained from a signal-generating process for the target analyte using a signal-generating means; wherein the data set comprises a plurality of data points comprising cycles and signal values of the signal-generating process;

wherein the analyte-insusceptible signal value is provided (i) by a background-representing signal value of the data set; wherein the background-representing signal value is provided by a signal value at a reference cycle of the data set and the reference cycle is selected within a background region of the data set where signal generation is insusceptible by the presence or absence of the target analyte in the sample; or (ii) by a total signal change value of a standard data set; wherein the standard data set is obtained by a signal-generating process for a standard material of a target analyte; and

(b) providing a calibrated data set by obtaining calibrated signal values by applying the analyte-insusceptible signal value to the signal values of the data set.

In another aspect of this invention, there is provided a computer program to be stored on a computer readable storage medium to configure a processor to perform a method for calibrating a data set of a target analyte in a sample, the method comprising:

(a) providing an analyte-insusceptible signal value for calibrating the data set; wherein the data set is obtained from a signal-generating process for the target analyte using a signal-generating means; wherein the data set comprises a plurality of data points comprising cycles and signal values of the signal-generating process;

wherein the analyte-insusceptible signal value is provided (i) by a background-representing signal value of the data set; wherein the background-representing signal value is provided by a signal value at a reference cycle of the data set and the reference cycle is selected within a background region of the data set where signal generation is insusceptible by the presence or absence of the target analyte in the sample; or (ii) by a total signal change value of a standard data set; wherein the standard data set is obtained by a signal-generating process for a standard material of a target analyte; and

(b) providing a calibrated data set by obtaining calibrated signal values by applying the analyte-insusceptible signal value to the signal values of the data set.

The program instructions are operative, when performed by the processor, to cause the processor to perform the present method described above. The program instructions for performing the method for calibrating a data set of a target analyte in a sample may comprise an instruction to provide an analyte-insusceptible signal value for calibrating the data set; and an instruction to provide a calibrated data set by obtaining calibrated signal values by applying the analyte-insusceptible signal value to the signal values of the data set.

The present method described above is implemented in a processor, such as a processor in a stand-alone computer, a network attached computer or a data acquisition device such as a real-time PCR machine.

The types of the computer readable storage medium include various storage medium such as CD-R, CD-ROM, DVD, flash memory, floppy disk, hard drive, portable HDD, USB, magnetic tape, MINIDISC, nonvolatile memory card, EEPROM, optical disk, optical storage medium, RAM, ROM, system memory and web server.

The data set may be received through several mechanisms. For example, the data set may be acquired by a processor resident in a PCR data acquiring device. The data set may be provided to the processor in a real time as the data set is being collected, or it may be stored in a memory unit or buffer and provided to the processor after the experiment has been completed. Similarly, the data set may be provided to a separate system such as a desktop computer system via a network connection (e.g., LAN, VPN, intranet and Internet) or direct connection (e.g., USB or other direct wired or wireless connection) to the acquiring device, or provided on a portable medium such as a CD, DVD, floppy disk, portable HDD or the like to a stand-alone computer system. Similarly, the data set may be provided to a server system via a network connection (e.g., LAN, VPN, intranet, Internet and wireless communication network) to a client such as a notebook or a desktop computer system.

The instructions to configure the processor to perform the present invention may be included in a logic system. The instructions may be downloaded and stored in a memory module (e.g., hard drive or other memory such as a local or attached RAM or ROM), although the instructions can be provided on any software storage medium such as a portable HDD, USB, floppy disk, CD and DVD. A computer code for implementing the present invention may be implemented in a variety of coding languages such as C, C++, Java, Visual Basic, VBScript, JavaScript, Perl and XML. In addition, a variety of languages and protocols may be used in external and internal storage and transmission of data and commands according to the present invention.

In still further aspect of this invention, there is provided a device for calibrating data set of a target analyte in a sample, comprising (a) a computer processor and (b) the computer readable storage medium described above coupled to the computer processor.

According to an embodiment, the device further comprises a reaction vessel to accommodate the sample and signal-generating means, a temperature controlling means to control temperatures of the reaction vessel and/or a detector to detect signals at amplification cycles.

According to an embodiment, the computer processor permits not only to receive values of signals at cycles but also to analyze a sample or data set or obtain a calibrated data set of a target analyte in a sample. The processor may be prepared in such a manner that a single processor can do all performances described above. Alternatively, the processor unit may be prepared in such a manner that multiple processors do multiple performances, respectively.

According to an embodiment, the processor may be embodied by installing software into conventional devices for detection of target nucleic acid molecules (e.g. real-time PCR device).

The features and advantages of this invention will be summarized as follows:

(a) According to the present invention, a data set is calibrated conveniently by applying an analyte-insusceptible signal value to the data set such that the inter- and intra-instrument signal variations of data sets are reduced effectively. Particularly, not only the inter-instrument signal variations but also the intra-instrument signal variations are reduced by the present invention, whereby the data set is capable of being analyzed with a higher accuracy and reproducibility. The effects and results originated from the present invention urge us to reason that all types of signal variations between signal-generating processes in different reaction sites (e.g., different reaction vessels or different wells) can be largely and effectively reduced by a software-typed process.

(b) The calibration method of the present invention can be configured in software so that the method of the present invention is capable of being applied universally to various analytical instruments (e.g., real-time PCR instruments) regardless of manufacturers. Therefore, the method of the present invention is much more convenient and versatile than conventional hardware calibration methods.

(c) The signal variation is a serious problem in detecting RNA viruses using degenerated primers and/or probes. The signal variation between data sets can be reduced dramatically through the present invention. Therefore, the present invention can be an excellent solution for the signal variation caused by using degenerated primers and/or probes for detecting RNA viruses.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1: Calibration of Data Set by Signal Variation-Based Normalization (SVN) and Analysis of Calibrated Data Set

Methods of controlling the input or output signal intensity in hardware-wise have been widely used for minimizing the intra-instrument signal variations in a real-time PCR. For example, the output intensity of a light source (e.g., LED and Halogen lamp) is adjusted or the input intensity of signals is controlled through a filter of a detector for calibrating signals.

In Examples, the Signal Variation-based Normalization (SVN) method of the present disclosure was used for correcting variations in amplified signals of data sets. The SVN method was named for a process using a total signal change value of a standard data set as described above. The signal variations in the following three groups of data sets were compared and analyzed: (i) a group of data sets obtained from an instrument without a hardware adjustment; (ii) a group of data sets obtained from an instrument with a hardware adjustment; and (iii) a group of data sets software-wise calibrated by the SVN.

<1-1> Preparation of Data Set

A real-time PCR for a target nucleic acid molecule was performed using TaqMan probe as a signal-generating means with 50 cycles of an amplification on three CFX96™ Real-Time PCR Detection Systems (Bio-Rad) listed in Table 1. The target nucleic acid molecule was a genomic DNA of Ureaplasma urealyticum. The interactive dual label was provided by TaqMan probe labeled with a reporter molecule (FAM) and a quencher molecule (BHQ-1).

The reaction was conducted in the tube containing a target nucleic acid molecule, a downstream primer, an upstream primer, TaqMan probe, and Master Mix containing MgCl₂, dNTPs and Taq DNA polymerase. The tube containing the reaction mixture was placed on the real-time thermocycler (CFX96, Bio-Rad). The reaction mixture was denatured for 15 min at 95° C. and subjected to 50 cycles of 10 sec at 95° C., 60 sec at 60° C., 10 sec at 72° C. Detection of the signal was performed at 60° C. at each cycle.

Ninety-five (95) reactions in the respective 96-wells were carried out under the same condition in the respective instruments using the samples containing the same target nucleic acid of the same concentration. Further, one reaction was carried out simultaneously with the 95 reactions in a remaining well of the 96-wells in the respective instruments using a sample containing a target analyte of known concentration, which was utilized as standard reaction. By analyzing the data sets and standard data set obtained from the above reactions, the level of an inter-instrument or intra-instrument signal variation and the level of reduction in the signal variations by the SVN method were analyzed.

A total of six groups of raw data sets were prepared, including three groups of data sets obtained from the reactions in the instrument without a hardware adjustment and the other three groups of data sets obtained from the reactions in the instrument with a hardware adjustment. Each group includes 95 data sets and 1 standard data set obtained from the 96 well-reactions.

The baseline subtracted data sets were obtained from the raw data sets. The baseline subtracted data sets were prepared according to the following way. The baseline was established from the third cycle to the cycle just before the signal amplification occurrence and then a linear regression equation was calculated for the cycles in the established baseline region. The baseline subtracted data sets were prepared by subtracting the signal values calculated with the regression straight line equation at the corresponding cycle from the signal values measured at the respective cycles.

TABLE 1

| Instrument Number | Real-time PCR Instrument |
|---|---|
| Instrument 1 | CFX96 Real-time Cycler |
| Instrument 2 | (Bio-Rad) |
| Instrument 3 | |

<1-2> Analysis of Data Set Obtained from Instrument without Hardware Adjustment

The raw data sets and their baseline subtracted data sets obtained in Example <1-1> were used. The signal variations were analyzed for three groups of the raw data sets obtained from the instruments without a hardware adjustment and for three groups of the baseline subtracted data sets.

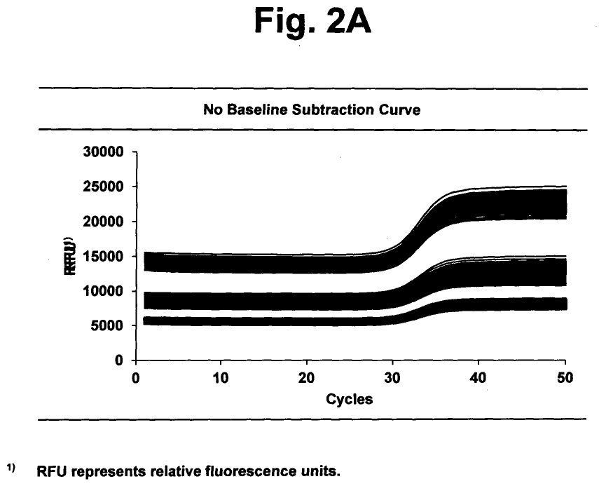

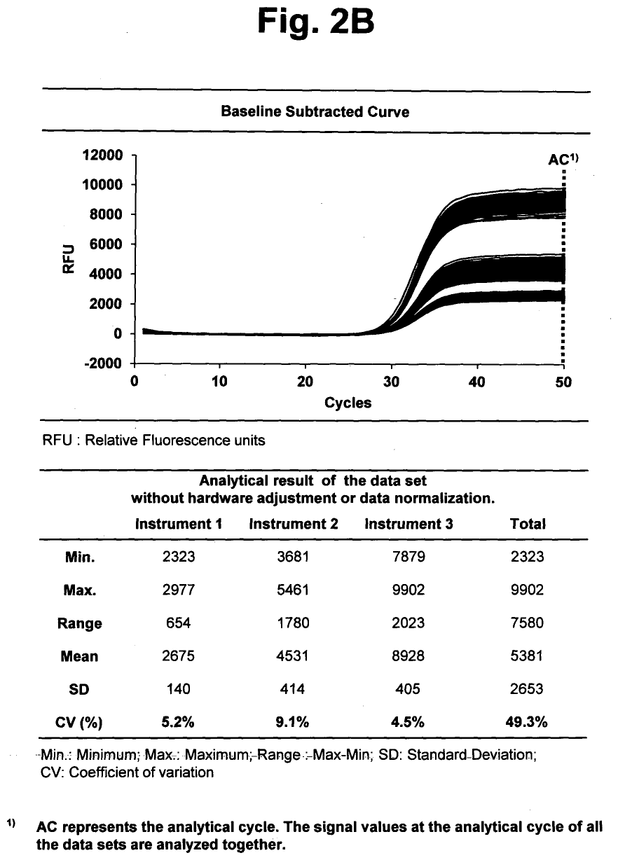

In order to compare the background signal intensities of three instruments, amplification curves without baseline subtraction (No Baseline Subtraction Curve) were obtained by plotting the raw data sets without baseline subtraction (FIG. 2A).

As shown in FIG. 2A, the background signals of the respective instruments were shown to be separated from each other, which is unlike to a theoretical expectation that background signals having the same intensities will be plotted for amplification reactions under the same condition.

In order to compare signal variations in the amplification region, the amplification curves with baseline subtraction (Baseline Subtracted Curve) were prepared by plotting the baseline subtracted data sets obtained from the respective three instruments.

The last cycle (i.e., 50^{th} cycle) of the baseline subtracted data sets was designated as an analytical cycle and the coefficient of variation (CV) of the amplification signals at 50^{th} cycle was calculated (FIG. 2B). The coefficient of variation (CV) is defined as the ratio of the standard deviation to the arithmetic mean for the data. The intra-instrument coefficient of variation was calculated from the standard deviation and the arithmetic mean of signal values at a specific cycle among the results of multiple reactions measured on a single instrument. The inter-instrument coefficient of variation was calculated from the standard deviation and the arithmetic mean of signal values at a specific cycle in the resulting data sets of all reactions measured in three instruments used in the experiments.

The coefficients of variations of the amplification signals at the last cycle of amplification curves with baseline subtraction were represented in FIG. 2B. The intra-instrument coefficients of variations of the amplification signals of the instruments 1, 2, and 3 were analyzed as 5.2%, 9.1%, and 4.5%, respectively and the inter-instrument coefficient of variation of the amplification signals of the instruments 1, 2, and 3 was analyzed as 49.3%.

<1-3> Analysis of Data Set Obtained from Instrument with Hardware Adjustment

The raw data sets and their baseline subtracted data sets obtained in Example <1-1> were used. The signal variations were analyzed for three groups of the raw data sets obtained from the instruments with a hardware adjustment and for three groups of the baseline subtracted data sets.

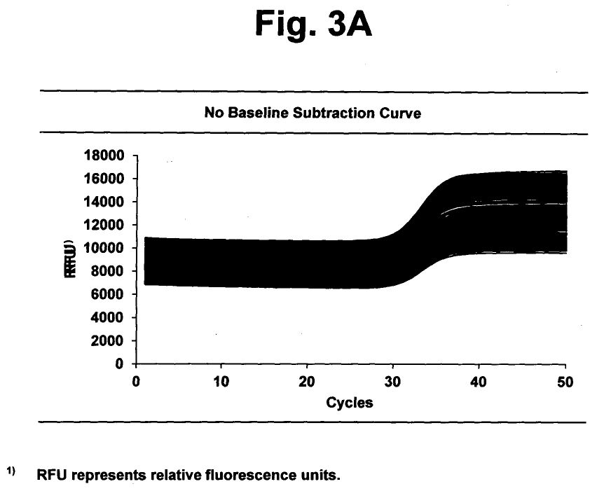

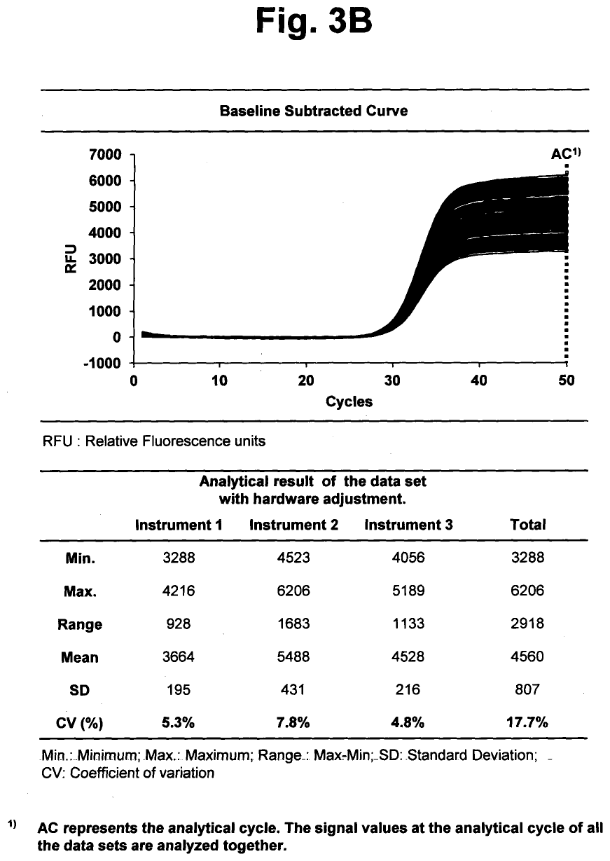

The amplification curves without baseline subtraction (No Baseline Subtraction Curve) were analyzed according to the same method as described in Example <1-2>. As shown in FIG. 3A, the inter-instrument background signal variations became reduced compared to the instruments without a hardware adjustment.

The coefficients of variations of the amplification signals at the last cycle of the amplification curves with baseline subtraction (Baseline Subtracted Curve) were analyzed with the same way as described in Example <1-2>. As shown in FIG. 3B, the intra-instrument coefficients of variations of the amplification signals of the instruments 1, 2, and 3 were analyzed as 5.3%, 7.8%, and 4.8%, respectively and the inter-instrument coefficient of variation of the amplification signals of the instruments 1, 2, and 3 was analyzed as 17.7%.

When the above results were compared with the results of the data sets obtained from the instrument without a hardware adjustment in Example <1-2>, it was proved that the inter-instrument coefficient of variation of the amplification signals was reduced by 31.6% P (percentage points) while there was negligent difference in the intra-instrument coefficient of variation of the amplification signals.

From the above results, it can be concluded that even though the calibration by the hardware adjustment can reduce partly the inter-instrument coefficient of variation of the amplification signals of the instruments, a considerable level of signal variations between the instruments still exists.

<1-4> Analysis of Data Set Software-Wise Calibrated Using SVN

The Signal Variation-based Normalization (SVN) is a method of proportionally normalizing the data sets using a total signal change value (TSC) of the standard data sets of each instrument and additionally using a reference total signal change value (R-TSC).

The total signal change value (TSC) means a signal change (increased or decreased) amount of a corresponding standard data set. The standard data set refers to a data set obtained through a signal-generating process for a target analyte of known concentration (standard concentration). The standard data set of each instrument is obtained by performing a signal-generating process on an instrument using a target analyte of known concentration.

The reference total signal change value (R-TSC) can be determined from a total signal change value of data sets obtained from a standard instrument or a total signal change value of a plurality of data sets. In addition, the reference total signal change value (R-TSC) can be determined by an experimenter based on results of a plurality of signal-generating processes for a corresponding target analyte.

In the present disclosure, the calibration effect of the raw data sets is achieved by applying the TSC. Moreover, the numerical values of signal intensities in the calibrated data sets can be further adjusted with the R-TSC.

As a result of calibrating the data sets using the SVN, it was verified that the inter-instrument signal variations were further reduced.

<1-4-1> Calibration of Data Set by SVN Using Total Signal Change Value

In this Example, the data sets were calibrated by the SVN applying the total signal change value (TSC) to the data sets obtained from each instrument. The raw data sets of six groups obtained in Example <1-1> were software-wise calibrated using the SVN according to the following steps:

<Step 1>

An instrument-specific standard data set was obtained by performing a standard signal-generating process using a target analyte of standard concentration under the same reaction condition as that of signal-generating processes performed for obtaining data sets from an experimental sample. A total signal change value was obtained from the standard data set.

In order to calculate the total signal change value of the standard data set, the baseline was subtracted from the obtained standard data set to yield a baseline subtracted data set as described in Example <1-1>. The total signal change value was calculated from the baseline subtracted data sets. The RFU at the last 50^{th} cycle (End-Point) of the baseline subtracted data set was designated as the total signal change value.

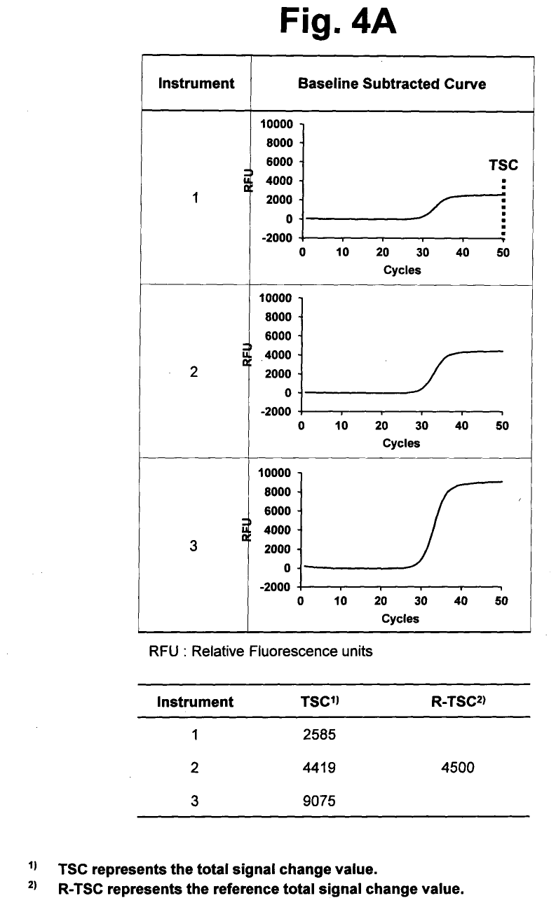

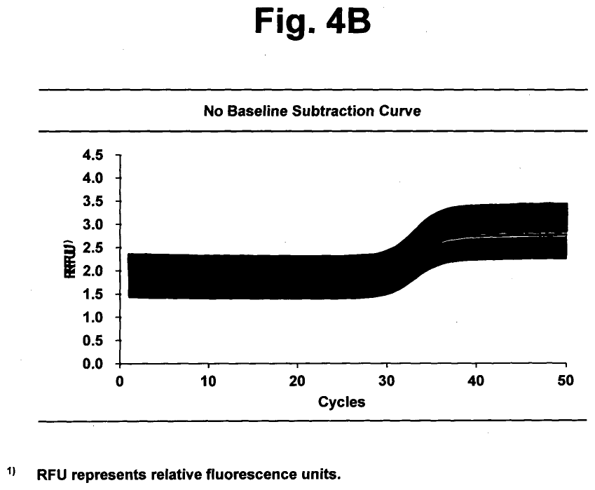

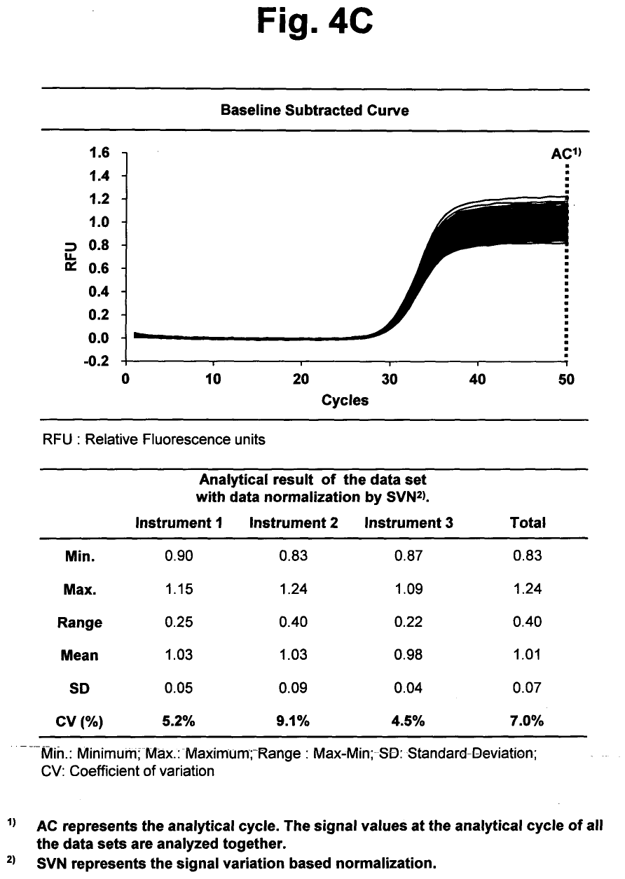

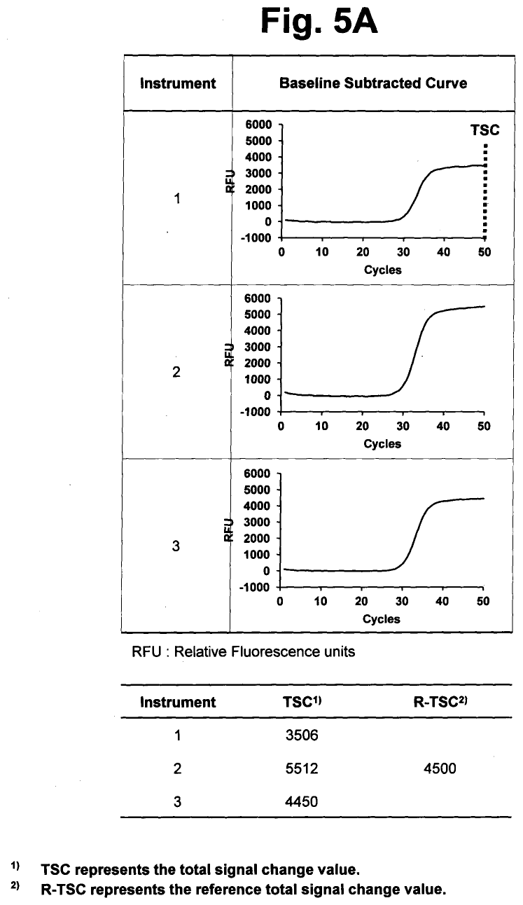

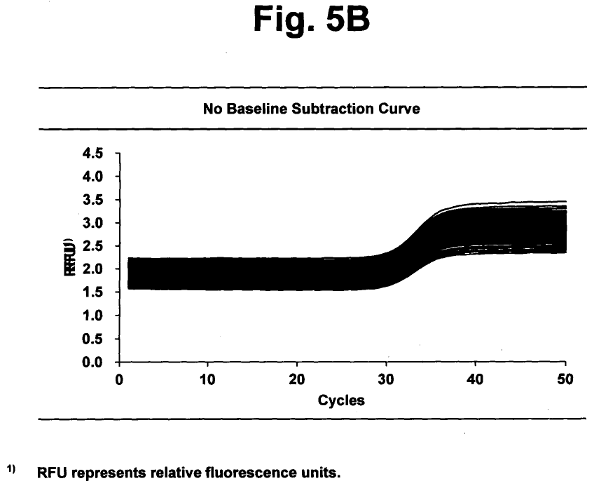

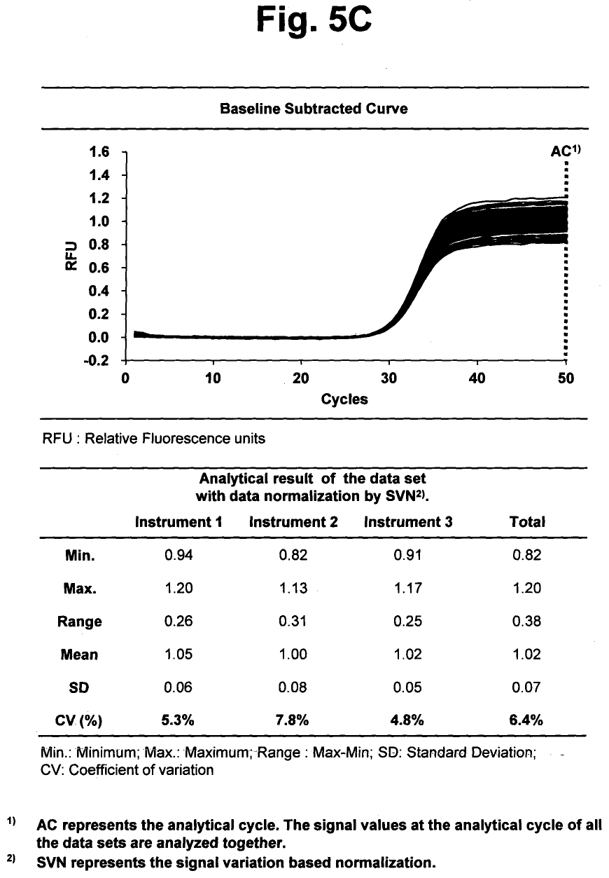

In this Example, the standard data sets were prepared from each instrument, and the instrument-specific total signal change values were calculated. The total signal change values (TSCs) of the standard data sets obtained from the instruments 1, 2, and 3 without or with a hardware adjustment were measured as shown in Table 2 (FIGS. 4A and 5A).

TABLE 2

Total Signal Change Values (TSCs) of
Standard Data Sets (RFU)

| Hardware Adjustment | − | + |
|---|---|---|
| Instrument 1 | 2585 | 3506 |
| Instrument 2 | 4419 | 5512 |
| Instrument 3 | 9075 | 4450 |

<Step 2>

The signal values at all cycles of the raw data sets obtained from each instrument were calibrated using the instrument-specific TSCs obtained from each instrument.

Calibrated Signal Value (RFU)=Signal Value of Raw Data Set (RFU)÷TSC

The calibrated six groups of data sets were obtained by calibrating the six groups of the raw data sets provided in Example <1-1> according to the above steps 1 to 2.

A. Analysis of the Results of Calibration of Data Sets Obtained from an Instrument without a Hardware Adjustment

The instrument-specific TSCs were calculated from the respective standard data sets obtained from the instruments 1, 2, and 3 without a hardware adjustment (FIG. 4A). The data sets obtained from the instruments 1, 2 and 3 without a hardware adjustment were calibrated by the SVN using the instrument-specific TSCs, and the resulting calibrated data sets were analyzed.

FIGS. 4B and 4C show the amplification curves (FIG. 4B) and the intra- and inter-instrument coefficients of variations (FIG. 4C) for the calibrated data sets which were provided by calibrating the data sets obtained from the instruments 1, 2 and 3 without a hardware adjustment through the steps 1 to 2.

The amplification curves were obtained by plotting the calibrated data sets. FIG. 4B shows the amplification curves provided by plotting the calibrated data sets without baseline subtraction (No Baseline Subtraction Curve), in which the intensities of the signals in the background and amplification regions can be compared. The signal values of the data sets from the three instruments were shown to be normalized. Specifically, the signals in the background region became similar to one another and the signals in the amplification region also became similar to one another.

In addition, the baseline subtracted amplification curves (Baseline Subtracted Curve) were obtained by subtracting the baseline from the calibrated data sets and plotting the baseline subtracted data sets, and then the coefficient of variation of the signals at the 50^{th} cycle was calculated. In FIG. 4C representing the baseline subtracted amplification curve, the signal variations in the amplification region were compared. The coefficients of variations of the amplification signals were analyzed. The intra-instrument coefficients of variations of the amplification signals were 5.2%, 9.1% and 4.5%, respectively and the inter-instrument coefficient of variation of the amplification signal was 7.0%.

The following three coefficients of variations were compared and analyzed: (i) the coefficient of variation of the signals in the data sets obtained from the instrument without a hardware adjustment in Example <1-2>; (ii) the coefficient of variation of the signals in the data sets obtained from the instrument with a hardware adjustment in Example <1-3>; and (iii) the coefficient of variation of the signals in the calibrated data sets provided by calibrating the data sets with the SVN using the TSC of this Example, in which the data sets had been obtained from the instrument without a hardware adjustment.

As shown in Table 3, the calibrated data sets by the SVN using the instrument-specific TSCs have following characteristics: When compared with the data sets obtained from the instrument without a hardware adjustment, the inter-instrument coefficient of variation of the amplification signal was remarkably reduced by 42.3% P (percentage points). In addition, when compared with the data sets obtained from the instrument with a hardware adjustment, the inter-instrument coefficient of variation of the amplification signals was significantly reduced by 10.7% P (percentage points).

It would be demonstrated that the signal calibration method of the SVN can effectively reduce the inter-instrument signal variations by using the total signal change values (TSCs) obtained from the instrument-specific standard data sets.

TABLE 3

| | | | | |
|---|---|---|---|---|
| Calibration | SVN Using the TSCs | − | − | + |
| Method | Hardware Adjustment | − | + | − |
| Result of | Instrument 1 | 5.2 | 5.3 | 5.3 |
| Analysis | Instrument 2 | 9.1 | 7.8 | 9.1 |
| of Amplification | Instrument 3 | 4.5 | 4.8 | 4.5 |
| Signal | Total | 49.3 | 17.7 | 7.0 |
| (Coefficient | | | | |
| of Variation, CV %) | | | | |

B. Analysis of the Results of Calibration of Data Sets Obtained from an Instrument with a Hardware Adjustment

The instrument-specific TSCs were calculated from the respective standard data sets obtained from the instruments 1, 2, and 3 with a hardware adjustment (FIG. 5A).

The data sets obtained from the instruments 1, 2 and 3 with a hardware adjustment were further calibrated by the SVN using the instrument-specific TSCs, and the resulting calibrated data sets were analyzed.

FIGS. 5B and 5C show the amplification curves (FIG. 5B) and the intra- and inter-instrument coefficients of variations (FIG. 5C) for the calibrated data sets which were provided by calibrating the data sets obtained from the instruments 1, 2 and 3 with a hardware adjustment through the steps 1 to 2.

The amplification curves were obtained by plotting the calibrated data sets. FIG. 5B shows the amplification curves without baseline subtraction (No Baseline Subtraction Curve) for the calibrated data sets, in which the signal intensities in the background and amplification regions can be compared. The signal values of the data sets from the respective three instruments were shown to be normalized. Specifically, the signals in the background region became similar to one another and the signals in the amplification region also became similar to one another.

In addition, the baseline subtracted amplification curves were obtained by subtracting the baseline from the calibrated data sets and plotting the baseline subtracted data sets, and then the coefficient of variation of the signals at the 50^{th} cycle was calculated.

In FIG. 5C representing the baseline subtracted curve (Baseline Subtracted Curve), the signal variations in the amplification region were compared. The coefficients of variation of the amplification signals were analyzed. The intra-instrument coefficients of variations of the amplification signals were 5.3%, 7.8% and 4.8%, respectively and the inter-instrument coefficient of variation of the amplification signals was 6.4%.

The following three coefficients of variations were compared and analyzed: (i) the coefficient of variation of the signals in the data sets obtained from the instrument without a hardware adjustment in Example <1-2>; (ii) the coefficient of variation of the signals in the data sets obtained from the instrument with a hardware adjustment in Example <1-3>; and (iii) the coefficient of variation of the signal in the calibrated data sets provided by calibrating the data sets by the SVN using the TSC of this Example, in which the data sets had been obtained from the instrument with a hardware adjustment.

As shown in Table 4, the calibrated data sets by the SVN using the instrument-specific TSCs have following characteristics: When compared with the data sets obtained from the instrument without a hardware adjustment, the inter-instrument coefficient of variation of the amplification signals was remarkably reduced by 42.9% P (percentage points). In addition, when compared with the data sets obtained from the instrument with a hardware adjustment, the inter-instrument coefficient of variation of the amplification signals was significantly reduced by 11.3% P (percentage points).

It would be demonstrated that the signal calibration method of the SVN can effectively reduce the inter-instrument signal variations by using total signal change values (TSCs) obtained from the instrument-specific standard data sets. Interestingly, data sets obtained from the instrument with a hardware adjustment can be further calibrated by the SVN, such that an inter-instrument variation can be more precisely corrected.

TABLE 4

| | | | | |
|---|---|---|---|---|
| Calibration | SVN Using the TSCs | − | − | + |
| Method | Hardware Adjustment | − | + | + |
| Result of | Instrument 1 | 5.2 | 5.3 | 5.3 |
| Analysis | Instrument 2 | 9.1 | 7.8 | 7.8 |
| of Amplification | Instrument 3 | 4.5 | 4.8 | 4.8 |
| Signal | Total | 49.3 | 17.7 | 6.4 |
| (Coefficient | | | | |
| of Variation, CV %) | | | | |

<1-4-2> Calibration of Data Set by SVN Using Total Signal Change Value and Reference Total Signal Change Value

In this Example, the data sets were calibrated by the SVN applying both the TSC and R-TSC to the data sets obtained from each instrument. The raw data sets of six groups obtained in Example <1-1> were software-wise calibrated using the SVN according to the following steps:

<Step 1>

As shown in Example <1-4-1>, the total signal change values (TSCs) were measured from the standard data sets (Table 2).

<Step 2>

The signal values at all cycles of the raw data sets obtained from each instrument were calibrated using the instrument-specific TSCs obtained from each instrument.

1^{st} Calibrated Signal Value (RFU)=Signal Value of Raw Data Set (RFU)÷TSC

<Step 3>

In this Example, the RFU 4500 was designated as the R-TSC (FIGS. 4A and 5A), which is similar to the mean of the total signal change values of the data sets obtained from three instruments with a hardware adjustment of Example <1-1> (FIG. 3B). The 2^{nd} calibrated signal values were obtained by applying the R-TSC to the 1^{st} calibrated signal values.

2^{nd} Calibrated Signal Value (RFU)=1^{st} Calibrated Signal Value (RFU)×R-TSC

The calibrated six groups of data sets were obtained by calibrating the six groups of the raw data sets provided in Example <1-1> according to the above steps 1 to 3.

A. Analysis of the Results of Calibration of Data Sets Obtained from an Instrument without a Hardware Adjustment

The data sets obtained from an instrument without a hardware adjustment were calibrated by the SVN using both the TSC and R-TSC, and the resulting calibrated data sets were analyzed. As a result, it was found that the variation of the signal values of the calibrated data sets were the same as those represented in FIG. 4B but the intensities of signals (Y-axis values) were different from those represented in FIG. 4B.

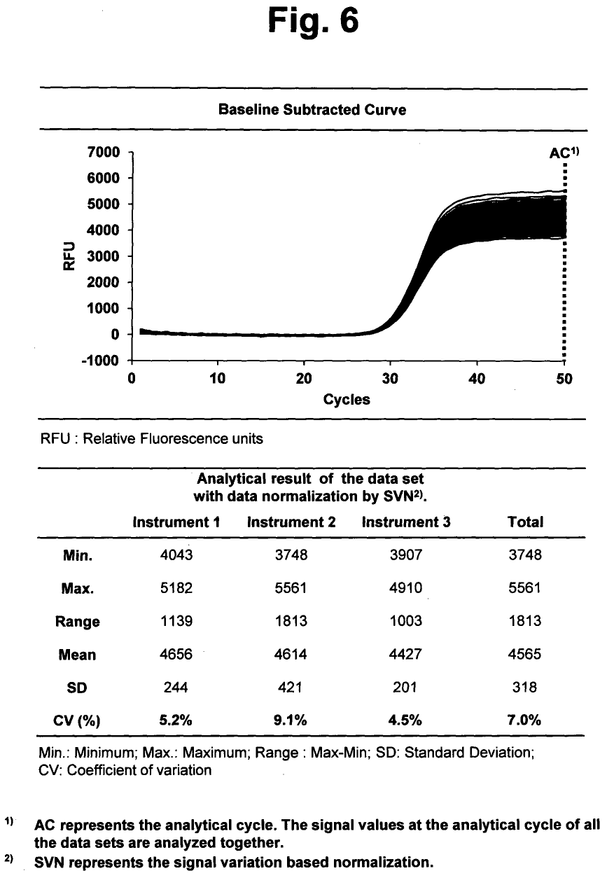

FIG. 6 shows the baseline subtracted amplification curves and the intra- and inter-instrument coefficients of variations for the calibrated data sets which were provided by calibrating the data sets obtained from the instrument without a hardware adjustment through the steps 1 to 3.

As shown in FIG. 6, the intra-instrument coefficients of variations of the amplification signals were 5.2%, 9.1% and 4.5%, respectively and the inter-instrument coefficient of variation of the amplification signal was 7.0%. It would be noticeable that the intra- and inter-instrument coefficients of variations of the normalized amplification signals of this Example were the same as those of Example <1-4-1> represented by FIG. 4C but their signal intensities (Y-axis values) were different from each other.

B. Analysis of the Results of Calibration of Data Sets Obtained from an Instrument with a Hardware Adjustment

The data sets obtained from an instrument with a hardware adjustment were calibrated by the SVN using both the TSC and R-TSC, and the resulting calibrated data sets were analyzed. As a result, it was found that the variations of the signal values of the calibrated data sets were the same as those of signals represented in FIG. 5B but the intensities of signals (Y-axis values) were different from those of the signals represented in FIG. 5B.

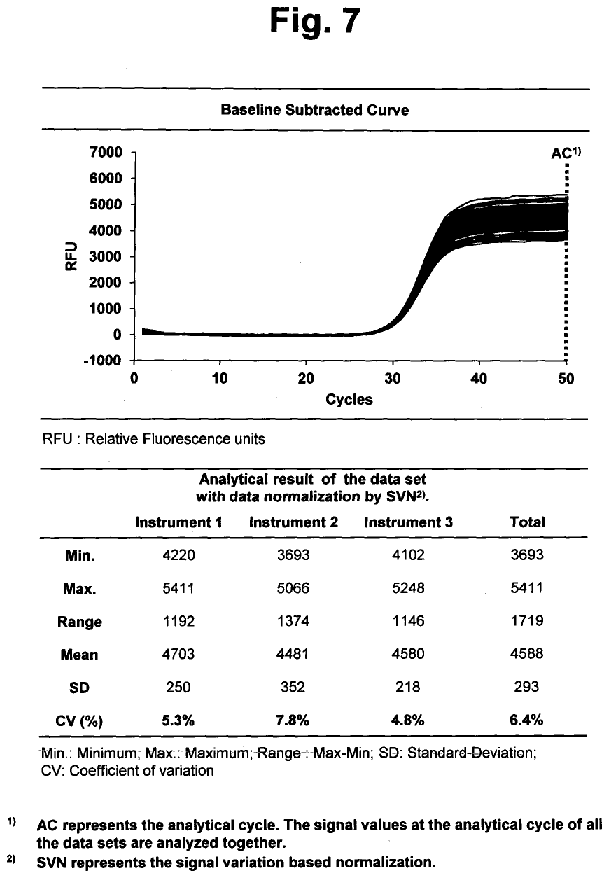

FIG. 7 shows the baseline subtracted amplification curves and the intra- and inter-instrument coefficients of variations for the calibrated data sets which were provided by calibrating the data sets obtained from the instrument with a hardware adjustment through the steps 1 to 3.

As shown in FIG. 7, the intra-instrument coefficients of variations of the amplification signals were 5.3%, 7.8% and 4.8%, respectively and the inter-instrument coefficient of variation of the amplification signal was 6.4%. It would be noticeable that the intra- and inter-instrument coefficients of variations of the amplification signals in this Example were the same as those in Example <1-4-1> represented by FIG. 5C but their signal intensities were different from each other.

Therefore, it would be understood that the SVN exhibits calibration effects on data sets by application of the TSC and moreover the R-TSC contributes to suitably adjusting signal intensities of calibrated data sets.

The method for calibrating signals from a real-time PCR instrument using the SVN can be utilized to reduce the inter-instrument variations of signals with convenient and software-wise approach. In addition, it would be appreciated that the calibration of signals of each well within an instrument by using TSC obtained from each well can reduce the intra-instrument variations of signals.

Furthermore, the calibration method of the SVN can be universally applied to various real-time PCR instruments because it calibrates data sets in a software-wise manner rather than hardware-wise. In addition to these, the SVN method of the invention is able to additionally calibrate signals that have been already hardware-wise calibrated. Instruments such as real-time PCR instruments have been generally subjected to a hardware adjustment before being put on a market. Where applied to instruments with hardware adjustment, the present method can offer instruments to provide more precisely calibrated signal values.

### Example 2: Calibration of Melting Data Set by Signal Variation-Based Normalization (SVN) and Analysis of Calibrated Data Set

In the above Example 1, the nucleic acid amplification data sets were calibrated using the SVN. In Example 2, it was investigated whether the melting data sets could be calibrated software-wise by the present method.

The signal variations in the following three groups of melting data sets were compared and analyzed: (i) a group of melting data sets obtained from an instrument without a hardware adjustment; (ii) a group of melting data sets obtained from an instrument with a hardware adjustment; and (iii) a group of calibrated melting data sets obtained by calibrating the melting data sets software-wise using the SVN.

<2-1> Preparation of Melting Data Sets

A melting analysis for a target nucleic acid molecule was performed using a PTOCE assay (WO 2012/096523) as a signal-generating means with 50 cycles of amplification on the six CFX96™ Real-Time PCR Detection Systems (Bio-Rad) listed in Table 5. The target nucleic acid molecule was a DNA of human beta-globin. The interactive dual label was provided by CTO labeled with a reporter molecule (Quasar 670) and a quencher molecule (BHQ-2) (dual-labeled CTO).

The reaction was conducted in the tube containing a target nucleic acid molecule, a downstream primer, an upstream primer, dual-labeled CTO, PTO and Master Mix containing MgCl₂, dNTPs and Taq DNA polymerase. The tube containing the reaction mixture was placed on the real-time thermocycler (CFX96, Bio-Rad). The reaction mixture was denatured for 15 min at 95° C. and subjected to 50 cycles of 30 sec at 95° C., 60 sec at 60° C., 30 sec at 72° C. The melting data sets were obtained by detecting temperature-dependent fluorescent signals while the real-time PCR products were heated from 55° C. to 85° C. by 0.5° C. In melting data sets, temperatures are considered as cycles. A signal measurement unit may be either time in signal amplification data sets or temperature in melting data sets.

Twenty-four (24) reactions for each instrument were carried out under the same condition using samples containing the same target nucleic acid of the same concentration. Further, one reaction was also carried out simultaneously with the 24-reaction on each instrument using a sample containing a target analyte of known concentration, which was utilized as a standard reaction. By analyzing the melting data sets and standard melting data set obtained from the above reactions, the level of the intra- and inter-instrument melting signal variations and the level of reduction in melting signal variations by the present method were analyzed.

A total six groups of raw melting data sets consisting of fluorescence values (RFUs) for temperatures were prepared by using a total six PCR instruments (three non hardware-adjusted instruments and three hardware-adjusted instruments). Each group includes 24-data sets and 1-standard data set obtained from 96-well reactions.

The derivatives of the raw melting data sets were calculated as the negative derivatives from the raw melting data sets. In order to calculate the derivatives, the Least Square Method was applied to the raw melting data sets according to the following equation and the derivatives of the raw melting data sets were obtained as the negative derivatives.

$${s}_{i} = - \frac{{\sum }_{i = I - a}^{I + b} ( {x}_{i} - \overline{x} ) ( {y}_{i} - \overline{y} )}{{\sum }_{i = I - a}^{I + b} {( {x}_{i} - \overline{x} )}^{2}}$$

$$\overline{x} = \frac{{\sum }_{i = I - a}^{I + b} {x}_{i}}{n} , \overline{y} = \frac{{\sum }_{i = I - a}^{I + b} {y}_{i}}{n}$$

I: a cycle number of data sets of which derivatives are to be calculated

xᵢ: a cycle number of i^{th} cycle

yᵢ: a signal intensity measured at i^{th} cycle

sᵢ the amount of data change at i^{th} cycle

“a” and “b”: an integer from 0 to 10

n: a+b+1, a number of data used to calculate derivatives

x̅: a mean value of cycle numbers from “I-a” to “I+b”

y̅: a mean value of signal intensities measured at cycles from “I-a” to “I+b”

In Examples, “1” is used for “a” and “b”. For data points at which “I-a” is less than “1”, “a” may be altered to permit “I-a” to become “1”. For data points at which “I+b” is more than the number of all data points, “b” may be altered to permit “I+b” to be equal to the number of all data points.

The melting curves were obtained by plotting the raw melting data sets and the melting derivative curves (melting peaks) were obtained by plotting the derivatives of the raw melting data sets.

TABLE 5

| Hardware Adjustment | Instrument Number | Real-time PCR Instrument |
|---|---|---|
| No adjustment | Instrument 1 | CFX96 Real-time Cycler |
| | Instrument 2 | (Bio-Rad) |
| | Instrument 3 | |
| Adjustment | Instrument 1 | |
| | Instrument 2 | |
| | Instrument 3 | |

<2-2> Analysis of Melting Data Set Obtained from Instrument without Hardware Adjustment

The raw melting data sets and their derivatives obtained in Example <2-1> were used. The signal variations were analyzed for three groups of raw melting data sets obtained from the instruments without a hardware adjustment and for three groups of the derivatives of the raw melting data sets.

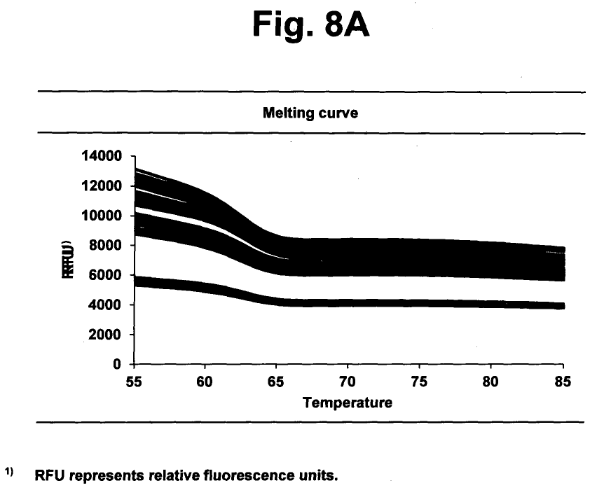

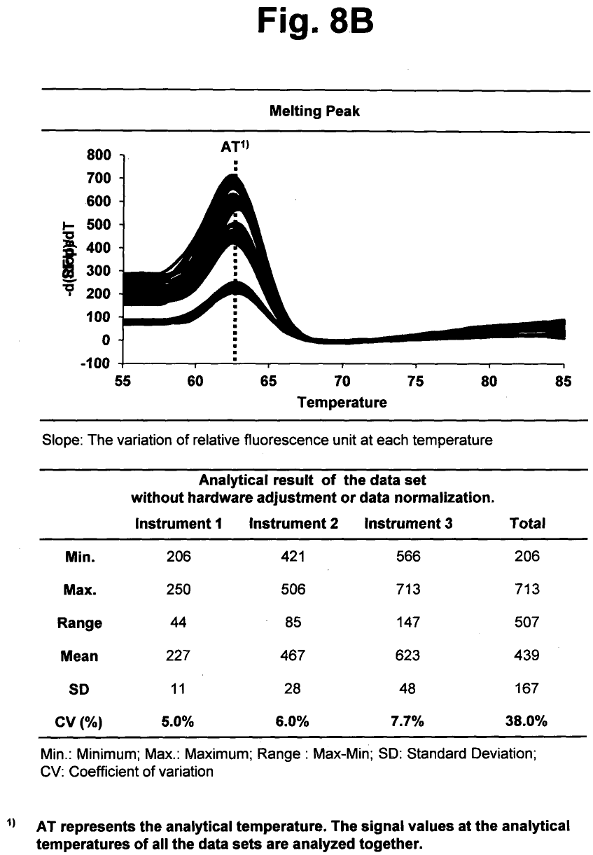

The melting curves were obtained by plotting the raw melting data sets in order to identify the overall melting signal patterns of three instruments (FIG. 8A).

As a result of analyzing the melting curves shown in FIG. 8A, the signals between the instruments were divided from each other, which is unlike to a theoretical expectation that the same value of derivatives will be plotted for melting reactions under the same condition.

In order to compare signal variations in the melting curve analysis, the melting derivative curves (melting peaks) were prepared by plotting the derivatives of the raw melting data sets which were obtained from the respective three instruments. The variation of the melting peak is the variation of the derivatives of the melting data set.

The point at which the value (slope) of the melting peak was maximized was designated as an analytical temperature and the coefficients of variation of the value of the melting peak at the analytical temperature were calculated.

The coefficient of variation of the melting peak i.e., the coefficient of variation of derivative of the melting data set is the coefficient of variation of the value of melting peaks at the analytical temperature. The coefficient of variation was calculated as described in Example <1-2>.

The coefficient of variation of the melting peak was represented in FIG. 8B. The intra-instrument coefficients of variations of the melting peaks were analyzed as 5.0%, 6.0%, and 7.7%, respectively and the inter-instrument coefficient of variation of the melting peaks was analyzed as 38.0%.

<2-3> Analysis of Melting Data Set Obtained from Instrument with Hardware Adjustment

The signal variations were analyzed by the same method as described in Example <2-2> between three groups of the raw melting data sets obtained from the instrument with a hardware adjustment in Example <2-1> and three groups of the derivatives of the raw melting data sets.

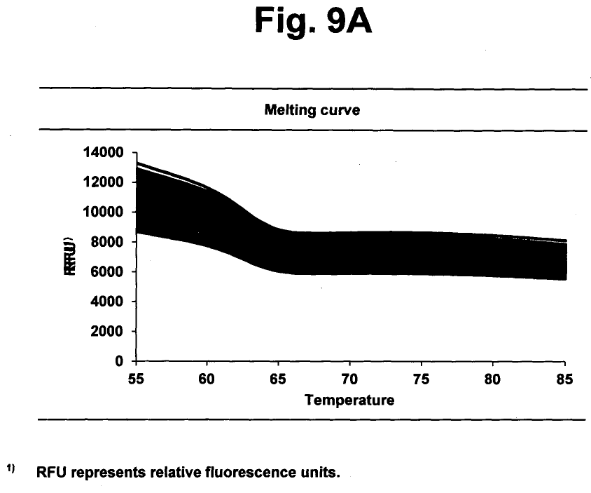

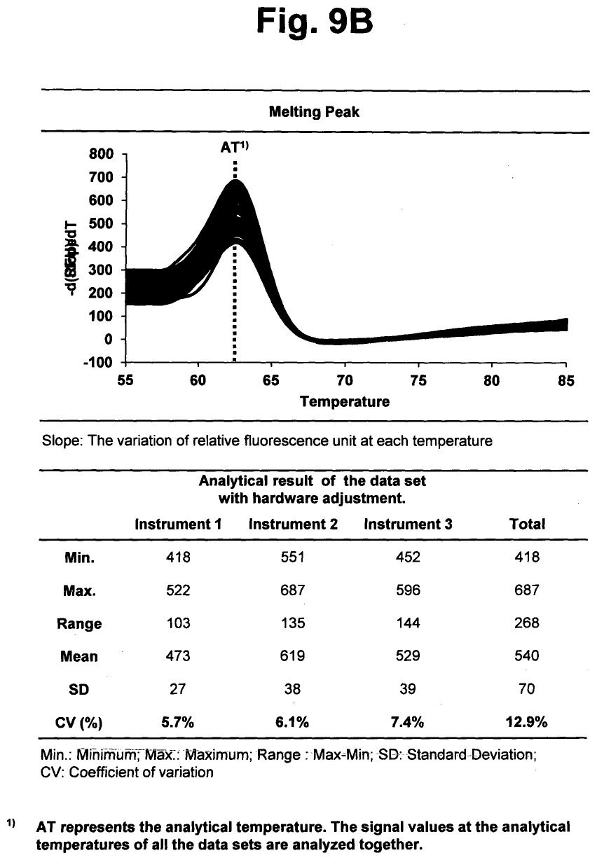

The melting curves were analyzed according to the same method as described in Example <2-2>. As shown in FIG. 9A, it was revealed that the inter-instrument signal variations were reduced compared to the melting data sets obtained from the instrument without a hardware adjustment.

The coefficient of variation of the melting derivative curves (melting peaks) was calculated according to the same method as described in Example <2-2>. As shown in FIG. 9B, it was revealed that the intra-instrument coefficients of variations of the melting peaks analyzed as 5.7%, 6.1%, and 7.4%, respectively and the inter-instrument coefficient of variation of the melting peaks was analyzed as 12.9%.

When the above results were compared with the results of the melting data sets obtained from the instrument without a hardware adjustment in Example <2-2>, it was proved that the inter-instrument coefficient of variation of the melting peaks was reduced by 25.1% P (percentage points) while there was negligent difference in the intra-instrument coefficient of variation of the melting peak.

From the above results, it can be concluded that even though the calibration by the hardware adjustment can reduce partly the inter-instrument coefficient of variation of the melting peak, a considerable level of signal variations between the instruments still exists.

<2-4> Analysis of Melting Data Set Software-Wise Calibrated Using SVN

<2-4-1> Calibration of Melting Data Set by SVN Using Total Signal Change Value

In this Example, the melting data sets were calibrated by the SVN applying the total signal change value (TSC) to the melting data sets obtained from each instrument. The raw melting data sets of six groups obtained in Example <2-1> were software-wise calibrated using the SVN according to the following steps:

<Step 1>

An instrument-specific standard melting data set was obtained by performing a standard signal-generating process using a target analyte of standard concentration under the same reaction condition as that of signal-generating processes performed for obtaining melting data sets from an experimental sample.

The total signal change value (TSC) was calculated from the melting peak of the standard melting data set. The slope at the maximum value of the melting peak from the standard melting data set was designated as the total signal change value (TSC).

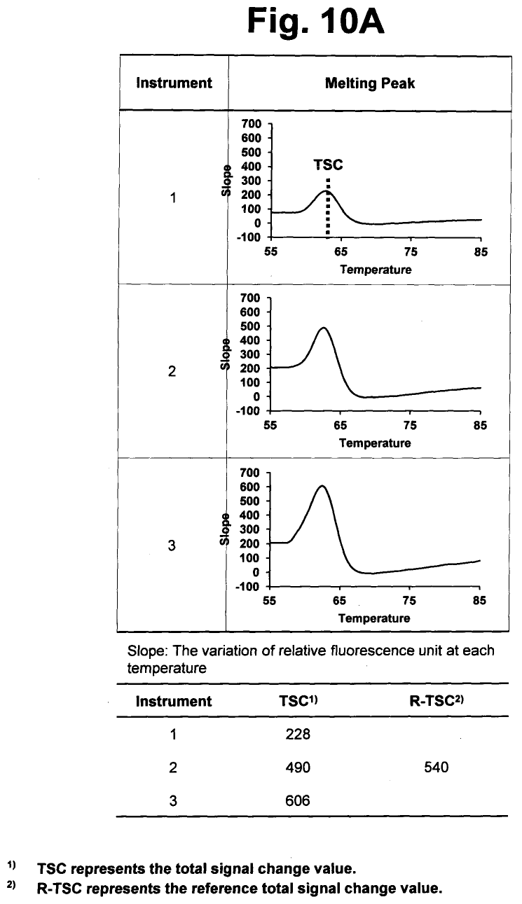

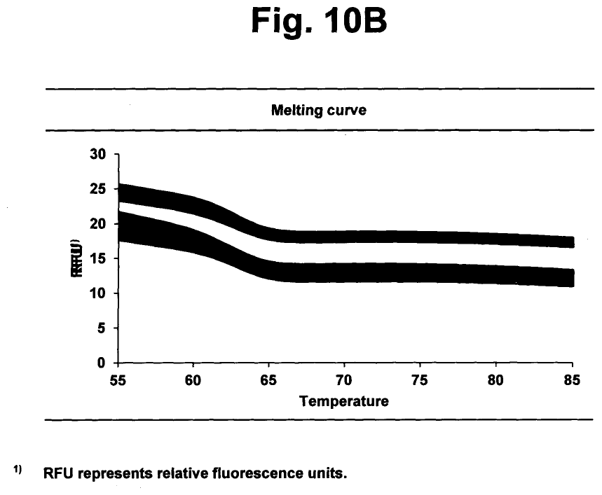

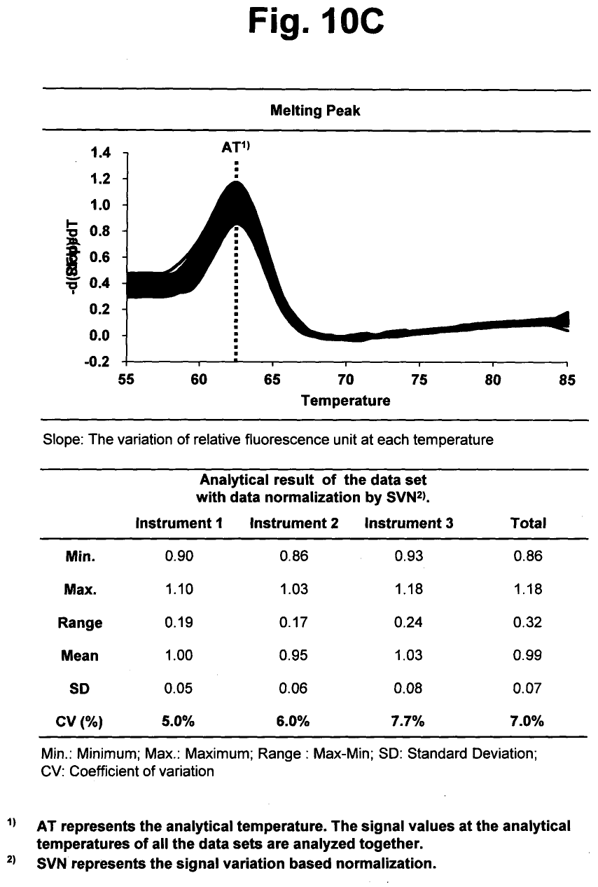

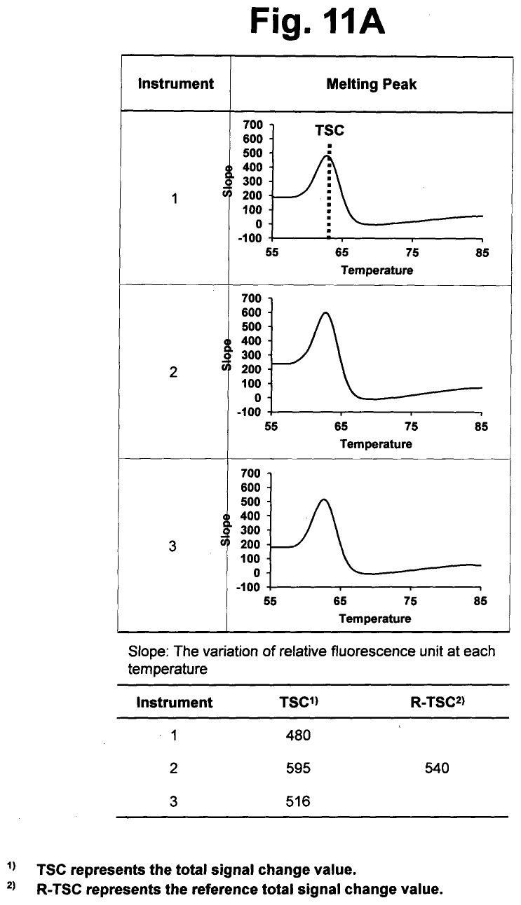

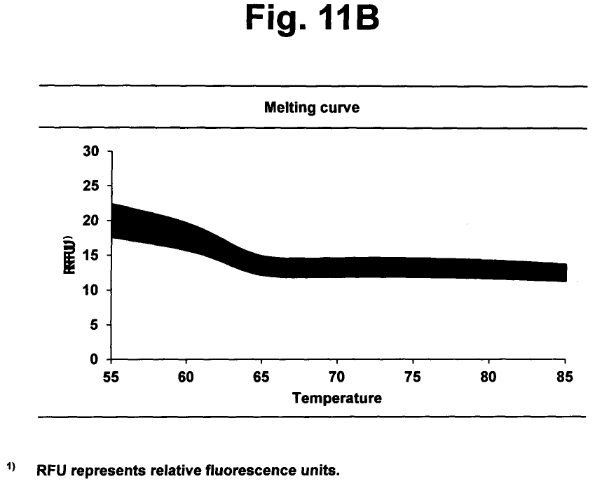

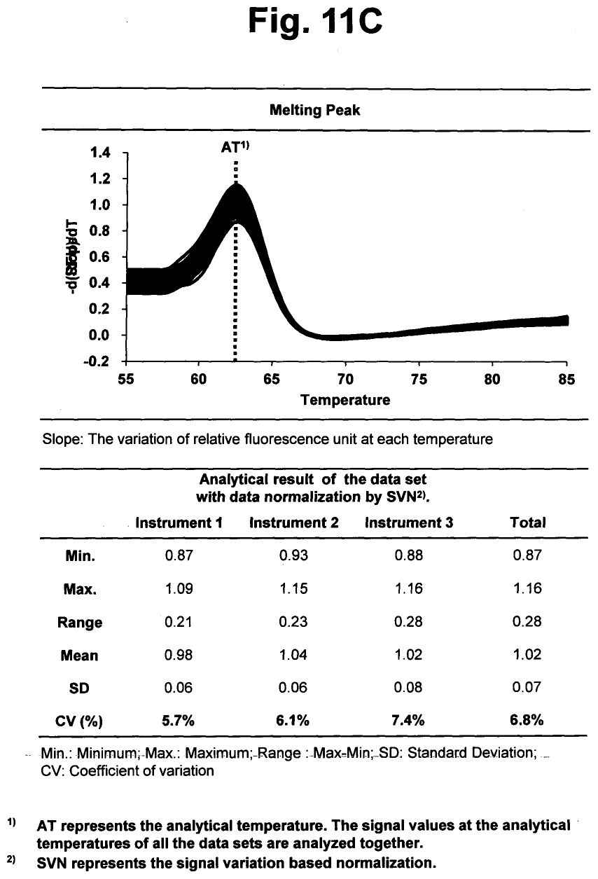

In this Example, a standard melting data set was prepared from each instrument and total signal change values each was calculated. The total signal change values (TSCs) of the respective standard melting data sets obtained from the instruments 1, 2, and 3 without or with a hardware adjustment were measured as shown in Table 6 (FIGS. 10A and 11A).

TABLE 6

| | | TSC of Standard Melting |
|---|---|---|
| Hardware Adjustment | Instrument Number | Data Sets (Slope) |
| − | Instrument 1 | 228 |
| − | Instrument 2 | 490 |
| − | Instrument 3 | 606 |
| + | Instrument 1 | 480 |
| + | Instrument 2 | 595 |
| + | Instrument 3 | 516 |

<Step 2>

The signal values at all temperatures of the raw melting data sets obtained from each instrument were calibrated by using the instrument-specific TSCs obtained from each instrument.

Calibrated Signal Value (RFU)=Signal Value of Raw Melting Data Set (RFU)÷TSC

The calibrated six groups of melting data sets were obtained by calibrating the six groups of the raw melting data sets provided in Example <2-1> according to the above steps 1 to 2.

A. Analysis of the Results of Calibration of Melting Data Sets Obtained from an Instrument without a Hardware Adjustment

The instrument-specific TSCs were calculated from the respective standard melting data sets obtained from the instruments 1, 2, and 3 without a hardware adjustment (FIG. 10A). The melting data sets obtained from the instruments 1, 2, and 3 without a hardware adjustment were calibrated by the SVN using the instrument-specific TSCs, and the resulting calibrated melting data sets were analyzed.

FIG. 10B shows the melting curves representing the results of calibrating the melting data sets obtained from the respective instruments without a hardware adjustment through the above steps 1 to 2. FIG. 10C shows the intra- and inter-instrument coefficients of variations obtained from the melting peaks for the results of calibrating the melting data sets obtained from the instrument without a hardware adjustment through the above steps 1 to 2. The melting curves were obtained by plotting the melting data sets calibrated by the SVN with the TSCs, in which the raw melting data sets had been obtained from the instrument without a hardware adjustment.

FIG. 10B shows the melting curves obtained by plotting the calibrated melting data sets, in which the intensities of the melting signals can be compared. The signal values of the melting data sets from the three instruments were calibrated such that the melting signals became similar to one another.

The derivatives of the raw melting data sets were obtained from the calibrated melting data sets, the melting derivative curves (melting peaks) were obtained by plotting the derivative of the raw melting data sets, and the coefficient of variation was calculated from the melting derivative curves.

The coefficients of variations of the melting peaks (i.e., the coefficients of variations of derivatives of the melting data sets) were analyzed. The intra-instrument coefficients of variations of the melting peaks were 5.0%, 6.0%, and 7.7%, respectively and the inter-instrument coefficient of variation of the melting peaks was 7.0%.

The following three coefficients of variations were compared and analyzed: (i) the coefficient of variation of the melting peaks calculated from the derivatives of the melting data sets obtained from the instrument without a hardware adjustment in Example <2-2>; (ii) the coefficient of variation of the melting peaks calculated from the derivatives of the melting data sets obtained from the instrument with a hardware adjustment in Example <2-3>; and (iii) the coefficient of variation of the melting peaks of the derivatives of the calibrated melting data sets provided by calibrating the melting data sets by the SVN using the TSCs, wherein the melting data sets had been obtained from the instrument with a hardware adjustment.

As shown in Table 7, the calibrated melting data sets by the SVN using the TSCs have following characteristics: When compared with the melting data sets obtained from the instrument without a hardware adjustment, the inter-instrument coefficient of variation of the melting peak was remarkably reduced by 31.0% P (percentage points). In addition, when compared with the melting data sets obtained from the instrument with a hardware adjustment, the inter-instrument coefficient of variation of the derivative was significantly reduced by 5.9% P (percentage points).

It would be demonstrated that the signal calibration method of SVN using the total signal change values (TSCs) can effectively reduce inter-instrument signal variations of melting data sets, addressing that a melting signal calibration effect being more remarkable than that of a conventional hardware adjustment can be successfully accomplished by only the SVN using the signal total change value without a hardware adjustment of an instrument.

TABLE 7

| Calibration | SVN Using the TSCs | − | − | + |
|---|---|---|---|---|
| Method | Hardware Adjustment | − | + | − |
| Result of | Instrument 1 | 5.0 | 5.7 | 5.0 |
| Analysis | | | | |
| of Melting | | | | |
| Signal | | | | |
| (Coefficient | | | | |
| of Variation, CV %) | | | | |
| | Instrument 2 | 6.0 | 6.1 | 6.0 |
| | Instrument 3 | 7.7 | 7.4 | 7.7 |
| | Total | 38.0 | 12.9 | 7.0 |

B. Analysis of the Results of Calibration of Data Sets Obtained from an Instrument with a Hardware Adjustment

The instrument-specific TSCs were calculated from the respective standard melting data sets obtained from the instruments 1, 2, and 3 with a hardware adjustment (FIG. 11A). The melting data sets obtained from the instruments 1, 2, and 3 with a hardware adjustment were calibrated by the SVN using the instrument-specific TSCs, and the resulting calibrated melting data sets were analyzed.

FIG. 11B shows the melting curves representing results of calibrating the melting data sets obtained from the respective instruments with a hardware adjustment through the above steps 1 to 2. FIG. 11C shows the intra- and inter-instrument coefficients of variations obtained from the melting peaks representing results of calibrating the melting data sets obtained from the instrument with a hardware adjustment through the above steps 1 to 2. The melting curves were obtained by plotting the calibrated melting data sets by the SVN with the TSCs, in which the raw melting data sets had been obtained from the instrument with a hardware adjustment.

FIG. 11B shows the melting curves of the calibrated melting data sets in which the intensities of the melting signals can be compared. The signal values of the melting data sets from the three instruments were calibrated such that the melting signals became similar to one another.

The coefficients of variations of the melting peaks were analyzed (FIG. 11C). The intra-instrument coefficients of variations of the melting peaks were 5.7%, 6.1%, and 7.4%, respectively and the inter-instrument coefficient of variation of the melting peaks was 6.8%.

The following three coefficients of variations were compared and analyzed: (i) the coefficient of variation of the melting peaks calculated from the derivatives of the melting data sets obtained from the instrument without a hardware adjustment in Example <2-2>; (ii) the coefficient of variation of the melting peaks calculated from the derivatives of the melting data sets obtained from the instrument with a hardware adjustment in Example <2-3>; and (iii) the coefficient of variation of the melting peaks of the derivatives of the calibrated melting data sets provided by calibrating the melting data sets by the SVN using the TSCs, wherein the melting data sets had been obtained from the instrument with a hardware adjustment.

As shown in Table 8, the calibrated melting data sets by the SVN using the TSCs have following characteristics: When compared with the melting data sets obtained from the instrument without a hardware adjustment, the inter-instrument coefficient of variation of the melting peak was remarkably reduced by 31.2% P (percentage points). In addition, when compared with the melting data sets obtained from the instrument with a hardware adjustment, the inter-instrument coefficient of variation of the derivative was significantly reduced by 6.1% P (percentage points).

It would be understood that melting data sets obtained from the instrument with a hardware adjustment can be further calibrated by the SVN, such that an inter-instrument variation in melting data sets can be more precisely corrected.

TABLE 8

| Calibration | SVN Using the TSCs | − | − | + |
|---|---|---|---|---|
| Method | Hardware Adjustment | − | + | + |
| Result of Analysis | Instrument 1 | 5.0 | 5.7 | 5.7 |
| of Melting Signal | Instrument 2 | 6.0 | 6.1 | 6.1 |
| (Coefficient | Instrument 3 | 7.7 | 7.4 | 7.4 |
| of Variation, CV %) | Total | 38.0 | 12.9 | 6.8 |

<2-4-2> Calibration of Melting Data Set by SVN Using Total Signal Change Value and Reference Total Signal Change Value

In this Example, the melting data sets were calibrated by the SVN applying the total signal change value (TSC) and R-TSC to the melting data sets obtained from each instrument. The raw melting data sets of six groups obtained in Example <2-1> were software-wise calibrated using the SVN according to the following steps:

<Step 1>

As shown in Example <2-4-1>, the total signal change values (TSCs) were measured from the respective standard melting data sets (Table 6).

<Step 2>

The signal values at all temperatures of the raw melting data sets obtained from each instrument were calibrated respectively using the instrument-specific TSC obtained from each instrument.

1^{st} Calibrated Signal Value (RFU)=Signal Value of Raw Melting Data Set (RFU)÷TSC

<Step 3>

In this Example, a slope value of 540 was designated as the R-TSC (FIGS. 10A and 11A), which is similar to the mean of the total signal change values of the data sets obtained from three instruments with a hardware adjustment of Example <2-1> (FIG. 9B). The 2^{nd} calibrated signal values were obtained by applying the R-TSC to the 1^{st} calibrated signal values.

2^{nd} Calibrated Signal Value (RFU)=1^{st} Calibrated Signal Value (RFU)×R-TSC

The calibrated six groups of melting data sets were obtained by calibrating the six groups of the raw melting data sets provided in Example <2-1> according to the above steps 1 to 3.

A. Analysis of the Results of Calibration of Melting Data Sets Obtained from an Instrument without a Hardware Adjustment

The melting data sets obtained from an instrument without a hardware adjustment were calibrated by the SVN using the TSC and R-TSC, and the resulting calibrated melting data sets were analyzed. As a result, it was found that the signal variations in the calibrated melting data sets were the same as those represented in FIG. 10B but the signal intensities (Y-axis values) were different from those represented in FIG. 10B.

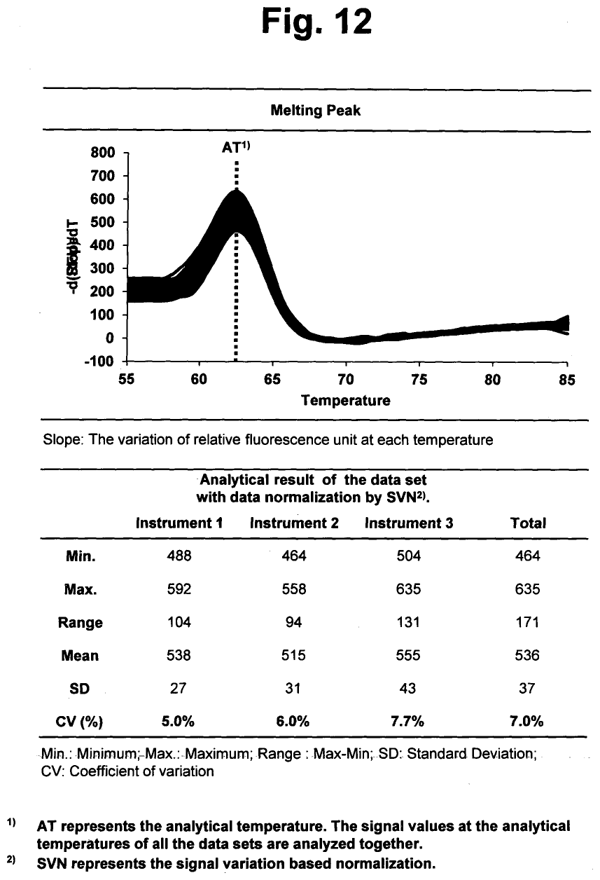

FIG. 12 shows the melting peaks of the calibrated melting data sets obtained by plotting the derivatives of the calibrated melting data sets and the intra- and inter-instrument coefficients of variations obtained from the melting peaks for the calibrated melting data sets obtained from the instrument without a hardware adjustment through the above steps 1 to 3.

As shown in FIG. 12, the intra-instrument coefficients of variations of the melting peaks were 5.0%, 6.0% and 7.7%, respectively and the inter-instrument coefficient of variation of the melting peaks was 7.0%. It would be noticeable that the intra- and inter-instrument coefficients of variations of the melting peaks of the normalized data sets of this Example were the same as those of Example <2-4-1> represented by FIG. 10C but their signal intensities (Y-axis values) were different from each other.

B. Analysis of the Results of Calibration of Melting Data Sets Obtained from an Instrument with a Hardware Adjustment

The melting data sets obtained from an instrument with a hardware adjustment were calibrated by the SVN using both the TSC and R-TSC, and the resulting calibrated melting data sets were analyzed. As a result, it was found that the signal variations in the calibrated melting data sets were the same as those represented in FIG. 11B but the signal intensities (Y-axis values) were different from those represented in FIG. 11B.

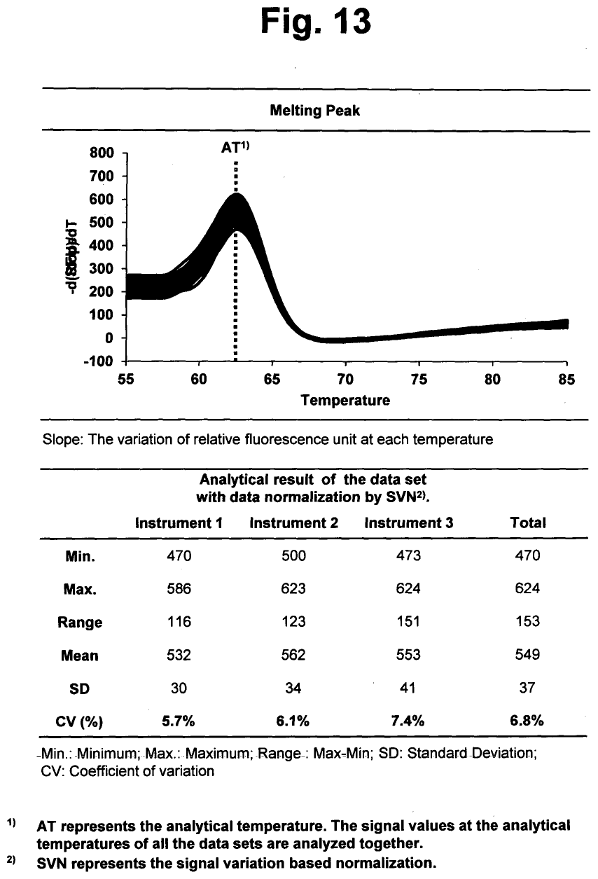

FIG. 13 shows the melting peaks of the calibrated melting data sets obtained by plotting the derivatives of the calibrated melting data sets and the intra- and inter-instrument coefficients of variations obtained from the melting peaks for the calibrated melting data sets obtained from the instrument with a hardware adjustment through the above steps 1 to 3.

As shown in FIG. 13, the intra-instrument coefficients of variations of the melting peaks were 5.7%, 6.1% and 7.4%, respectively and the inter-instrument coefficient of variation of the melt peaks was 6.8%. It would be noticeable that the intra- and inter-instrument coefficients of variations of the melting peaks of the normalized data sets of this Example were the same as those of Example <2-4-1> represented by FIG. 11C but their signal intensities (Y-axis values) were different from each other.

Therefore, it would be understood that the SVN exhibits calibration effects on melting data sets by application of the TSC and moreover the R-TSC contributes to suitably adjusting signal intensities of calibrated data sets.

Accordingly, the method of a signal calibration using the SVN is also applicable to calibration of melting signals as well as amplification signals with similar effects. Because the melting curve analysis requires fine control of temperatures for detection of signals, there is a higher possibility of the inter-instrument signal variations in the melting curve analysis than the amplification curve analysis. Therefore, it would be appreciated that advantages of the present calibration method will be highlighted in the melting curve analysis.

### Example 3: Calibration of Data Set by Signal Variation-Based Normalization (SVN) with Calibration Coefficient and Analysis of Calibrated Data Set

In this Example, the SVN using a calibration coefficient was used for calibrating variations in amplifying and melting signals of data sets.

The signal variations in the following three groups of data sets were compared and analyzed: (i) a group of data sets obtained from an instrument without a hardware adjustment; (ii) a group of data sets obtained from an instrument with a hardware adjustment; and (iii) a group of data sets software-wise calibrated by the SVN.

<3-1> Calibration and Analysis of the Amplification Data Set

<3-1-1> Preparation of Amplification Data Set

The raw amplification data sets obtained in Example <1-1> were used.

<3-1-2> Analysis of Amplification Data Set Obtained from Instrument without Hardware Adjustment

As in the above Example <1-2>, the intra-instrument coefficients of variations of the amplification signals of the instruments 1, 2, and 3 were analyzed as 5.2%, 9.1%, and 4.5%, respectively and the inter-instrument coefficient of variation of the amplification signals of the instruments 1, 2, and 3 was analyzed as 49.3% (FIG. 2B).

<3-1-3> Analysis of Amplification Data Set Obtained from Instrument with Hardware Adjustment

As in the above Example <1-3>, the intra-instrument coefficients of variations of the amplification signals of the instruments 1, 2, and 3 were analyzed as 5.3%, 7.8%, and 4.8%, respectively and the inter-instrument coefficient of variation of the amplification signals of the instruments 1, 2, and 3 was analyzed as 17.7% (FIG. 3B).

<3-1-4> Analysis of Amplification Data Set Software-Wise Calibrated Using SVN

In this Example, the amplification data sets were calibrated by the SVN applying the calibration coefficients to data sets obtained from each instrument. The raw amplification data sets of six groups obtained in Example <1-1> were software-wise calibrated using the SVN according to the following steps:

<Step 1>

As in the above Example <1-4-1>, the total signal change values (TSCs) of the standard amplification data sets obtained from the instruments 1, 2, and 3 without or with a hardware adjustment were measured (Table 2).

<Step 2>

As in the above Example <1-4-2>, the value of RFU 4500 was designated as the R-TSC.

<Step 3>

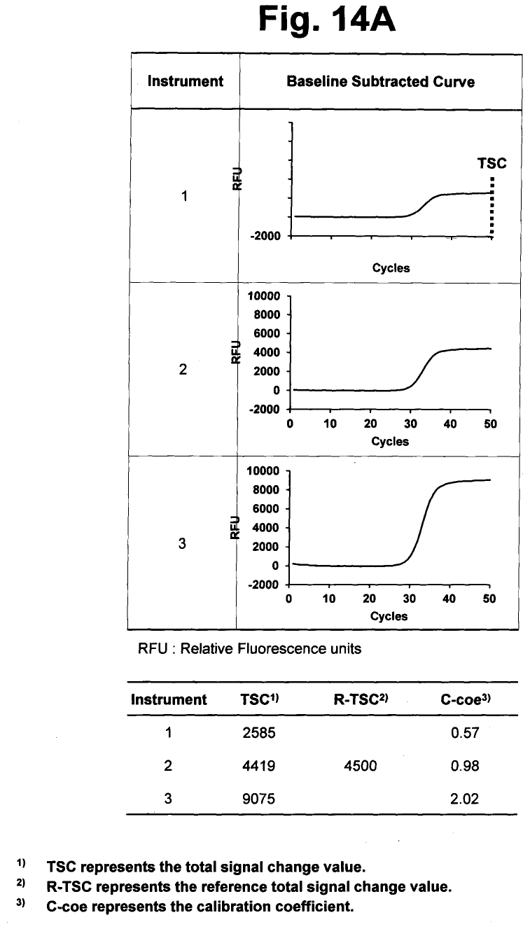

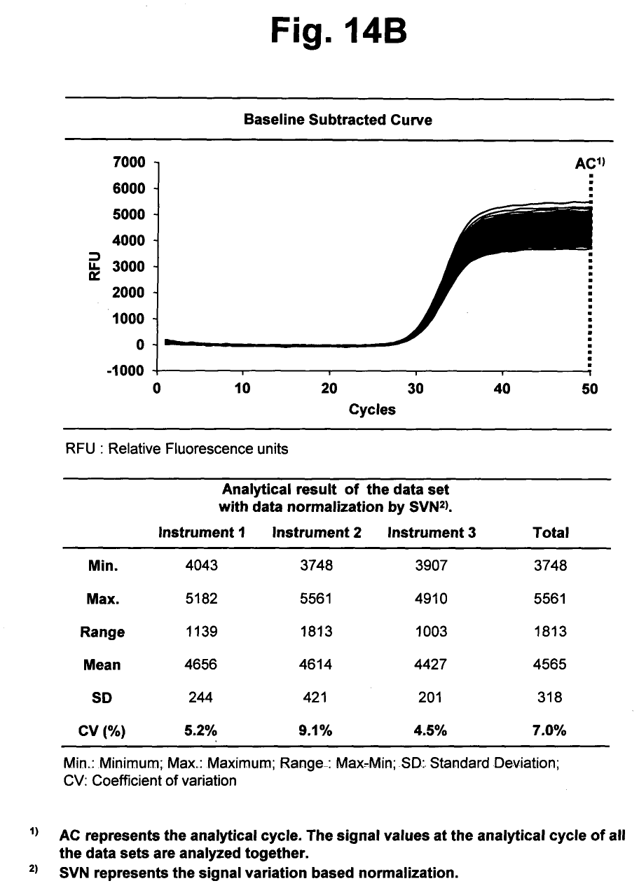

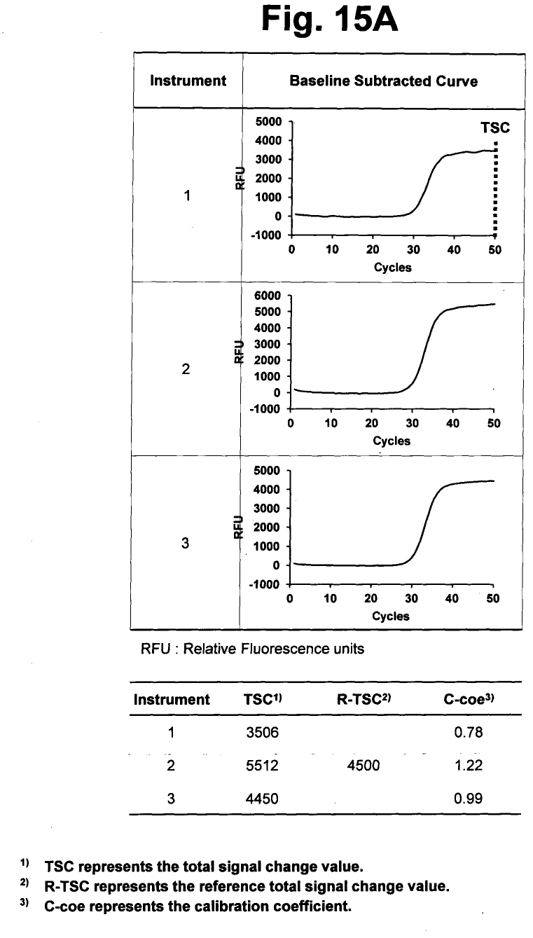

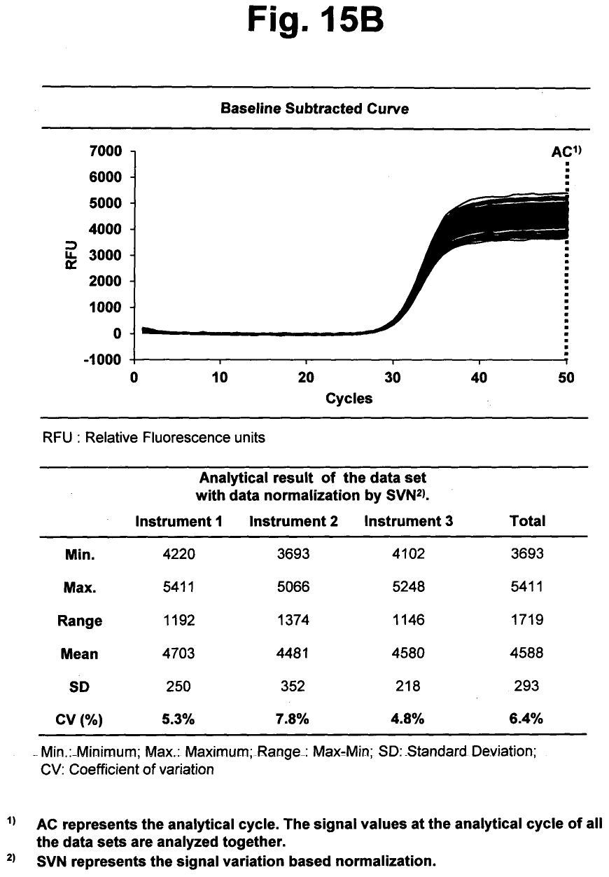

As shown in Table 9, a calibration coefficient for each instrument was calculated from both the TSC of each instrument and the R-TSC as follows (FIGS. 14A and 15A):

Calibration Coefficient=TSC÷R-TSC

TABLE 9

| | | A) | | |
|---|---|---|---|---|
| | | Total Signal | B) | |
| | | Change Value | Reference | C) |
| Hardware | | of Standard | Total Signal | Ratio of |
| Adjust- | Instrument | Data Set | Change Value | TSC to R-TSC |
| ment | Number | (TSC) | (R-TSC) | [A/B] |
| − | 1 | 2585 | 4500 | 0.57 |
| | 2 | 4419 | 4500 | 0.98 |
| | 3 | 9075 | 4500 | 2.02 |
| + | 1 | 3506 | 4500 | 0.78 |
| | 2 | 5512 | 4500 | 1.22 |
| | 3 | 4450 | 4500 | 0.99 |

<Step 4>

The signal values at all cycles of the data sets obtained from each instrument were calibrated using the calibration coefficient obtained from each instrument.

Calibrated Signal Value (RFU)=Signal Value of Raw Amplification Data Set (RFU)÷Calibration coefficient

The calibrated six groups of data sets were obtained by calibrating the six groups of the raw data sets provided in Example <1-1> according to the above steps 1 to 4.

A. Analysis of the Results of Calibration of Amplification Data Sets Obtained from an Instrument without a Hardware Adjustment

The data sets obtained from an instrument without a hardware adjustment were calibrated by the SVN using a calibration coefficient, and the resulting calibrated data sets were analyzed (FIG. 14B).

As shown in FIG. 14B, the intra-instrument coefficients of variations of the amplification signals were 5.2%, 9.1% and 4.5%, respectively and the inter-instrument coefficient of variation of the amplification signal was 7.0%.

B. Analysis of the Results of Calibration of Amplification Data Sets Obtained from an Instrument with a Hardware Adjustment

The data sets obtained from an instrument with a hardware adjustment were calibrated by the SVN using a calibration coefficient, and the resulting calibrated data sets were analyzed (FIG. 15B).

As shown in FIG. 15B, the intra-instrument coefficients of variations of the amplification signals were 5.3%, 7.8% and 4.8%, respectively and the inter-instrument coefficient of variation of the amplification signal was 6.4%.

As a result of analysis, it was demonstrated that the results of calibration by the SVN using calibration coefficient (C-coe) were the same as the results of the calibration by the SVN using the TSC and R-TSC in the above example <1-4-2>.

<3-2> Calibration and Analysis of the Melting Data Set

<3-2-1> Preparation of Melting Data Set

The raw melting data sets obtained in Example <2-1> were used.

<3-2-2> Analysis of Melting Data Set Obtained from Instrument without Hardware Adjustment

As in the above Example <2-2>, the intra-instrument coefficients of variations of the melting signals of the instruments 1, 2, and 3 were analyzed as 5.0%, 6.0%, and 7.7%, respectively and the inter-instrument coefficient of variation of the melting signals of the instruments 1, 2, and 3 was analyzed as 38.0% (FIG. 8B).

<3-2-3> Analysis of Melting Data Set Obtained from Instrument with Hardware Adjustment

As in the above Example <2-3>, the intra-instrument coefficients of variations of the melting signals of the instruments 1, 2, and 3 were analyzed as 5.7%, 6.1%, and 7.4%, respectively and the inter-instrument coefficient of variation of the melting signals of the instruments 1, 2, and 3 was analyzed as 12.9% (FIG. 9B).

<3-2-4> Analysis of Melting Data Set Software-Wise Calibrated Using SVN

In this Example, the melting data sets were calibrated by the SVN applying the calibration coefficients to melting data sets obtained from each instrument. The raw melting data sets of six groups obtained in Example <2-1> were software-wise calibrated using the SVN according to the following steps:

<Step 1>

As in the above Example <2-4-1>, the total signal change values (TSCs) of the standard melting data sets obtained from the instruments 1, 2, and 3 without or with a hardware adjustment were measured (Table 6).

<Step 2>

As in the above Example <2-4-2>, the value of slope 540 was designated as the R-TSC.

<Step 3>

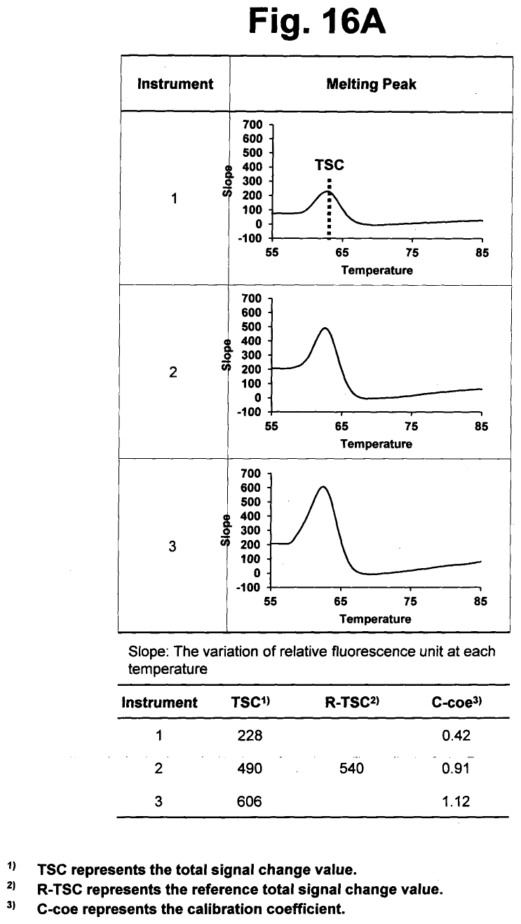

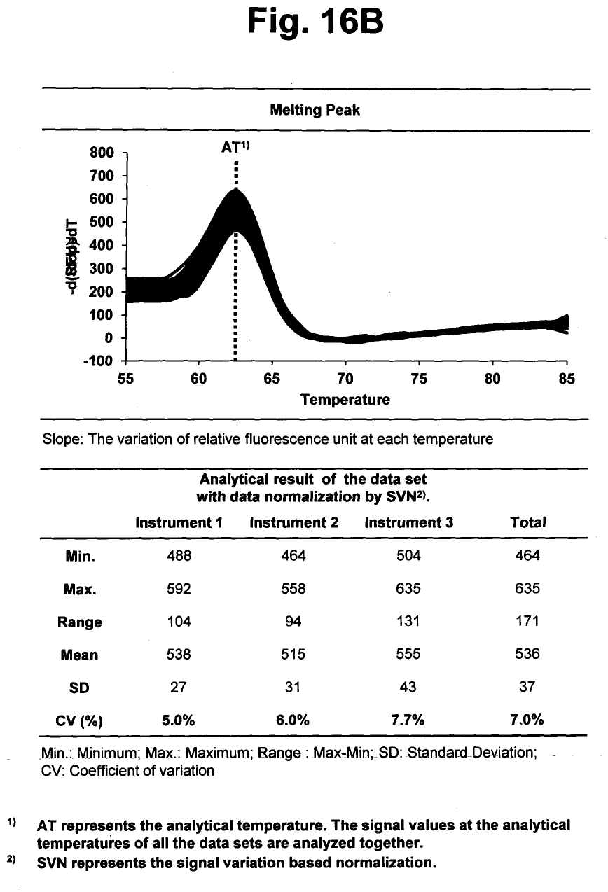

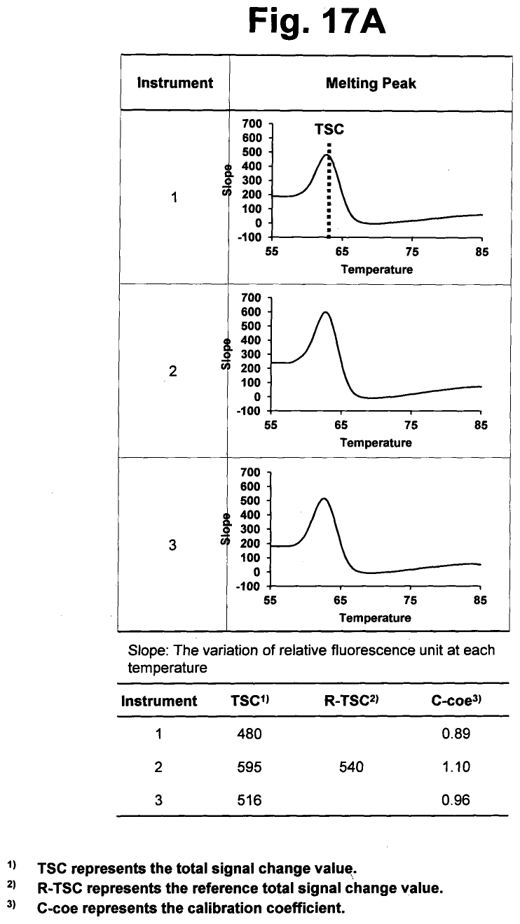

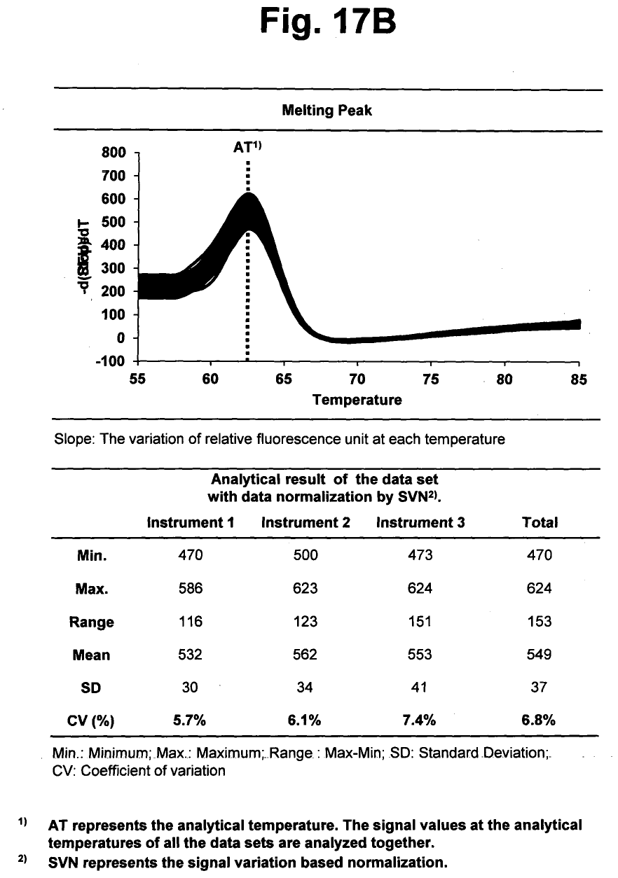

As shown in Table 10, a calibration coefficient was calculated from the TSC and R-TSC as follows (FIGS. 16A and 17A).

Calibration Coefficient=TSC÷R-TSC

TABLE 10

| | | A) | | |
|---|---|---|---|---|
| | | Total Signal | B) | |
| | | Change Value | Reference | C) |
| Hardware | | of Standard | Total Signal | Ratio of |
| Adjust- | Instrument | Data Set | Change Value | TSC to R-TSC |
| ment | Number | (TSC) | (R-TSC) | [A/B] |
| − | 1 | 228 | 540 | 0.42 |
| | 2 | 490 | 540 | 0.91 |
| | 3 | 606 | 540 | 1.12 |
| + | 1 | 480 | 540 | 0.89 |
| | 2 | 595 | 540 | 1.10 |
| | 3 | 516 | 540 | 0.96 |

<Step 4>

The signal values at all temperatures of the raw melting data sets obtained from each instrument of were calibrated by using the calibration coefficient calculated for each instrument.

Calibrated Signal Value (RFU)=Signal Value of Raw Melting Data Set (RFU)÷Calibration Coefficient

The calibrated six groups of melting data sets were obtained by calibrating the six groups of the raw melting data sets provided in Example <2-1> according to the above steps 1 to 4.

A. Analysis of the Results of Calibration of Melting Data Sets Obtained from an Instrument without a Hardware Adjustment

The melting data sets obtained from an instrument without a hardware adjustment were calibrated by the SVN using a calibration coefficient, and the resulting calibrated melting data sets were analyzed (FIG. 16B).

As shown in FIG. 16B, the intra-instrument coefficients of variations of the melting signals were 5.0%, 6.0% and 7.7%, respectively and the inter-instrument coefficient of variation of the melting signal was 7.0%.

B. Analysis of the Results of Calibration of Melting Data Sets Obtained from an Instrument with a Hardware Adjustment

The melting data sets obtained from an instrument with a hardware adjustment were calibrated by the SVN using a calibration coefficient, and the resulting calibrated data sets were analyzed (FIG. 17B).

As shown in FIG. 17B, the intra-instrument coefficients of variations of the melting signals were 5.7%, 6.1% and 7.4%, respectively and the inter-instrument coefficient of variation of the melting signal was 6.8%.

As a result of analysis, it was demonstrated that the results of calibration by the SVN using calibration coefficient (C-coe) were the same as the results of the calibration by the SVN using the TSC and R-TSC in the above Example <2-4-2>.

As demonstrated in Examples <3-1-4> and <3-2-4>, the calibration of data sets by the SVN using the calibration coefficient can result in the same calibration effect as those by the SVN using the TSC directly or the TSC in combination with the R-TSC.

### Example 4: Calibration of Data Set by Reference Signal-Based Normalization (RSN) and Analysis of Calibrated Data Set

In this Example, the method of Reference Signal-based Normalization (RSN) using a signal value at a reference cycle was used for calibrating variations in amplified signals of data sets. The RSN method was named for a process using a background-representing signal value of a data set as described above.

The signal variations in the following three groups of data sets were compared and analyzed: (i) a group of data sets obtained from an instrument without a hardware adjustment; (ii) a group of data sets obtained from an instrument with a hardware adjustment; and (iii) a group of data sets software-wise calibrated by the RSN or IBS-RSN.

<4-1> Preparation of Data Set

The raw data sets obtained in Example <1-1> were used.

<4-2> Measurement of Instrument Blank Signal

Raw data sets include generally both signals from a fluorescent molecule and an instrument blank signal generated in the absence of a fluorescent molecule. Accordingly, it is preferable to measure an instrument blank signal and then subtract it from raw data sets in order to utilize signals originated only from the fluorescent molecule and thus obtain more accurate results.

In this Example, the signal measured from an empty tube was used as the instrument blank signal.

The measurement of an instrument blank signal may be performed around temperature for detecting signals of a real-time PCR or may be performed with or without repetition of an amplification cycle. In this Example, 10 cycles of the amplification were performed under the same condition as described in Example <1-1> and the signal value measured at the 10^{th} cycle was used as the instrument blank signal. The instrument blank signal for each instrument was measured as shown in Table 11.

TABLE 11

| | Instrument Blank Signal of | Instrument Blank Signal of |
|---|---|---|
| Instrument | Instrument without Hardware | Instrument with Hardware |
| Number | Adjustment | Adjustment |
| Instrument 1 | RFU 2525 | RFU 2977 |
| Instrument 2 | RFU 3152 | RFU 3638 |
| Instrument 3 | RFU 3629 | RFU 3010 |

<4-3> Analysis of Data Set Obtained from Instrument without Hardware Adjustment

As in the above Example <1-2>, the intra-instrument coefficients of variations of the amplification signals of the instruments 1, 2, and 3 were analyzed as 5.2%, 9.1%, and 4.5%, respectively and the inter-instrument coefficient of variation of the amplification signals of the instruments 1, 2, and 3 was analyzed as 49.3% (FIG. 2B).

<4-4> Analysis of Data Set Obtained from Instrument with Hardware Adjustment

As in the above Example <1-3>, the intra-instrument coefficients of variations of the amplification signals of the instruments 1, 2, and 3 were analyzed as 5.3%, 7.8%, and 4.8%, respectively and the inter-instrument coefficient of variation of the amplification signals of the instruments 1, 2, and 3 was analyzed as 17.7% (FIG. 3B).

<4-5> Analysis of Data Set Software-Wise Calibrated Using RSN

The Reference Signal-based Normalization (RSN) is to calibrate a data set using a signal value at a reference cycle of the data set instead of the total signal change value (TSC) of the standard data set used in the above Examples 1 to 3. The raw data sets of six groups obtained in Example <1-1> were software-wise calibrated using the RSN according to the following steps:

<Step 1>

A specific cycle in the background region (baseline region) of the raw data set was designated as a reference cycle. In this Example, the 5^{th} cycle was designated as the reference cycle and the signal value at the reference cycle of each data set was designated as the reference signal (RS).

<Step 2>

The signal values at all cycles of each data set were calibrated using the RS of each data set.

Calibrated Signal Value (RFU)=Signal Value of Raw Data Set (RFU)÷RS

The calibrated six groups of data sets were obtained by calibrating the six groups of the raw data sets provided in Example <1-1> according to the above steps 1 to 2.

A. Analysis of the Results of Calibration of Data Sets Obtained from an Instrument without a Hardware Adjustment

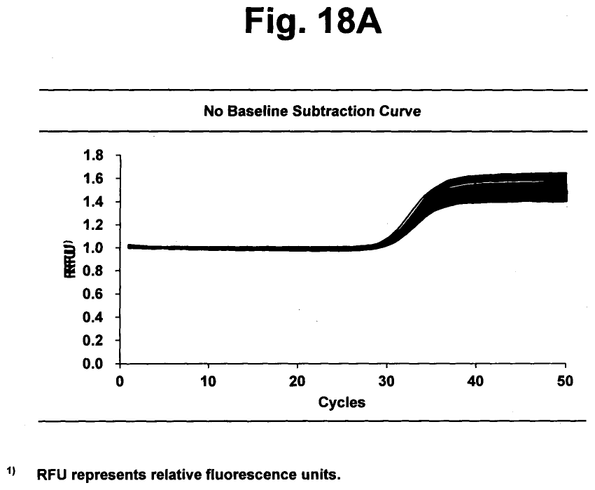

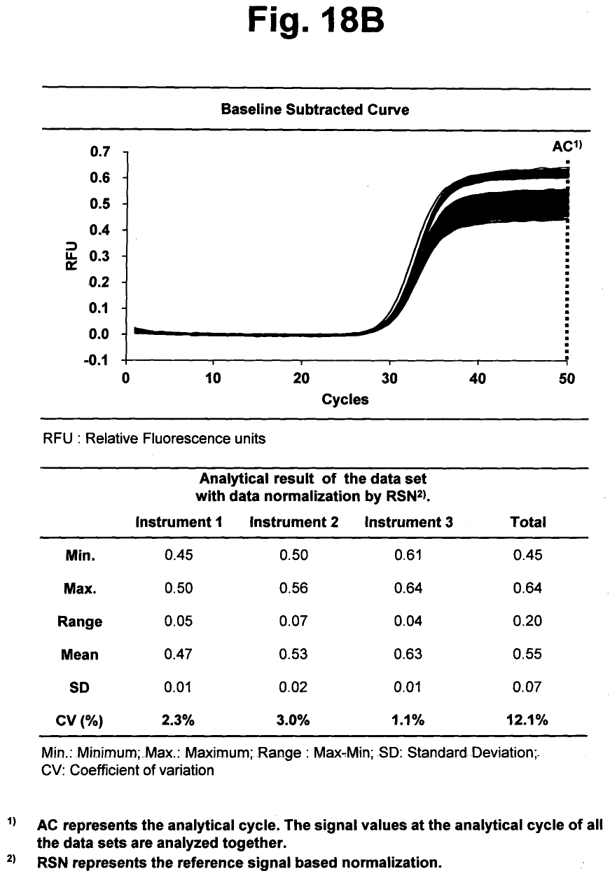

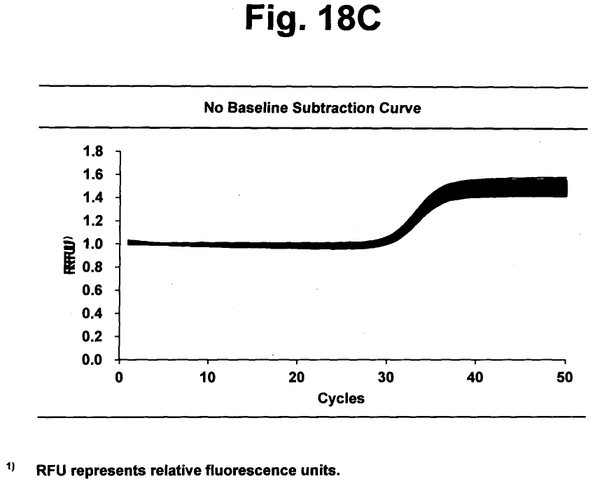

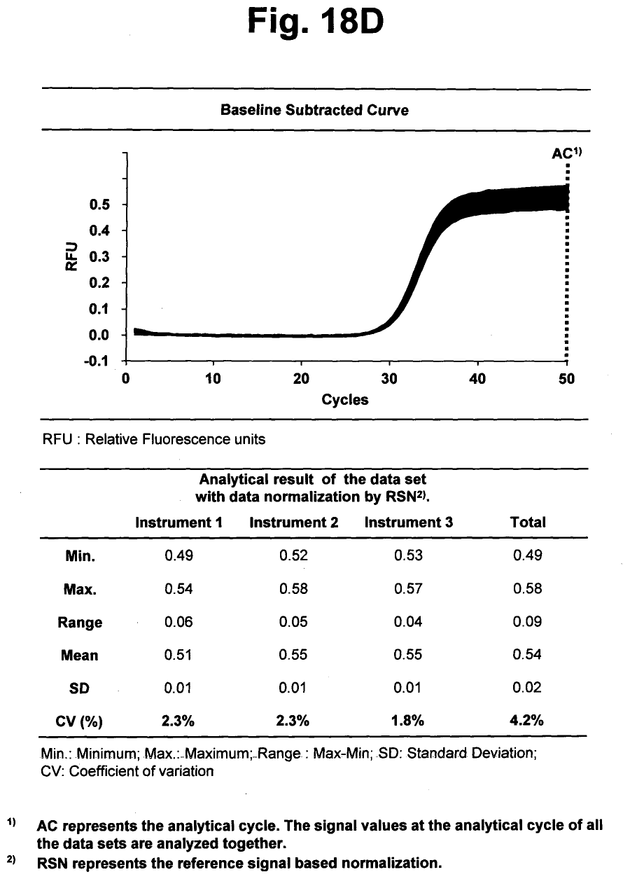

The data sets obtained from an instrument without a hardware adjustment were calibrated by the RSN using the reference signal, and the resulting calibrated data sets were analyzed (FIGS. 18A and 18B).

As shown in FIG. 18B, the intra-instrument coefficients of variations of the amplification signals were 2.3%, 3.0% and 1.1%, respectively and the inter-instrument coefficient of variation of the amplification signal was 12.1%.

The following three coefficients of variations were compared and analyzed: (i) the coefficient of variation of the signals in the data sets obtained from the instrument without a hardware adjustment in Example <1-2>; (ii) the coefficient of variation of the signals in the data sets obtained from the instrument with hardware adjustment in Example <1-3>; and (iii) the coefficient of variation of the signals in the calibrated data sets provided by calibrating the data sets with the RSN using the RS of this Example, in which the data sets had been obtained from the instrument without a hardware adjustment.

As shown in Table 12, the calibrated data sets by the RSN using the RS have following characteristics: When compared with the data sets obtained from the instrument without a hardware adjustment, (i) the intra-instrument coefficient of variation of the amplification signals was greatly reduced by more than a half; and (ii) the inter-instrument coefficient of variation of the amplification signals was remarkably reduced by 37.2% P (percentage points). In addition, when compared with the data sets obtained from the instrument with a hardware adjustment, (i) the intra-instrument coefficient of variation of the amplification signals was greatly reduced by more than a half; and (ii) the inter-instrument coefficient of variation of the amplification signals was reduced by 5.6% P (percentage points).

It would be demonstrated that the RSN method of the invention can reduce signal variations between the wells within an instrument as well as between the instruments. In particular, it would be understood that the RSN has more excellent calibration effect than methods of adjusting hardware of an instrument, addressing that a signal calibration effect being more remarkable than that of a hardware adjustment can be successfully accomplished by the RSN even without a hardware adjustment of an instrument.

TABLE 12

| | | | | |
|---|---|---|---|---|
| Calibration | RSN Using the RSs | − | − | + |
| Method | Hardware Adjustment | − | + | − |
| Result of Analysis | Instrument 1 | 5.2 | 5.3 | 2.3 |
| of Amplification | Instrument 2 | 9.1 | 7.8 | 3.0 |
| Signal | Instrument 3 | 4.5 | 4.8 | 1.1 |
| (Coefficient of | Total | 49.3 | 17.7 | 12.1 |
| Variation, CV %) | | | | |

B. Analysis of the Results of Calibration of Data Sets Obtained from an Instrument with a Hardware Adjustment

The data sets obtained from an instrument with a hardware adjustment were calibrated by the RSN using the reference signal, and the resulting calibrated data sets were analyzed (FIGS. 18C and 18D).

As shown in FIG. 18D, the intra-instrument coefficients of variations of the amplification signals were 2.3%, 2.3% and 1.8%, respectively and the inter-instrument coefficient of variation of the amplification signal was 4.2%.

The following three coefficients of variations were compared and analyzed: (i) the coefficient of variation of the signals in the data sets obtained from the instrument without a hardware adjustment in Example <1-2>; (ii) the coefficient of variation of the signals in the data sets obtained from the instrument with hardware adjustment in Example <1-3>; and (iii) the coefficient of variation of the signals in the calibrated data sets provided by calibrating the data sets with the RSN using the RS of this Example, in which the data sets had been obtained from the instrument with a hardware adjustment.

As shown in Table 13, the calibrated data sets by the RSN using the RS have following characteristics: When compared with the data sets obtained from the instrument without a hardware adjustment, (i) the intra-instrument coefficient of variation of the amplification signals was greatly reduced by more than a half; and (ii) the inter-instrument coefficient of variation of the amplification signals was remarkably reduced by 45.1% P (percentage points). In addition, when compared with the data sets obtained from the instrument with a hardware adjustment, (i) the intra-instrument coefficient of variation of the amplification signals was greatly reduced by more than a half; and (ii) the inter-instrument coefficient of variation of the amplification signals was reduced by 13.5% P (percentage points).

It would be demonstrated that the RSN method of the invention can reduce the signal variations between the wells within an instrument as well as between the instruments. Interestingly, data sets obtained from the instrument with a hardware adjustment can be further calibrated by the RSN, such that an inter- and intra-instrument variation can be more precisely corrected.

TABLE 13

| | | | | |
|---|---|---|---|---|
| Calibration | RSN Using the RSs | − | − | + |
| Method | Hardware Adjustment | − | + | + |
| Result of | Instrument 1 | 5.2 | 5.3 | 2.3 |
| Analysis | Instrument 2 | 9.1 | 7.8 | 2.3 |
| of Amplification | Instrument 3 | 4.5 | 4.8 | 1.8 |
| Signal | Total | 49.3 | 17.7 | 4.2 |
| (Coefficient | | | | |
| of Variation, CV %) | | | | |

<4-6> Analysis of Data Set Software-Wise Calibrated Using IBS-RSN

The calibration of the data sets using the RSN will be more accurate if a signal value corresponding to an instrument blank signal is subtracted from the data sets. In this Example, the method of Instrument Blank signal Subtraction and Reference Signal-based Normalization (IBS-RSN) was used for the calibration of the amplified signal variations. The raw data sets of six groups obtained in Example <1-1> were software-wise calibrated using the IBS-RSN according to the following steps:

<Step 1>

The 1^{st} calibrated data set was obtained by subtracting the instrument blank signal of Example <4-2> from the raw data sets of Example <1-1> as the following equation:

1^{st} Calibrated Data Set=Raw Data set −Instrument Blank Signal

By the calculation using the above equation, a total of six groups of 1^{st} calibrated data sets were provided, including three groups of the 1^{st} calibrated data sets obtained by using the raw data sets of the instruments without a hardware adjustment and the other three groups of the 1^{st} calibrated data sets obtained by using the raw data sets of the instruments with a hardware adjustment.

<Step 2>

A specific cycle in the background region (baseline region) of the 1^{st} Calibrated Data Sets was designated as a reference cycle. In this Example, the 5^{th} cycle or the region from 3^{rd} to 5^{th} cycles was designated as the reference cycle (RC). The signal values the 5^{th} cycle or the average signal values in the region from 3^{rd} to 5^{th} cycles of each data set was designated as the reference signal (RS) of each data set.

<Step 3>

The signal values at all cycles from each data set were calibrated using the RS obtained from each data set.

2^{nd} Calibrated Signal Value (RFU)=Signal Value of 1^{st} Calibrated Data Set (RFU)÷RS

The 2^{nd} calibrated six groups of data sets were obtained by calibrating the six groups of the 1^{st} Calibrated Data Sets provided according to the above steps 1 to 3.

A. Analysis of the Results of Calibration of Data Sets Obtained from an Instrument without a Hardware Adjustment

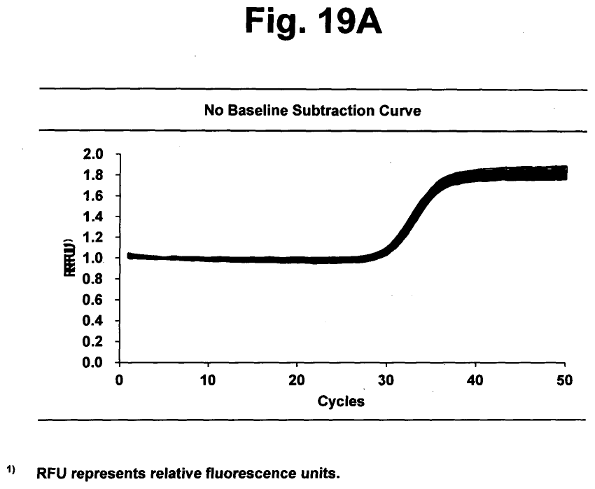

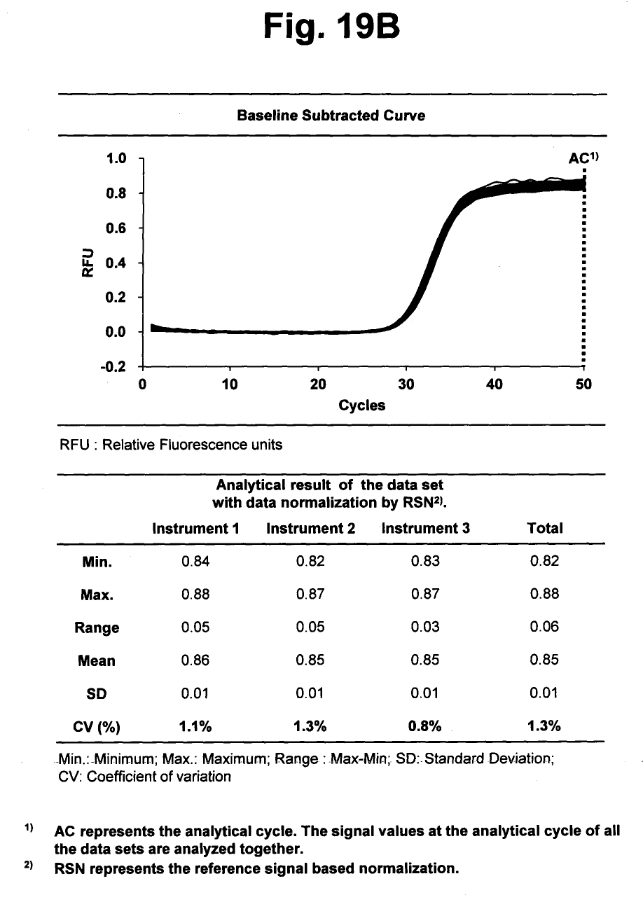

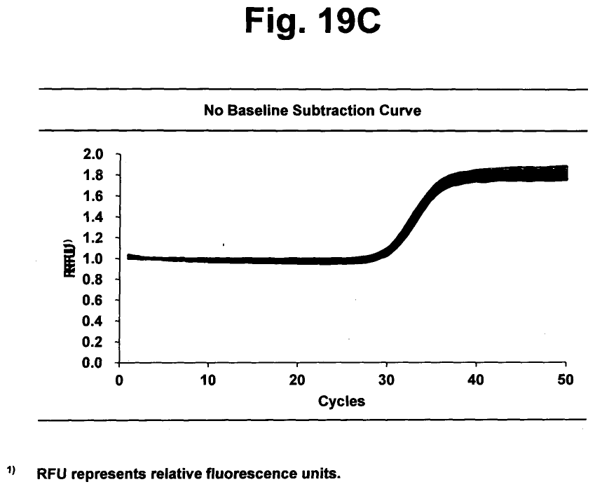

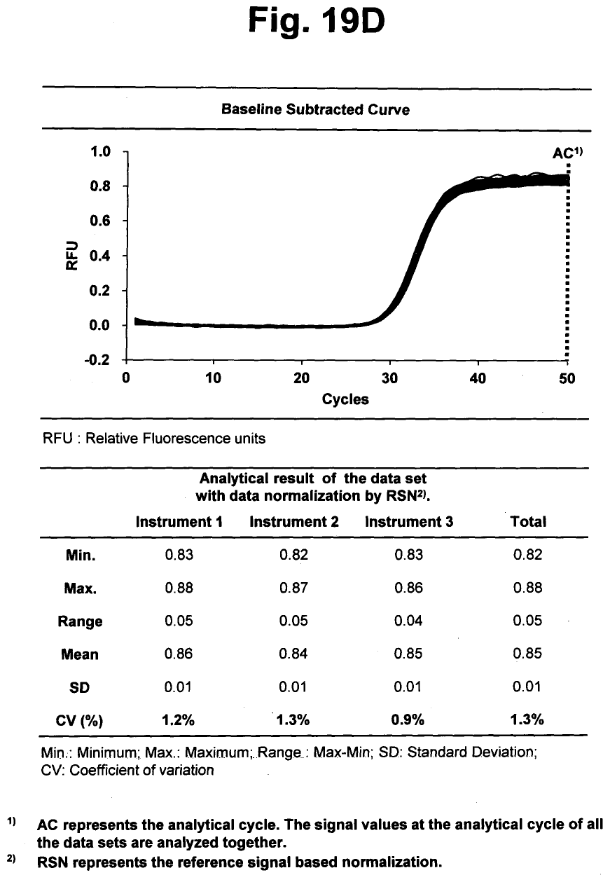

The data sets obtained from an instrument without a hardware adjustment were calibrated by the RSN using the reference signal, and the resulting calibrated data sets were analyzed (FIGS. 19A-D).

The results of analysis of the data sets calibrated by the RSN using the signal value at the 5^{th} cycle as the reference signal were shown in FIGS. 19A and 19B.

As shown in FIG. 19B, the intra-instrument coefficients of variations of the amplification signals were 1.1%, 1.3% and 0.8%, respectively and the inter-instrument coefficient of variation of the amplification signal was 1.3%.

The results of the analysis of the data sets calibrated by the RSN using the average signal value in the region from 3^{rd} to 5^{th} cycles as the reference signal were shown in FIGS. 19C and 19D.

As shown in FIG. 19D, the intra-instrument coefficients of variations of the amplification signals were 1.2%, 1.3% and 0.9%, respectively and the inter-instrument coefficient of variation of the amplification signal was 1.3%.

The following four coefficients of variations were compared and analyzed: (i) the coefficient of variation of the signals in the data sets obtained from the instrument without a hardware adjustment in Example <1-2>; (ii) the coefficient of variation of the signals in the data sets obtained from the instrument with hardware adjustment in Example <1-3>; (iii) the coefficient of variation of the signals in the calibrated data sets provided by calibrating the data sets with the RSN using the reference signal from the one reference cycle in this Example; and (iv) the coefficient of variation of the signals in the calibrated data sets provided by calibrating the data sets with the RSN using the average reference signal from the two or more reference cycles in this Example, in which the data sets had been obtained from the instrument without a hardware adjustment.

As shown in Table 14, the calibrated data sets by the RSN using the reference signal have following characteristics: When compared with the data sets obtained from the instrument without a hardware adjustment, (i) the intra-instrument coefficient of variation of the amplification signal was reduced by more than a half; and (ii) the inter-instrument coefficient of variation of the amplification signal was remarkably reduced by 48.0% P (percentage points). In addition, when compared with the data sets obtained from the instrument with a hardware adjustment, (i) the inter-instrument coefficient of variation of the amplification signals was reduced by more than a half; and (ii) the inter-instrument coefficient of variation of the amplification signals was significantly reduced by 16.4% P (percentage points).

It would be demonstrated that the signal calibration method of the invention using the IBS-RSN can reduce both the inter-instrument signal variations and the inter-well signal variations within an instrument. In particular, it would be understood that the IBS-RSN had superior calibration effects to the method of calibrating the instrument in hardware-wise, addressing that a signal calibration effect better than that of the hardware calibration can be successfully accomplished by using only the IBS-RSN without a hardware adjustment of the instrument.

TABLE 14

| | | | | | |
|---|---|---|---|---|---|
| Calibration | RSN Using the RSs | − | − | + | + |
| Method | | | | (RS at 5^{th} | (RS from 3^{rd} to |
| | | | | cycle) | 5^{th} cycles) |
| | Hardware | − | + | − | − |
| | Adjustment | | | | |
| Result of | Instrument 1 | 5.2 | 5.3 | 1.1 | 1.2 |
| Analysis | Instrument 2 | 9.1 | 7.8 | 1.3 | 1.3 |
| of Amplification | Instrument 3 | 4.5 | 4.8 | 0.8 | 0.9 |
| Signal | Total | 49.3 | 17.7 | 1.3 | 1.3 |
| (Coefficient | | | | | |
| of Variation, CV %) | | | | | |

B. Analysis of the Results of Calibration of Data Sets Obtained from an Instrument with a Hardware Adjustment

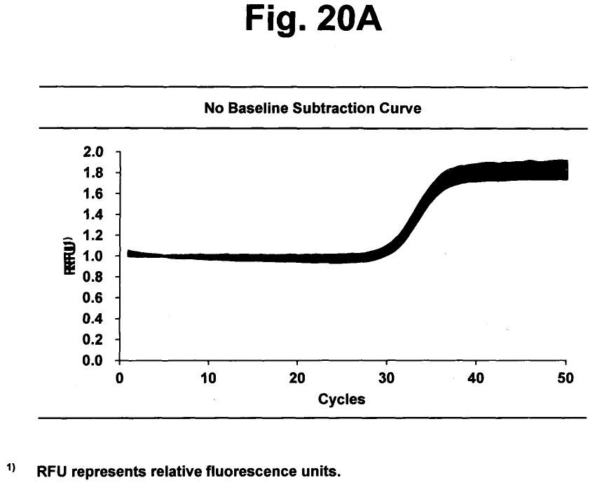

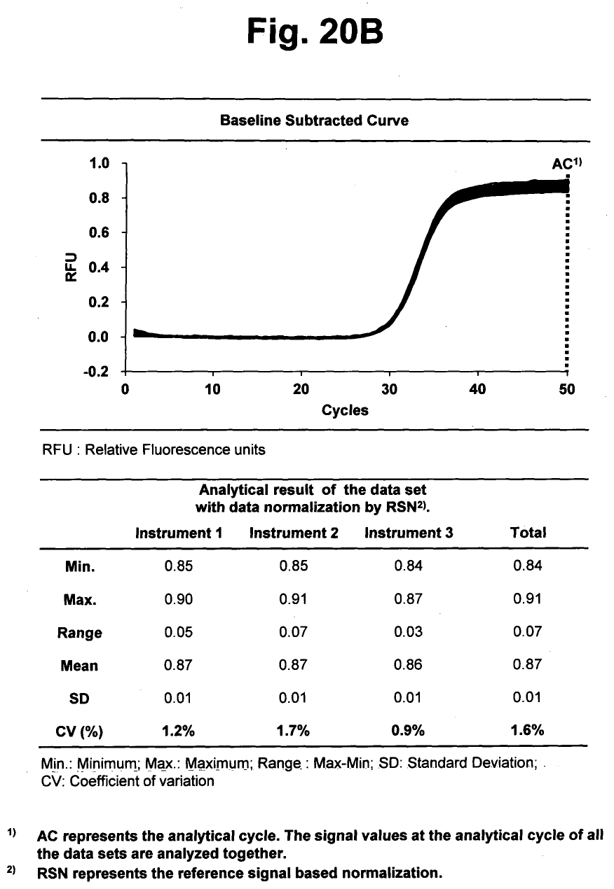

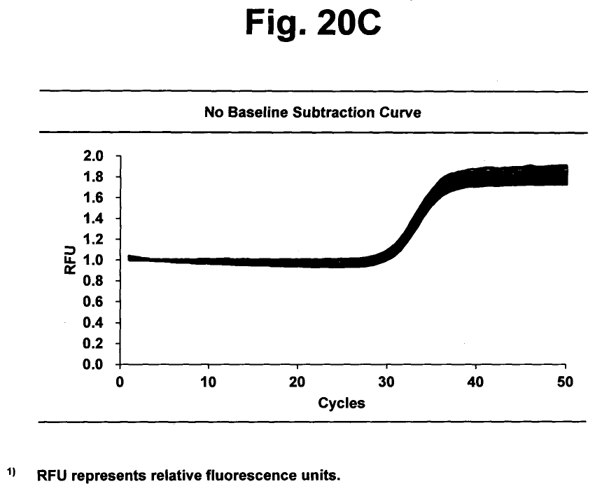

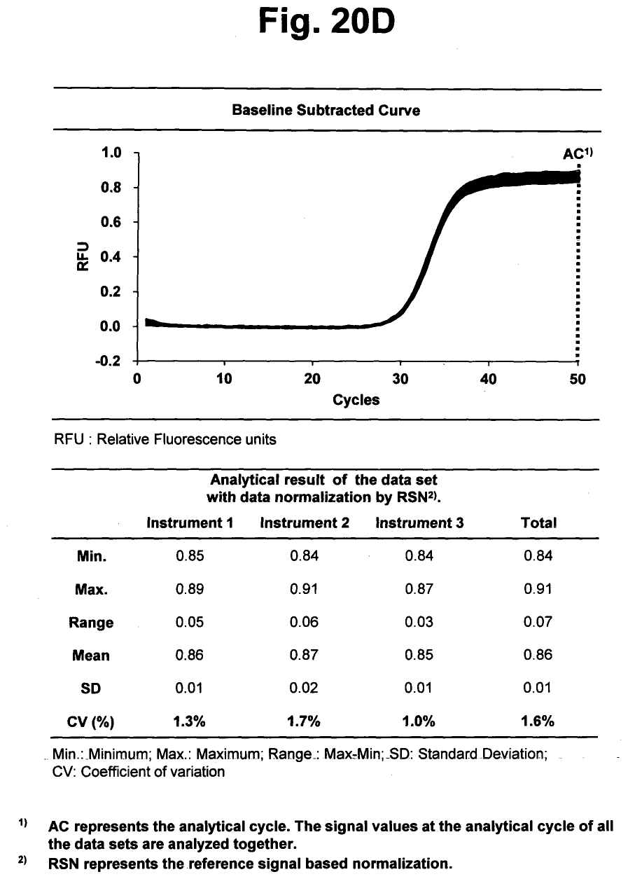

The data sets obtained from an instrument with a hardware adjustment were calibrated by the RSN using the reference signal, and the resulting calibrated data sets were analyzed (FIGS. 20A and 20D).

The results of analysis of the data sets calibrated by the RSN using the signal value at the 5^{th} cycle as the reference signal were shown in FIGS. 20A and 20B.

As shown in FIG. 20B, the intra-instrument coefficients of variations of the amplification signals were 1.2%, 1.7% and 0.9%, respectively and the inter-instrument coefficient of variation of the amplification signal was 1.6%.

The results of analysis of the data sets calibrated by the RSN using the average signal value in the region from 3^{rd} to 5^{th} cycles as the reference signal were shown in FIGS. 20C and 20D.

As shown in FIG. 20D, the intra-instrument coefficients of variations of the amplification signals were 1.3%, 1.7% and 1.0%, respectively and the inter-instrument coefficient of variation of the amplification signal was 1.6%.

The following four coefficients of variations were compared and analyzed: (i) the coefficient of variation of the signals in the data sets obtained from the instrument without a hardware adjustment in Example <1-2>; (ii) the coefficient of variation of the signals in the data sets obtained from the instrument with hardware adjustment in Example <1-3>; (iii) the coefficient of variation of the signals in the calibrated data sets provided by calibrating the data sets with the RSN using the reference signal from the one reference cycle in this Example; and (iv) the coefficient of variation of the signals in the calibrated data sets provided by calibrating the data sets with the RSN using the reference signal from the two or more reference cycles in this Example, in which the data sets had been obtained from the instrument with a hardware adjustment.

As shown in Table 15, the calibrated data sets by the RSN using the reference signal have following characteristics: When compared with the data sets obtained from the instrument without a hardware adjustment, (i) the intra-instrument coefficient of variation of the amplification signal was reduced by more than a half; and (ii) the inter-instrument coefficient of variation of the amplification signal was remarkably reduced by 47.7% P (percentage points). In addition, when compared with the data sets obtained from the instrument with a hardware adjustment, (i) the inter-instrument coefficient of variation of the amplification signals was reduced by more than a half; and (ii) the inter-instrument coefficient of variation of the amplification signals was significantly reduced by 16.1% P (percentage points).

It would be demonstrated that the signal calibration method of the invention using the IBS-RSN can effectively reduce both the inter-instrument signal variations and the inter-well signal variations within an instrument. Interestingly, data sets obtained from the instrument with a hardware adjustment can be further calibrated by the IBS-RSN, such that an inter- and intra-instrument variation can be more precisely corrected.

TABLE 15

| | | | | | |
|---|---|---|---|---|---|
| Calibration | RSN Using the RSs | − | − | + | + |
| Method | | | | (RS at 5^{th} | (RS from 3^{rd} to |
| | | | | cycle) | 5^{th} cycles) |
| | Hardware | − | + | + | + |
| | Adjustment | | | | |
| Result of | Instrument 1 | 5.2 | 5.3 | 1.2 | 1.3 |
| Analysis | Instrument 2 | 9.1 | 7.8 | 1.7 | 1.7 |
| of Amplification | Instrument 3 | 4.5 | 4.8 | 0.9 | 1.0 |
| Signal | Total | 49.3 | 17.7 | 1.6 | 1.6 |
| (Coefficient | | | | | |
| of Variation, CV %) | | | | | |

The present method of calibrating the signals of a real-time PCR instrument using the IBS-RSN can be utilized to reduce the intra- and inter-instrument signal variations with convenient and software-wise approach and has a superior calibration effect to the method using the RSN.

Having described a preferred embodiment of the present invention, it is to be understood that variants and modifications thereof falling within the spirit of the invention may become apparent to those skilled in this art, and the scope of this invention is to be determined by appended claims and their equivalents.

1. A method for calibrating a data set of a target analyte in a sample comprising:
(a) providing an analyte-insusceptible signal value for calibrating the data set; wherein the data set is obtained from a signal-generating process for the target analyte using a signal-generating means; wherein the data set comprises a plurality of data points comprising cycles and signal values of the signal-generating process; wherein the analyte-insusceptible signal value is provided (i) by a background-representing signal value of the data set; wherein the background-representing signal value is provided by a signal value at a reference cycle of the data set and the reference cycle is selected within a background region of the data set where signal generation is insusceptible to the presence or absence of the target analyte in the sample; or (ii) by a total signal change value of a standard data set; wherein the standard data set is obtained by a signal-generating process for a standard material of the target analyte; and
(b) providing a calibrated data set by obtaining calibrated signal values by applying the analyte-insusceptible signal value to the signal values of the data set.

2. The method according to claim 1, wherein the analyte-insusceptible signal value is (i) the background-representing signal value of the data set or (ii) the total signal change value of the standard data set obtained by the signal-generating process for the standard data set.

3. The method according to claim 1, wherein the calibrated data set in the step (b) is provided by obtaining calibrated signal values by dividing the signal values of the data set by the analyte-insusceptible signal value.

4. The method according to claim 1, wherein the data set of the target analyte has information indicating the presence or absence of the target analyte in the sample.

5. The method according to claim 1, wherein the signal-generating process generates signals in a dependent manner on the presence of the target analyte in the sample.

6. The method according to claim 1, wherein the signal-generating process is a process amplifying the signal value.

7. The method according to claim 1, wherein the target analyte is a target nucleic acid molecule.

8. (canceled)

9. The method according to claim 1, wherein the signal-generating process is a polymerase chain reaction (PCR) or a real-time polymerase chain reaction (real-time PCR).

10. The method according to claim 1, wherein the signal-generating process comprises a plurality of signal-generating processes for the same-typed target analyte performed in different reaction vessels; wherein the data set comprises a plurality of data sets obtained from the plurality of signal-generating processes.

11. The method according to claim 10, wherein the reference cycle is selected from a reference cycle group of each data set, wherein the reference cycle group of each data set is provided in the same manner to each other.

12. The method according to claim 10, wherein an identical reference cycle is provided for calibrating each data set of the plurality of data sets.

13. (canceled)

14. The method according to claim 10, wherein a total signal change value of a representative standard data set is provided identically for calibrating each of the plurality of the data sets.

15. The method according to claim 10, wherein the plurality of the signal-generating processes is performed on different instruments from each other.

16. The method according to claim 1, wherein the data set of the step (a) is a modified data set.

17. The method according to claim 1, wherein the data set of the step (a) is a data set of which a blank signal value is removed.

18. The method according to claim 17, wherein the blank signal value is a signal value obtained by no use of the signal-generating means.

19. (canceled)

20. The method according to claim 1, wherein the method further comprises the following step before the step (a): performing the signal-generating process to obtain a data set of the target analyte in the sample.

21. A computer readable storage medium containing instructions to configure a processor to perform a method for calibrating a data set of a target analyte in a sample, the method comprising:
(a) providing an analyte-insusceptible signal value for calibrating the data set; wherein the data set is obtained from a signal-generating process for the target analyte using a signal-generating means; wherein the data set comprises a plurality of data points comprising cycles and signal values of the signal-generating process; wherein the analyte-insusceptible signal value is provided (i) by a background-representing signal value of the data set; wherein the background-representing signal value is provided by a signal value at a reference cycle of the data set and the reference cycle is selected within a background region of the data set where signal generation is insusceptible to the presence or absence of the target analyte in the sample; or (ii) by a total signal change value of a standard data set; wherein the standard data set is obtained by a signal-generating process for a standard material of the target analyte; and
(b) providing a calibrated data set by obtaining calibrated signal values by applying the analyte-insusceptible signal value to the signal values of the data set.

22. A device for analyzing a method for calibrating a data set of a target analyte in a sample, comprising (a) a computer processor and (b) the computer readable storage medium of claim 21 coupled to the computer processor.

23. A computer program to be stored on a computer readable storage medium to configure a processor to perform a method for calibrating a data set of a target analyte in a sample, the method comprising:
(a) providing an analyte-insusceptible signal value for calibrating the data set; wherein the data set is obtained from a signal-generating process for the target analyte using a signal-generating means; wherein the data set comprises a plurality of data points comprising cycles and signal values of the signal-generating process; wherein the analyte-insusceptible signal value is provided (i) by a background-representing signal value of the data set; wherein the background-representing signal value is provided by a signal value at a reference cycle of the data set and the reference cycle is selected within a background region of the data set where signal generation is insusceptible to the presence or absence of the target analyte in the sample; or (ii) by a total signal change value of a standard data set; wherein the standard data set is obtained by a signal-generating process for a standard material of the target analyte; and
(b) providing a calibrated data set by obtaining calibrated signal values by applying the analyte-insusceptible signal value to the signal values of the data set.
